SEQUENCE LISTING (2 sequences)

SEQ ID NO: 1 - DNA, 987 bp, Homo sapiens
accaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 2 - protein, 328 aa, Homo sapiens
TKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2007/0111281 A1
(43) Pub. Date: May 17, 2007

(54) Antigen binding molecules having modified Fc regions and altered binding to Fc receptors

(75) Inventors: Peter Sondermann, Rudolfstetten-Friedlisberg (CH); Claudia Ferrara Koller, Zurich (CH); Peter Brunker, Hittnau (CH); Fiona Stuart, Zurich (CH); Pablo Umana, Zurich (CH)

Correspondence Address: STERNE, KESSLER, GOLDSTEIN & FOX P.L.L.C., 1100 NEW YORK AVENUE, N.W., WASHINGTON, DC, 20005, US

(73) Assignee: GLYCART BIOTECHNOLOGY AG, Schlieren-Zurich (CH)

(21) Appl. No.: 11/430,283

(22) Filed: May 9, 2006

Related U.S. Application Data
(60) Provisional application No. 60/678,776, filed on May 9, 2005.

(51) Int. Cl. C12P 21/06; C07H 21/04; C07K 16/28; C12N 5/06

(52) U.S. Cl. 435/69.1; 435/326; 435/320.1; 530/387.1; 536/23.53

(57) ABSTRACT

The present invention is directed to antigen binding molecules, including antibodies, comprising a Fc region having one or more amino acid modifications, wherein the antigen binding molecule exhibits altered binding to one or more Fc receptors as a result of the modification(s). The invention is further directed to polynucleotides and vectors encoding such antigen binding molecules, to host cells comprising the same, to methods for making the antigen binding molecules of the invention, and to their use in the treatment of various diseases and disorders, e.g., cancers.

### CROSS-REFERENCE TO RELATED APPLICATIONS

This application claims the benefit of U.S. Provisional Application No. 60/678,776, filed May 9, 2005, the entire contents of which are hereby incorporated by reference.

### BACKGROUND OF THE INVENTION

1. Field of the Invention

The present invention is directed to antigen binding molecules, including antibodies, comprising a Fc region having one or more amino acid modifications, wherein the antigen binding molecule exhibits altered binding to one or more Fc receptors as a result of the modification(s). The invention is further directed to polynucleotides and vectors encoding such antigen binding molecules, to host cells comprising same, to methods for making the antigen binding molecules of the invention, and to their use in the treatment of various diseases and disorders, e.g., cancers.

2. Background of the Invention

Antibodies provide a link between the humoral and the cellular immune system with IgG being the most abundant serum immunoglobulin. While the Fab regions of the antibody recognize antigens, the Fc portion binds to Fcγ receptors (FcγRs) that are differentially expressed by all immune competent cells. Upon receptor crosslinking by a multivalent antigen/antibody complex, degranulation, cytolysis or phagocytosis of the target cell and transcriptional-activation of cytokine-encoding genes are triggered (Deo, Y. M. et al., Immunol. Today 18(3):127-135 (1997)).

The effector functions mediated by the antibody Fc region can be divided into two categories: (1) effector functions that operate after the binding of antibody to an antigen (these functions involve, for example, the participation of the complement cascade or Fc receptor (FcR)-bearing cells); and (2) effector functions that operate independently of antigen binding (these functions confer, for example, persistence in the circulation and the ability to be transferred across cellular barriers by transcytosis). For example, binding of the Cl component of complement to antibodies activates the complement system. Activation of complement is important in the opsonisation and lysis of cell pathogens. The activation of complement also stimulates the inflammatory response and may also be involved in autoimmune hypersensitivity. Further, antibodies bind to cells via the Fc region, with an Fc receptor binding site on the antibody Fc region binding to a Fc receptor (FcR) on a cell. There are a number of Fc receptors which are specific for different classes of antibody, including IgG (gamma receptors), IgE (epsilon receptors), IgA (alpha receptors) and IgM (mu receptors). While the present invention is not limited to any particular mechanism, binding of antibody to Fc receptors on cell surfaces triggers a number of important and diverse biological responses including engulfment and destruction of antibody-coated particles, clearance of immune complexes, lysis of antibody-coated target cells by killer cells (known as antibody-dependent cell-mediated cytotoxicity, or ADCC), release of inflammatory mediators, placental transfer and control of immunoglobulin production.

FcRs are defined by their specificity for immunoglobulin isotypes; Fc receptors for IgG antibodies are referred to as FcγR, for IgE as FcεR, for IgA as FcαR and so on. Three subclasses of human FcγR have been identified: FcγRI (CD64), FcγRII (CD32) and FcγRIII (CD16).

Because each FcγR subclass is encoded by two or three genes, and alternative RNA splicing leads to multiple transcripts, a broad diversity in FcγR isoforms exists. The three genes encoding the FcγRI subclass (FcγRIA, FcγRIB and FcγRIC) are clustered in region 1q21.1 of the long arm of chromosome 1; the genes encoding FcγRII isoforms (FcγRIIA, FcγRIIB and FcγRIIC) and the two genes encoding FcγRIII (FcγRIIIA and FcγRIIIB) are all clustered in region 1q22. These different FcR subtypes are expressed on different cell types (see, e.g., Ravetch, J. V. and Kinet, J. P. Annu. Rev. Immunol. 9: 457-492 (1991)). For example, in humans, FcγRIIIB is found only on neutrophils, whereas FcγRIIIA is found on macrophages, monocytes, natural killer (NK) cells, and a subpopulation of T-cells. Notably, FcγRIIIA is the only FcR present on NK cells, one of the cell types implicated in ADCC.

FcγRI, FcγRII and FcγRIII are immunoglobulin superfamily (IgSF) receptors; FcγRI has three IgSF domains in its extracellular domain, while FcγRII and FcγRIII have only two IgSF domains in their extracellular domains.

Another type of Fc receptor is the neonatal Fc receptor (FcRn). FcRn is structurally similar to major histocompatibility complex (MHC) and consists of an α-chain non-covalently bound to β2-microglobulin.

Recently the importance of the activating receptor FcγRIIIa for the in vivo elimination of tumor cells was discovered. In follicular non-Hodgkin's lymphoma patients a relationship was reported between the FcγRIIIa genotype and clinical and molecular responses to rituximab, an anti-CD20 chimeric antibody used against haematological malignancies (Cartron, G. et al., Blood 99(3):754-758 (2002)). The authors demonstrated that the efficacy of rituximab was higher in patients homozygous for the “high affinity”-FcγRIIIa, characterized by a valine at position 158 (FcγRIIIa[Val-158]), than in patients heterozygous or homozygous for the “low affinity”-FcγRIIIa, which has a phenylalanine residue at this position (FcγRIIIa[Phe-158]). This dissimilarity seems to account for the significantly different affinities for the antibody displayed by FcγRIIIa-positive immune cells (Dall'Ozzo, S. et al., Cancer Res. 64(13):4664-4669 (2004)).

The above observations imply a crucial role for FcγRIIIa in the elimination of tumor cells and support the idea that monoclonal antibodies (mAbs) with increased affinity for FcγRIIIa will have improved biological activity. One route to enhance the affinity towards FcγRIIIa and consequently the effector functions of monoclonal antibodies is the manipulation of their carbohydrate moiety (Umaña, P. et al., Nat. Biotech. 17(2):176-180 (1999), Shields, R. L. et al., J. Biol. Chem. 277(30):26733-26740 (2002), Ferrara, C. et al., submitted). The N-glycosylation of Fc at Asn-297 in both Cγ2 domains is crucial to the affinity to all FcγRs (Tao, M. H. & Morrison, S. L., J. Immunol. 143(8):2595-2601 (1989), Mimura, Y., et al., J. Biol. Chem. 276(49):45539-45547 (2001) and to elicit proper effector functions (Wright, A. & Morrison, S. L., J. Exp. Med. 180(3):1087-1096 (1994), Sarmay, G. et al., Mol. Immunol. 29(5):633-639 (1992)). It is comprised of a conserved pentasaccharide structure with variable addition of fucose and outer arm sugars (Jefferis, R. et al., Immunol. Rev. 163:59-76 (1998)). The N-glycosylation pattern of mAbs has been manipulated by engineering the glycosylation pathway of a production cell line using enzyme activities that lead to naturally occurring carbohydrates. The resulting glycoengineered (GE) antibodies feature high proportions of bisected, non-fucosylated oligosaccharides, improved affinity for FcγRIIIa and enhanced ADCC (Umana, P. et al., Nat. Biotech. 17(2):176-180 (1999), Ferrara, C. et al., submitted). Similar results are found using a production cell line which is unable to add fucose residues to N-linked oligosaccharides (Sarmay, G. et al., Mol. Immunol. 29(5):633-639 (1992).

In contrast to the situation with IgG Fc, little information is available on the influence of FcγRIIIa glycosylation on receptor activity. The crystal structure of unglycosylated FcγRIII in complex with the Fc fragment of hIgG1 indicates that the putative carbohydrate moiety of FcγRIII potentially attached at Asn-162 would point into the central cavity within the Fc fragment (Shields, R. L. et al., J. Biol. Chem. 277(30):26733-26740 (2002)), where the rigid core glycans attached to IgG-Asn-297 are also located (Huber, R. et al., Nature 264(5585):415-420 (1976)). This arrangement suggests a possible approach of the carbohydrate moieties of both proteins upon complex formation.

To dissect the interaction between IgG1 and soluble human (sh) FcγRIIIa on a molecular level, binding of shFcγRIIIa variants to distinct antibody glycovariants was evaluated by surface plasmon resonance (SPR) and in a cellular system.

### SUMMARY OF THE INVENTION

In one embodiment, the invention is directed to a glycoengineered antigen-binding molecule comprising a Fc region, wherein said Fc region has an altered oligosaccharide structure as a result of said glycoengineering and has at least one amino acid modification, and wherein said antigen binding molecule exhibits increased binding to a human FcγRIII receptor compared to the antigen binding molecule that lacks said modification. In a preferred embodiment, the glycoengineered antigen binding molecule does not exhibit increased binding to a human FcγRII receptor, such as the FcγRIIa receptor or the FcγRIIb receptor.

Preferably, the FcγRIII receptor is glycosylated such that it comprises N-linked oligosaccharides at Asn162. In one embodiment, the FcγRIII receptor is FcγRIIIa. In another embodiment, the FcγRIII receptor is FcγRIIIb. In certain embodiments, the FcγRIIIa receptor has a valine residue at position 158. In other embodiments, the FcγRIIIa receptor has a phenylalanine residue at position 158.

In a preferred embodiment, the glycoengineered antigen binding molecule of the present invention contains a modification that does not substantially increase binding to a nonglycosylated FcγRIII receptor compared to the antigen binding molecule lacking said modification. In one embodiment, the glycoengineered antigen binding molecule of the present invention comprises a substitution at one or more of amino acids 239, 241, 243, 260, 262, 263, 264, 265, 268, 290, 292, 293, 294, 295, 296, 297, 298, 299, 300, 301, 302, or 303. In some embodiments, the glycoengineered antigen binding molecule comprises two or more of the substitutions listed in Tables 2 and 4. In some embodiments, the glycoengineered antigen binding molecule comprises the two or more substitutions listed in Table 5.

The present invention is further directed to a glycoengineered antigen binding molecule comprising one or more substitutions that replace the naturally occurring amino acid residue with an amino acid residue that interacts with the carbohydrate attached to Asn162 of the FcγRIII receptor. Preferably, the amino acid residue that interacts with the carbohydrate attached to Asn162 of the FcγRIII receptor is selected from the group consisting of: Trp, His, Tyr, Glu, Arg, Asp, Phe, Asn, and Gln.

In a preferred embodiment, the glycoengineered antigen binding molecule comprises a substitution selected from the group consisting of: Ser239Asp, Ser239Glu, Ser239Trp, Phe243His, Phe243Glu, Thr260His, His268Asp, His268Glu. Alternatively or additionally, the glycoengineered antigen binding molecule according to the present invention may contain one or more substitutions listed in Tables 2 or 4.

In a preferred embodiment, the glycoengineered antigen binding molecule of the present invention binds to the FcγRIII receptor with at least 10% increased affinity, at least 20% increased affinity, at least 30% increased affinity, at least 40% increased affinity, at least 50% increased affinity, at least 60% increased affinity, at least 70% increased affinity, at least 80% increased affinity, at least 90%, increased affinity, or at least 100% increased affinity compared to the same antigen binding molecule lacking said modification.

The glycoengineered antigen binding molecule of the present invention preferably comprises a human IgG Fc region. In one embodiment, the antigen binding molecule is an antibody or an antibody fragment comprising an Fc region. In a preferred embodiment, the antibody or antibody fragment is chimeric or humanized.

In certain embodiments, the glycoengineered antigen binding molecule according to the invention exhibits increased effector function. Preferably, the increased effector function is increased antibody-dependent cellular cytotoxicity or increased complement dependent cytotoxicity.

The altered oligosaccharide structure in the glycoengineered antigen binding molecules of the present invention preferably comprises a decreased number of fucose residues as compared to the nonglycoengineered antigen binding molecule. In a preferred embodiment, at least 20%, at least 30%, at least 40%, at least 50%, at least 60%, at least 70%, at least 80% or more of the oligosaccharides in the Fc region are nonfucosylated.

The altered oligosaccharide structure in the glycoengineered antigen binding molecules of the present invention may also comprise an increased number of bisected oligosaccharides as compared to the nonglycoengineered antigen binding molecule. The bisected oligosaccharide may be of the hybrid type or the complex type. The present invention also encompasses a glycoengineered antigen binding molecule, wherein said altered oligosaccharide structure comprises an increase in the ratio of GlcNAc residues to fucose residues as compared to the nonglycoengineered antigen binding molecule.

In a preferred embodiment, the glycoengineered antigen binding molecules of the present invention selectively bind an antigen selected from the group consisting of: the human CD20 antigen, the human EGFR antigen, the human MCSP antigen, the human MUC-1 antigen, the human CEA antigen, the human HER2 antigen, and the human TAG-72 antigen.

The present invention is also directed to a glycoengineered antigen binding molecule comprising a Fc region, wherein said Fc region has an altered oligosaccharide structure as a result of said glycoengineering and has at least one amino acid modification, and wherein said antigen binding molecule exhibits increased specificity to a human FcγRIII receptor compared to the antigen binding molecule that lacks said modification. Preferably, the glycoengineered antigen binding molecule of the present invention does not exhibit increased specificity to a human FcγRII receptor, such as the human FcγRIIa receptor or the human FcγRIIb receptor.

The FcγRIII receptor is preferably glycosylated, (i.e., it comprises N-linked oligosaccharides at Asn162). In one embodiment, the FcγRIII receptor is FcγRIIIa. In an alternative embodiment, the FcγRIII receptor is FcγRIIb. In certain embodiments, the FcγRIIIa receptor has a valine residue at position 158. In other embodiments, the FcγRIIIa receptor has a phenylalanine residue at position 158.

In a preferred embodiment, the amino acid modification of an antigen binding molecule does not substantially increase specificity for a nonglycosylated FcγRIII receptor compared to the antigen binding molecule lacking the modification.

In a particularly preferred embodiment, the modification comprises an amino acid substitution at one or more of amino acid positions 239, 241, 243, 260, 262, 263, 264, 265, 268, 290, 292, 293, 294, 295, 296, 297, 298, 299, 300, 301, 302, or 303. In a preferred embodiment, the substitution replaces the naturally occurring amino acid residue with an amino acid residue that interacts with the carbohydrate attached to Asn162 of the FcγRIII receptor. In one embodiment, the amino acid residue that interacts with the carbohydrate attached to Asn162 of the FcγRIII receptor is selected from the group consisting of: Trp, His, Tyr, Glu, Arg, Asp, Phe, Asn, and Gln.

In one embodiment, the substitution is selected from the group consisting of: Ser239Asp, Ser239Glu, Ser239Trp, Phe243His, Phe243Glu, Thr260His, His268Asp, His268Glu. The glycoengineered antigen binding molecule according to the present invention may also contain one or more of the substitutions listed in Tables 2 or 5.

In a preferred embodiment, the invention encompasses a glycoengineered antigen binding molecule wherein said antigen binding molecule binds to a FcγRIII receptor with at least 10% increased specificity, at least 20% increased specificity, at least 30% increased specificity, at least 40% increased specificity, at least 50% increased specificity, at least 60% increased specificity, at least 70% increased specificity, at least 80% increased specificity, at least 90% increased specificity, or at least 100% increased specificity or more compared to the antigen binding molecule lacking said modification.

Preferably, the glycoengineered antigen binding molecule of the invention exhibiting increased specificity contains a human IgG Fc region. In another preferred embodiment, the antigen binding molecule is an antibody or an antibody fragment comprising an Fc region. In a particularly preferred embodiment, the antibody or antibody fragment is chimeric or humanized.

The glycoengineered antigen binding molecule according to the invention preferably exhibits increased effector function, e.g., increased antibody-dependent cellular cytotoxicity or increased complement dependent cytotoxicity.

The altered oligosaccharide structure may comprise a decreased number of fucose residues as compared to the nonglycoengineered antigen binding molecule. For example, the invention encompasses a glycoengineered antigen binding molecule, wherein at least 20%, at least 30%, at least 40%, at least 50%, at least 60%, at least 70%, at least 80%, at least 90% or more of the oligosaccharides in the Fc region are nonfucosylated.

In another embodiment, the altered oligosaccharide structure may comprise an increased number of bisected oligosaccharides as compared to the nonglycoengineered antigen binding molecule. The bisected oligosaccharides may be of hybrid type or the complex type. In one embodiment, the altered oligosaccharide structure comprises an increase in the ratio of GlcNAc residues to fucose residues as compared to the nonglycoengineered antigen binding molecule.

In a preferred embodiment, the glycoengineered antigen binding molecules according to the invention selectively bind an antigen selected from the group consisting of: the human CD20 antigen, the human EGFR antigen, the human MCSP antigen, the human MUC-1 antigen, the human CEA antigen, the human HER2 antigen, and the human TAG-72 antigen.

The present invention is also directed to a polynucleotide encoding a polypeptide comprising an antibody Fc region or a fragment of an antibody Fc region, wherein said Fc region or fragment thereof has at least one amino acid modification, and wherein said polypeptide exhibits increased binding to a human FcγRIII receptor compared to the same polypeptide that lacks said modification. The present invention is also directed to polypeptides encoded by such polynucleotides. The polypeptide may be an antibody heavy chain. The polypeptide may also be fusion protein.

The present invention is further directed to vectors and host cells comprising the polynucleotides of the invention.

The present invention is also directed to a method for producing a glycoengineered antigen binding molecule comprising a Fc region, wherein said Fc region has an altered oligosaccharide structure as a result of said glycoengineering and has at least one amino acid modification, and wherein said antigen binding molecule exhibits increased binding to a human FcγRIII receptor compared to the antigen binding molecule that lacks said modification, said method comprising:
(i) culturing the host cell of the invention under conditions permitting the expression of said polynucleotide; and
(ii) recovering said glycoengineered antigen binding molecule from the culture medium.

The invention is also directed to a method for producing a glycoengineered antigen binding molecule comprising a Fc region, wherein said Fc region has an altered oligosaccharide structure as a result of said glycoengineering and has at least one amino acid modification, and wherein said antigen binding molecule exhibits increased selectivity to a human FcγRIII receptor compared to the antigen binding molecule that lacks said modification, said method comprising:
(i) culturing the host cell of the invention under conditions permitting the expression of said polynucleotide; and
(ii) recovering said glycoengineered antigen binding molecule from the culture medium.

### BRIEF DESCRIPTION OF THE DRAWINGS

FIG. 1(a-c). Oligosaccharide characterization of glycoengineered (GE) and native antibodies: (a) Carbohydrate moiety associated with the Asn297 of human IgG1-Fc. The sugars in bold define the pentasaccharide core; the addition of the other sugar residues is variable. The bisecting β1,4-linked GlcNAc residue is introduced by GnT-III. (b) MALDI-MS spectra of neutral oligosaccharides released from native and GE antibodies. The m/z value corresponds to the sodium-associated oligosaccharide ion. To confirm the carbohydrate type the antibodies were treated with Endoglycosidase H which only hydrolyzes hybrid but not complex glycans. (c) Oligosaccharide distributions of the IgG glycovariants used in this study. Glyco-1 refers to a glycoengineered antibody variant generated from overexpression of GnT-III alone. Glyco-2 refers to a glycoengineered antibody variant generated by co-expression of GnT-E and recombinant ManII.

FIG. 2(a-b). Binding of the shFcγRIIa[Val-158] or shFcγRIIIa[Phe-158] to immobilized IgG1 glycovariants. The association phase is represented by a solid bar above the curves. (a) Overlay of sensograms of the binding events for shFcγRIIIa[Val-158] and shFcγRIIa[Phe-158], respectively. To compare the binding event of GE antibodies within a similar response range, the sensograms obtained at concentrations of 800 nM or 6.4 μM for the native antibody were overlaid. All sensograms were normalized to the immobilization level. (b) Kinetic analysis for shFcγRIIIa[Val-158] or shFcγRIIIa[Phe-158] binding to Glyco-2. Fitted curves and residual errors (below) were derived by non-linear curve fitting.

FIG. 3(a-c). Binding of IgG glycovariants to hFcγRIIIa[Val-158/Gln-162]. All sensograms were normalized to the immobilization level. (a) Overlay of sensograms of the binding events for shFcγRIIIa[Val-158/Gln-162]. The association phase is represented by a solid bar above the curves. (b) Overlay of sensograms of the binding events for shFcγRIIIa[Val-158/Gln-162] or shFcγRIIIa[Val-158] binding to WT or Glyco-2. (c) Whole-cell binding of IgG to hFcγRIIIa[Val-158/Gln-162]- and hFcγRIIIa[Val158]-expressing or untransfected Jurkat cells. FcγRIIIa binding is given in arbitrary units.

FIG. 4(a-b). The proposed interaction of the glycosylated FcγRIII with the Fc-fragment of IgG. (a) The crystal structure of FcγRIII in complex with the Fc-fragment of native IgG (PDB code 1e4k) is shown in the inset. The rectangle indicates the clipping shown above. The two chains of the Fc fragment and the unglycosylated FcγRIII are depicted as surface with Asn162 and the fucose residue indicated. The glycans attached to the Fc are shown as ball and sticks. The fucose residue linked to the carbohydrate of the Fc fragment chain is responsible for the sterical hindrance of the proposed interaction with the FcγRIII carbohydrate. (b) Model of interaction between a glycosylated FcγRIII and the (non-fucosylated) Fc fragment of GE-IgG. As the fucose residue is not present within GE-IgG, the carbohydrates attached at Asn162 of the receptor can thoroughly interact with the GE-IgG. The figure was created using the program PYMOL (www.delanoscientific.com).

### DETAILED DESCRIPTION OF THE INVENTION

Terms are used herein as generally used in the art, unless otherwise defined as follows.

ABBREVIATIONS: Ig, Immununoglobulin; ADCC, Antibody-dependent cellular cytotoxicity; CDC, Complement-dependant cytotoxicity; PBMC, Peripheral blood mononuclear cells; GE, Glyco-engineered; GlcNAc, N-Acetylglucosamine; Man, mannose; Gal, galactose; Fuc, fucose; NeuAc, N-acetylneuraminic acid; GnT-III, N-acetylglucosaminyltransferase III; kₒₙ, association rate constant; k_{off}, dissociation rate constant.

As used herein, the term antibody is intended to include whole antibody molecules, including monoclonal, polyclonal and multispecific (e.g., bispecific) antibodies, as well as antibody fragments having the Fc region and retaining binding specificity and at least one effector function, e.g., ADCC, and fusion proteins that include a region functionally equivalent to the Fc region of an immunoglobulin and that retain binding specificity and at least one effector function. Also encompassed are chimeric and humanized antibodies, as well as camelized and primatized antibodies.

As used herein, the term Fc region is intended to refer to a C-terminal region of a human IgG heavy chain. Although the boundaries of the Fc region of an IgG heavy chain might vary slightly, the human IgG heavy chain Fc region is usually defined to stretch from the amino acid residue at position Cys226 to the carboxyl-terminus.

As used herein, the term region equivalent to the Fc region of an immunoglobulin is intended to include naturally occurring allelic variants of the Fc region of an immunoglobulin as well as variants having alterations which produce substitutions, additions, or deletions but which do not decrease substantially the ability of the immunoglobulin to mediate effector functions (such as antibody dependent cellular cytotoxicity). For example, one or more amino acids can be deleted from the N-terminus or C-terminus of the Fc region of an immunoglobulin without substantial loss of biological function. Such variants can be selected according to general rules known in the art so as to have minimal effect on activity. (See, e.g., Bowie, J. U. et al., Science 247:1306-10 (1990)).

As used herein, the term antigen binding molecule or ABM refers in its broadest sense to a molecule that specifically binds an antigenic determinant. Preferably, the ABM is an antibody; however, single chain antibodies, single chain Fv molecules, Fab fragments, diabodies, triabodies, tetrabodies, and the like are also contemplated by the present invention.

By specifically binds or binds with the same specificity when used to describe an antigen binding molecule of the invention is meant that the binding is selective for the antigen and can be discriminated from unwanted or nonspecific interactions.

As used herein, the terms fusion and chimeric, when used in reference to polypeptides such as ABMs refer to polypeptides comprising amino acid sequences derived from two or more heterologous polypeptides, such as portions of antibodies from different species. For chimeric ABMs, for example, the non-antigen binding components may be derived from a wide variety of species, including primates such as chimpanzees and humans. The constant region of the chimeric ABM is most preferably substantially identical to the constant region of a natural human antibody; the variable region of the chimeric antibody is most preferably substantially identical to that of a recombinant antibody having the amino acid sequence of the murine variable region. Humanized antibodies are a particularly preferred form of fusion or chimeric antibody.

As used herein, a polypeptide having, for example, GnT-III activity refers to a polypeptide that is able to catalyze the addition of a N-acetylglucosamine (GlcNAc) residue in β-1-4 linkage to the β-linked mannoside of the trimannosyl core of N-linked oligosaccharides. This includes fusion polypeptides exhibiting enzymatic activity similar to, but not necessarily identical to, an activity of β(1,4)-N-acetylglucosaminyltransferase III, also known as β-1,4-mannosyl-glycoprotein 4-β-N-acetylglucosaminyl-transferase (EC 2.4.1.144), according to the Nomenclature Committee of the International Union of Biochemistry and Molecular Biology (NC-IUBMB), as measured in a particular biological assay, with or without dose dependency. In the case where dose dependency does exist, it need not be identical to that of GnT-III, but rather substantially similar to the dose dependence in a given activity as compared to the GnT-III (i.e., the candidate polypeptide will exhibit greater activity or not more than about 25 fold less and, preferably, not more than about tenfold less activity, and most preferably, not more than about three fold less activity relative to the GnT-III.)

As used herein, the term variant (or analog) refers to a polypeptide differing from a specifically recited polypeptide of the invention by amino acid insertions, deletions, and substitutions, created using, e.g., recombinant DNA techniques. Variants of the ABMs of the present invention include chimeric, primatized, or humanized antigen binding molecules wherein one or several of the amino acid residues are modified by substitution, addition and/or deletion in such manner that does not substantially affect antigen binding affinity or antibody effector function. Guidance in determining which amino acid residues may be replaced, added, or deleted without abolishing activities of interest, may be found by comparing the sequence of the particular polypeptide with that of homologous peptides and minimizing the number of amino acid sequence changes made in regions of high homology (conserved regions) or by replacing amino acids with consensus sequences.

Alternatively, recombinant variants encoding these same or similar polypeptides may be synthesized or selected by making use of the “redundancy” in the genetic code. Various codon substitutions, such as the silent changes which produce various restriction sites, may be introduced to optimize cloning into a plasmid or viral vector or expression in a particular prokaryotic or eukaryotic system. Mutations in the polynucleotide sequence may be reflected in the polypeptide or domains of other peptides added to the polypeptide to modify the properties of any part of the polypeptide, to change characteristics such as ligand-binding affinities, interchain affinities, or degradation/turnover rate.

Preferably, amino acid “substitutions” are the result of replacing one amino acid with another amino acid having similar structural and/or chemical properties, i.e., conservative amino acid replacements. “Conservative” amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. “Insertions” or “deletions” are preferably in the range of about 1 to about 20 amino acids, more preferably about 1 to about 10 amino acids. The variation allowed may be experimentally determined by systematically making insertions, deletions, or substitutions of amino acids in a polypeptide molecule using recombinant DNA techniques and assaying the resulting recombinant variants for activity.

As used herein, the term humanized is used to refer to an antigen binding molecule (ABM) derived from a non-human antigen binding molecule, for example, a murine antibody, that retains or substantially retains the antigen binding properties of the parent molecule but which is less immunogenic in humans. This may be achieved by various methods including (a) grafting only the non-human complementarity determining regions (CDRs) onto human framework and constant regions with or without retention of critical framework residues (e.g., those that are important for retaining good antigen binding affinity or antibody functions), or (b) transplanting the entire non-human variable domains, but “cloaking” them with a human-like section by replacement of surface residues. Such methods are disclosed in Jones et al., Nature 321:6069, 522-525 (1986); Morrison et al., Proc. Natl. Acad. Sci. 81:6851-6855 (1984); Morrison and Oi, Adv. Immunol. 44:65-92 (1988); Verhoeyen et al., Science 239:1534-1536 (1988); Padlan, Molec. Immun. 28:489-498 (1991); Padlan, Molec. Immun. 31(3):169-217 (1994), all of which are incorporated by reference in their entirety herein.

There are generally three CDRs (CDR1, CDR2, and CDR3) in each of the heavy and light chain variable domains of an antibody, which are flanked by four framework subregions (i.e., FR1, FR2, FR3, and FR4): FR1-CDR1-FR2-CDR2-FR3-CDR3-FR4. A discussion of humanized antibodies can be found, inter alia, in U.S. Pat. No. 6,632,927, and in published U.S. application No. 2003/0175269, both of which are incorporated herein by reference in their entirety.

Similarly, as used herein, the term primatized is used to refer to an antigen binding molecule derived from a non-primate antigen binding molecule, for example, a murine antibody, that retains or substantially retains the antigen binding properties of the parent molecule but which is less immunogenic in primates.

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference, where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth below in Table 1 as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE 1
CDR DEFINITIONS¹

| | Kabat | Chothia | AbM |
|---|---|---|---|
| VH CDR1 | 31-35 | 26-32 | 26-35 |
| VH CDR2 | 50-65 | 52-58 | 50-58 |
| VH CDR3 | 95-102 | 95-102 | 95-102 |
| VL CDR1 | 24-34 | | |
| VL CDR2 | 50-56 | | |
| VL CDR3 | 89-97 | | |

¹Numbering of all CDR definitions in Table 1 is according to the numbering conventions set forth by Kabat et al. (see below).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth in Kabat et al., U.S. Dept. of Health and Human Services, “Sequence of Proteins of Immunological Interest” (1983) (incorporated herein by reference in its entirety). The sequences of any sequence listing (i.e., SEQ ID NO:1 to SEQ ID NO:2) are not numbered according to the Kabat numbering system. However, as stated above, it is well within the ordinary skill of one in the art to determine the Kabat numbering scheme of any variable region sequence in the Sequence Listing based on the numbering of the sequences as presented therein.

By a nucleic acid or polynucleotide having a nucleotide sequence at least, for example, 95% identical, or having 95% identity, to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence.

As a practical matter, whether any particular nucleic acid molecule or polypeptide is at least 80%, 85%, 90%, 95%, 96%, 97%, 98% or 99% identical to a nucleotide sequence or polypeptide sequence of the present invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al., Comp. App. Biosci. 6:237-245 (1990). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k tuple=4, Mismatch Penalty=1, Joining Penalty=30, Randomization Group Length=0, Cutoff Score=1, Gap Penalty=5, Gap Size Penalty=0.05, Window Size=500 or the length of the subject nucleotide sequence, whichever is shorter.

If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ end of the subject sequence which are not matched/aligned with the query. In this case the percent identity calculated by FASTDB is not manually corrected. Once again, only bases on the 5′ and 3′ end of the subject sequence which are not matched/aligned with the query sequence are manually corrected for. No other manual corrections are to made for the purposes of the present invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 95%, 96%, 97%, 98% or 99% identical to a reference polypeptide can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al., Comp. App. Biosci. 6:237-245 (1990). In a sequence alignment the query and subject; sequences are either both nucleotide sequences or both amino acid sequences. The result of said global sequence alignment is in percent identity. Preferred parameters used in a FASTDB amino acid alignment are: Matrix=PAM 0, k tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are to be made for the purposes of the present invention.

As used herein, a nucleic acid that hybridizes under stringent conditions to a nucleic acid sequence of the invention, refers to a polynucleotide that hybridizes in an overnight incubation at 42° C. in a solution comprising 50% formamide, 5×SSC (750 mM NaCl, 75 mM sodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65° C.

As used herein, the term Golgi localization domain refers to the amino acid sequence of a Golgi resident polypeptide which is responsible for anchoring the polypeptide in location within the Golgi complex. Generally, localization domains comprise amino terminal “tails” of an enzyme.

As used herein, the term effector function refers to those biological activities attributable to the Fc region (a native sequence Fc region or amino acid sequence variant Fc region) of an antibody. Examples of antibody effector functions include, but are not limited to, Fc receptor binding affinity, antibody-dependent cellular cytotoxicity (ADCC), antibody-dependent cellular phagocytosis (ADCP), cytokine secretion, immune-complex-mediated antigen uptake by antigen-presenting cells, down-regulation of cell surface receptors, etc.

As used herein, the terms engineer, engineered, engineering, glycoengineer, glycoengineered, glycoengineering, and glycosylation engineering are considered to include any manipulation of the glycosylation pattern of a naturally occurring or recombinant polypeptide, such as an antigen binding molecule (ABM), or fragment thereof. Glycosylation engineering includes metabolic engineering of the glycosylation machinery of a cell, including genetic manipulations of the oligosaccharide synthesis pathways to achieve altered glycosylation of glycoproteins expressed in cells. Furthermore, glycosylation engineering includes the effects of mutations and cell environment on glycosylation. In one embodiment, the glycosylation engineering is an alteration in glycosyltransferase activity. In a particular embodiment, the engineering results in altered glucosaminyltransferase activity and/or fucosyltransferase activity.

As used herein, the term host cell covers any kind of cellular system which can be engineered to generate the polypeptides and antigen binding molecules of the present invention. In one embodiment, the host cell is engineered to allow the production of an antigen binding molecule with modified glycoforms. In a preferred embodiment, the antigen binding molecule is an antibody, antibody fragment, or fusion protein. In certain embodiments, the host cells have been further manipulated to express increased levels of one or more polypeptides having GnT-III activity. In other embodiments, the host cells have been engineered to have eliminated, reduced or inhibited core α1,6-fucosyltransferase activity. The term core α1,6-fucosyltransferase activity encompasses both expression of the core α1,6-fucosyltransferase gene as well as interaction of the core α1,6-fucosyltransferase enzyme with its substrate. Host cells include cultured cells, e.g., mammalian cultured cells, such as CHO cells, BHK cells, NS0 cells, SP2/0 cells, Y0 myeloma cells, P3X63 mouse myeloma cells, PER cells, PER.C6 cells or hybridoma cells, yeast cells, insect cells, and plant cells, to name only a few, but also cells comprised within a transgenic animal, transgenic plant, or cultured plant or animal tissue.

As used herein the term native sequence Fc region refers to an amino acid sequence that is identical to the amino acid sequence of an Fc region commonly found in nature. Exemplary native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof. Other sequences are contemplated and are readily obtained from various web sites (e.g., NCBI's web site).

The terms Fc receptor and FcR are used to describe a receptor that binds to an Fc region (e.g. the Fc region of an antibody or antibody fragment) of the functional equivalent of an Fc region. Portions of Fc receptors are specifically contemplated in some embodiments of the present invention. In preferred embodiments, the FcR is a native sequence human FcR. In other preferred embodiments, the FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIa (an “activating receptor”) and FcγRIIb (an “inhibiting receptor”), which have similar amino acid sequences that differ primarily in the cytoplasmic domains thereof. Activating receptor FcγRIIa contains an immunoreceptor tyrosine based activation motif (ITAM) in its cytoplasmic domain. Inhibiting receptor FcγRIIb contains an immunoreceptor tyrosine-based inhibition motif (ITIM) in its cytoplasmic domain. The term also includes the neonatal receptor, FcRn, which is responsible for the transfer of maternal IgGs to the fetus. An example of one Fc receptor encompassed by the present invention is the low affinity immunoglobulin gamma Fc region receptor III-A precursor (IgG Fc receptor III-2) (Fc-gamma RIII-alpha) (Fc-gamma RIIIa) (FcRIIIa) (Fc-gamma RIII) (FcRIII) (Antigen CD16-A) (FcR-10). [gi:119876], the sequence of which is set forth below:

RTEDLPKAVV FLEPQWYRVL EKDSVTLKCQ GAYSPEDNST
QWFHNESLIS SQASSYFIDA ATVDDSGEYR CQTNLSTLSD
PVQLEVHIGW LLLQAPRWVF KEEDPIHLRC HSWKNTALHK
VTYLQNGKGR KYFHHNSDFY IPKATLKDSG SYFCRGLFGS
KNVSSETVNI TITQGLAVST ISSFFPPGYQ VSFCLVMVLL
FAVDTGLYFS VKTNIRSSTR DWKDHKFKWR KDPQDK

As used herein, a polypeptide variant with altered FcR binding affinity or effector function(s) is one which has either enhanced (i.e. increased) or diminished (i.e. reduced) FcR binding activity and/or effector function compared to a parent polypeptide or to a polypeptide comprising a native sequence Fc region. A polypeptide variant which exhibits increased binding to an FcR binds at least one FcR with better affinity than the parent polypeptide. A polypeptide variant which exhibits decreased binding to an FcR, binds at least one FcR with worse affinity than a parent polypeptide. Such variants which display decreased binding to an FcR may possess little or no appreciable binding to an FcR, e.g., 0-20% binding to the FcR compared to a parent polypeptide. A polypeptide variant which binds an FcR with increased affinity compared to a parent polypeptide, is one which binds any one or more of the above identified FcRs with higher binding affinity than the parent antibody, when the amounts of polypeptide variant and parent polypeptide in a binding assay are essentially the same, and all other conditions are identical. For example, a polypeptide variant with improved FcR binding affinity may display from about 1.10 fold to about 100 fold (more typically from about 1.2 fold to about 50 fold) improvement (i.e. increase) in FcR binding affinity compared to the parent polypeptide, where FcR binding affinity is determined, for example, in an FACS-based assay or a SPR analysis (Biacore).

As used herein, an amino acid modification refers to a change in the amino acid sequence of a given amino acid sequence. Exemplary modifications include, but are not limited to, an amino acid substitution, insertion, and/or deletion. In preferred embodiments, the amino acid modification is a substitution (e.g. in an Fc region of a parent polypeptide). An amino acid modification at a specified position (e.g. in the Fc region) refers to the substitution or deletion of the specified residue, or the insertion of at least one amino acid residue adjacent the specified residue. The insertion may be N-terminal or C-terminal to the specified residue.

The term binding affinity refers to the equilibrium dissociation constant (expressed in units of concentration) associated with each Fc receptor-Fc binding interaction. The binding affinity is directly related to the ratio of the kinetic off-rate (generally reported in units of inverse time, e.g. seconds.sup.−1) divided by the kinetic on-rate (generally reported in units of concentration per unit time, e.g. molar/second). In general it is not possible to unequivocally state whether changes in equilibrium dissociation constants are due to differences in on-rates, off-rates or both unless each of these parameters are experimentally determined (e.g., by BIACORE (see www.biacore.com) or SAPIDYNE measurements)

As used herein, the term Fc-mediated cellular cytotoxicity includes antibody-dependent cellular cytotoxicity and cellular cytotoxicity mediated by a soluble Fc-fusion protein containing a human Fc-region. It is an immune mechanism leading to the lysis of “antibody-targeted cells” by “human immune effector cells”, wherein:

The human immune effector cells are a population of leukocytes that display Fc receptors on their surface through which they bind to the Fc-region of antibodies or of Fc-fusion proteins and perform effector functions. Such a population may include, but is not limited to, peripheral blood mononuclear cells (PBMC) and/or natural killer (NK) cells.

The antibody-targeted cells are cells bound by the ABMs (e.g., antibodies or Fc-fusion proteins) of the invention. In general, the antibodies or Fc fusion-proteins bind to target cells via the protein part N-terminal to the Fc region.

As used herein, the term increased Fc-mediated cellular cytotoxicity is defined as either an increase in the number of “antibody-targeted cells” that are lysed in a given time and at a given concentration of antibody or Fc-fusion protein in the medium surrounding the target cells by the mechanism of Fc-mediated cellular cytotoxicity defined above, and/or a reduction in the concentration of antibody or Fc-fusion protein in the medium surrounding the target cells required to achieve the lysis of a given number of “antibody-targeted cells” in a given time by the mechanism of Fc-mediated cellular cytotoxicity. The increase in Fc-mediated cellular cytotoxicity is relative to the cellular cytotoxicity mediated by the same antibody or Fc-fusion protein produced by the same type of host cells, using the same standard production, purification, formulation, and storage methods which are known to those skilled in the art but which have not been produced by host cells glycoengineered to express the glycosyltransferase GnT-III by the methods described herein.

By antibody having increased antibody dependent cellular cytotoxicity (ADCC) is meant an antibody, as that term is defined herein, having increased ADCC as determined by any suitable method known to those of ordinary skill in the art. One accepted in vitro ADCC assay is as follows:

1) the assay uses target cells that are known to express the target antigen recognized by the antigen binding region of the antibody;

2) the assay uses human peripheral blood mononuclear cells (PBMCs), isolated from blood of a randomly chosen healthy donor, as effector cells;

3) the assay is carried out according to following protocol:

i) the PBMCs are isolated using standard density centrifugation procedures and are suspended at 5×10⁶ cells/ml in RPMI cell culture medium;

ii) the target cells are grown by standard tissue culture methods, harvested from the exponential growth phase with a viability higher than 90%, washed in RPMI cell culture medium, labeled with 100 micro-Curies of ⁵¹Cr, washed twice with cell culture medium, and resuspended in cell culture medium at a density of 10⁵ cells/ml;

iii) 100 μl of the final target cell suspension above are transferred to each well of a 96-well microtiter plate;

iv) the antibody is serially-diluted from 4000 ng/ml to 0.04 ng/ml in cell culture medium and 50 μl of the resulting antibody solutions are added to the target cells in the 96-well microtiter plate, testing in triplicate various antibody concentrations covering the whole concentration range above;

v) for the maximum release (MR) controls, 3 additional wells in the plate containing the labeled target cells, receive 50 μl of a 2% (V/V) aqueous solution of non-ionic detergent (Nonidet, Sigma, St. Louis), instead of the antibody solution (point iv above);

vi) for the spontaneous release (SR) controls, 3 additional wells in the plate containing the labeled target cells, receive 50 μl of RPMI cell culture medium instead of the antibody solution (point iv above);

vii) the 96-well microtiter plate is then centrifuged at 50×g for 1 minute and incubated for 1 hour at 4° C.;

viii) 50 μl of the PBMC suspension (point i above) are added to each well to yield an effector:target cell ratio of 25:1 and the plates are placed in an incubator under 5% CO₂ atmosphere at 37° C. for 4 hours;

ix) the cell-free supernatant from each well is harvested and the experimentally released radioactivity (ER) is quantified using a gamma counter;

x) the percentage of specific lysis is calculated for each antibody concentration according to the formula (ER−MR)/(MR−SR)×100, where ER is the average radioactivity quantified (see point ix above) for that antibody concentration, MR is the average radioactivity quantified (see point ix above) for the MR controls (see point v above), and SR is the average radioactivity quantified (see point ix above) for the SR controls (see point vi above);

4) “increased ADCC” is defined as either an increase in the maximum percentage of specific lysis observed within the antibody concentration range tested above, and/or a reduction in the concentration of antibody required to achieve one half of the maximum percentage of specific lysis observed within the antibody concentration range tested above. The increase in ADCC is relative to the ADCC, measured with the above assay, mediated by the same antibody, produced by the same type of host cells, using the same standard production, purification, formulation, and storage methods which are known to those skilled in the art but that has not been produced by host cells engineered to overexpress GnT-III.

Variant Fc Regions

The present invention provides polypeptides, including antigen binding molecules, having modified Fc regions, nucleic acid sequences (e.g., vectors) encoding such polypeptides, methods for generating polypeptides having modified Fc regions, and methods for using same in the treatment of various diseases and disorders. Preferably, the modified Fc regions of the present invention differ from the nonmodified parent Fc region by at least one amino acid modification. The “parent”, “starting” or “nonmodified” polypeptide preferably comprises at least a portion of an antibody Fc region, and may be prepared using techniques available in the art for generating polypeptides comprising an Fc region or portion thereof. In preferred embodiments, the parent polypeptide is an antibody. The parent polypeptide may, however, be any other polypeptide comprising at least a portion of an Fc region (e.g. an antigen binding molecule). In certain embodiments, a modified Fc region may be generated (e.g. according to the methods disclosed herein) and can be fused to a heterologous polypeptide of choice, such as an antibody variable domain or binding domain of a receptor or ligand. In preferred embodiments, the polypeptides of the invention comprise an entire antibody comprising light and heavy chains having a modified Fc region.

In preferred embodiments, the parent polypeptide comprises an Fc region or functional portion thereof. Generally the Fc region of the parent polypeptide will comprise a native sequence Fc region, and preferably a human native sequence Fc region. However, the Fc region of the parent polypeptide may have one or more pre-existing amino acid sequence alterations or modifications from a native sequence Fc region. For example, the Clq binding activity of the Fc region may have been previously altered or the FcγR binding affinity of the Fc region may have been altered. In further embodiments, the parent polypeptide Fc region is conceptual (e.g. mental thought or a visual representation on a computer or on paper), and while it does not physically exist, the antibody engineer may decide upon a desired modified Fc region amino acid sequence and generate a polypeptide comprising that sequence or a DNA encoding the desired modified Fc region amino acid sequence. However, in preferred embodiments, a nucleic acid encoding an Fc region of a parent polypeptide is available (e.g. commercially) and this nucleic acid sequence is altered to generate a variant nucleic acid sequence encoding the modified Fc region.

Polynucleotides encoding a polypeptide comprising a modified Fc region may be prepared by methods known in the art using the guidance of the present specification for particular sequences. These methods include, but are not limited to, preparation by site-directed (or oligonucleotide-mediated-) mutagenesis, PCR mutagenesis, and cassette mutagenesis of an earlier prepared nucleic acid encoding the polypeptide. Site-directed mutagenesis is a preferred method for preparing substitution variants. This technique is well known in the art (see, e.g., Carter et al. Nucleic Acids Res. 13: 4431-4443 (1985) and Kunkel et. al., Proc. Natl. Acad. Sci. USA 82: 488 (1987), both of which are hereby incorporated by reference). Briefly, in carrying out site directed mutagenesis of DNA, the starting DNA is altered by first hybridizing an oligonucleotide encoding the desired mutation to a single strand of such starting DNA. After hybridization, a DNA polymerase is used to synthesize an entire second strand, using the hybridized oligonucleotide as a primer, and using the single strand of the starting DNA as a template. Thus, the oligonucleotide encoding the desired mutation is incorporated in the resulting double-stranded DNA.

PCR mutagenesis is also suitable for making amino acid sequence variants of the nonmodified starting polypeptide (see, e.g., Vallette et. al., Nuc. Acids Res. 17: 723-733 (1989), hereby incorporated by reference). Briefly, when small amounts of template DNA are used as starting material in a PCR, primers that differ slightly in sequence from the corresponding region in a template DNA can be used to generate relatively large quantities of a specific DNA fragment that differs from the template sequence only at the positions where the primers differ from the template.

Another method for preparing variants, cassette mutagenesis, is based on the technique described by Wells et al., Gene 34: 315-323 (1985), hereby incorporated by reference. The starting material is the plasmid (or other vector) comprising the starting polypeptide DNA to be modified. The codon(s) in the starting DNA to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in the starting polypeptide DNA. The plasmid DNA is cut at these sites to linearize it. A double-stranded oligonucleotide encoding the sequence of the DNA between the restriction sites but containing the desired mutation(s) is synthesized using standard procedures, wherein the two strands of the oligonucleotide are synthesized separately and then hybridized together using standard techniques. This double-stranded oligonucleotide is referred to as the cassette. This cassette is designed to have 5′ and 3′ ends that are compatible with the ends of the linearized plasmid, such that it can be directly ligated to the plasmid. This plasmid now contains the mutated DNA sequence.

Alternatively, or additionally, the desired amino acid sequence encoding a polypeptide variant can be determined, and a nucleic acid sequence encoding such amino acid sequence variant can be generated synthetically.

The amino acid sequence of the parent polypeptide may be modified in order to generate a variant Fc region with altered Fc receptor binding affinity or activity in vitro and/or in vivo and/or one or more altered effector functions, such as antibody-dependent cell-mediated cytotoxicity (ADCC) activity, in vitro and/or in vivo. The amino acid sequence of the parent polypeptide may also be modified in order to generate a modified Fc region with altered complement binding properties and/or circulation half-life.

Substantial modifications in the biological properties of the Fc region may be accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into classes based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions will entail exchanging a member of one of these classes for a member of another class. Conservative substitutions will entail exchanging a member of one of these classes for another member of the same class.

One can engineer an Fc region to produce a variant with altered binding affinity for one or more FcRs. One may, for example, modify one or more amino acid residues of the Fc region in order to alter (e.g. increase or decrease) binding to an FcR. In preferred embodiments, the modification comprises one or more of the Fc region residues identified herein (See, e.g, Table 2). Generally, one will make an amino acid substitution at one or more of the Fc region residues identified herein as effecting FcR binding in order to generate such an Fc region variant. In preferred embodiments, no more than one to about ten Fc region residues will be deleted or substituted. The Fc regions herein comprising one or more amino acid modifications (e.g. substitutions) will preferably retain at least about 80%, and preferably at least about 90%, and most preferably at least about 95% of the parent Fc region sequence or of a native sequence human Fc region.

One may also make amino acid insertion modified Fc regions, which variants have altered effector function. For example, one may introduce at least one amino acid residue (e.g. one to two amino acid residues and generally no more than ten residues) adjacent to one or more of the Fc region positions identified herein as impacting FcR binding. By adjacent is meant within one to two amino acid residues of a Fc region residue identified herein. Such Fc region variants may display enhanced or diminished FcR binding and/or effector function. In order to generate such insertion variants, one may evaluate a co-crystal structure of a polypeptide comprising a binding region of an FcR (e.g. the extracellular domain of the FcR of interest) and the Fc region into which the amino acid residue(s) are to be inserted (see, e.g., Sondermann et al. Nature 406:267 (2000); Deisenhofer, Biochemistry 20 (9): 2361-2370 (1981); and Burmeister et al., Nature 3442: 379-383, (1994), all of which are herein incorporated by reference) in order to rationally design a modified Fc region that exhibits, e.g., improved FcR binding ability.

By introducing the appropriate amino acid sequence modifications in a parent Fc region, one can generate a variant Fc region which (a) mediates one or more effector functions in the presence of human effector cells more or less effectively and/or (b) binds an Fc γ receptor (FcγR) or Fc neonatal receptor (FcRn) with better affinity than the parent polypeptide. Such modified Fc regions will generally comprise at least one amino acid modification in the Fc region.

In preferred embodiments, the parent polypeptide Fc region is a human Fc region, e.g. a native human IgG1 (A and non-A allotypes), IgG2, IgG3, or IgG4 Fc region, including all allotypes known or discovered. Such regions have sequences such as those shown in SEQ ID NOS: 1-2.

In certain embodiments, the parent polypeptide Fc region is a non-human Fc region. Non-human Fc regions include Fc regions derived from non-human species such as, but not limited to, equine, porcine, bovine, murine, canine, feline, non-human primate, and avian subjects, e.g a native non-human IgG Fc region, including all subclasses and allotypes known or discovered.

In certain embodiments, in order to generate a modified Fc region with improved effector function (e.g., ADCC), the parent polypeptide preferably has pre-existing ADCC activity (e.g., the parent polypeptide comprises a human IgG1 or human IgG3 Fc region). In some embodiments, a modified Fc region with improved ADCC mediates ADCC substantially more effectively than an antibody with a native sequence IgG1 or IgG3 Fc region.

In preferred embodiments, one or more amino acid modification(s) are introduced into the CH2 domain of the parent Fc region in order to generate a modified IgG Fc region with altered Fc γ receptor (FcγR) binding affinity or activity.

In certain embodiments, the one or more amino acid modification(s) introduced into the CH2 domain of the parent Fc region occur at those positions indicated in Table 2.

TABLE 2

| Position | Substitution |
|---|---|
| Ser239 | Ser239Trp, Ser239His, Ser239Glu, |
| | Ser239Ile, Ser239Arg, Ser239Asp, |
| | Ser239Gln, Ser239Asn, Ser239Met, |
| | Ser239Val, Ser239Leu, Ser239Phe, |
| | Ser239Tyr, Ser239Ala, Ser239Lys, |
| | Ser239Pro, Ser239Cys, Ser239Thr, |
| | Ser239Gly |
| Phe241 | Phe241Trp, Phe241His, Phe241Glu, |
| | Phe241Ile, Phe241Arg, Phe241Asp, |
| | Phe241Gln, Phe241Asn, Phe241Met, |
| | Phe241Val, Phe241Leu, Phe241Tyr, |
| | Phe241Ala, Phe241Lys, Phe241Pro, |
| | Phe241Cys, Phe241Thr, Phe241Gly, |
| | Phe241Ser |
| Phe243 | Phe243Trp, Phe243His, Phe243Glu, |
| | Phe243Ile, Phe243Arg, Phe243Asp, |
| | Phe243Gln, Phe243Asn, Phe243Met, |
| | Phe243Val, Phe243Leu, Phe243Tyr, |
| | Phe243Ala, Phe243Lys, Phe243Pro, |
| | Phe243Cys, Phe243Thr, Phe243Gly, |
| | Phe243Ser |
| Thr260 | Thr260Trp, Thr260His, Thr260Glu, |
| | Thr260Ile, Thr260Arg, Thr260Asp, |
| | Thr260Gln, Thr260Asn, Thr260Met, |
| | Thr260Val, Thr260Leu, Thr260Phe, |
| | Thr260Tyr, Thr260Ala, Thr260Lys, |
| | Thr260Pro, Thr260Cys, Thr260Gly, |
| | Thr260Ser |
| Val262 | Val262Trp, Val262His, Val262Glu, |
| | Val262Ile, Val262Arg, Val262Asp, |
| | Val262Gln, Val262Asn, Val262Met, |
| | Val262Leu, Val262Phe, Val262Tyr, |
| | Val262Ala, Val262Lys, Val262Pro, |
| | Val262Gly, Val262Ser, Val262Thr, |
| | Val262Cys |
| Val263 | Val263Trp, Val263His, Val263Glu, |
| | Val263Ile, Val263Arg, Val263Asp, |
| | Val263Gln, Val263Asn, Val263Met, |
| | Val263Leu, Val263Phe, Val263Tyr, |
| | Val263Ala, Val263Lys, Val263Pro, |
| | Val263Gly, Val263Ser, Val263Thr, |
| | Val263Cys |
| Val264 | Val264Trp, Val264His, Val264Glu, |
| | Val264Ile, Val264Arg, Val264Asp, |
| | Val264Gln, Val264Asn, Val264Met, |
| | Val264Leu, Val264Phe, Val264Tyr, |
| | Val264Ala, Val264Lys, Val264Pro, |
| | Val264Gly, Val264Ser, Val264Thr, |
| | Val264Cys |
| Asp265 | Asp265Trp, Asp265His, Asp265Glu, |
| | Asp265Ile, Asp265Arg, Asp265Gln, |
| | Asp265Asn, Asp265Met, Asp265Val, |
| | Asp265Leu, Asp265Phe, Asp265Tyr, |
| | Asp265Ala, Asp265Lys, Asp265Pro, |
| | Asp265Gly, Asp265Ser, Asp265Thr, |
| | Asp265Cys |
| His268 | His268Trp, His268Glu, His268Ile, |
| | His268Arg, His268Asp, His268Gln, |
| | His268Asn, His268Met, His268Val, |
| | His268Leu, His268Phe, His268Tyr, |
| | His268Ala, His268Lys, His268Pro, |
| | His268Gly, His268Ser, His268Thr, |
| | His268Cys |
| Lys290 | Lys290Trp, Lys290Glu, Lys290Ile, |
| | Lys290Arg, Lys290Asp, Lys290Gln, |
| | Lys290Asn, Lys290Met, Lys290Val, |
| | Lys290Leu, Lys290Phe, Lys290Tyr, |
| | Lys290Ala, Lys290His, Lys290Pro, |
| | Lys290Gly, Lys290Ser, Lys290Thr, |
| | Lys290Cys |
| Arg292 | Arg292Trp, Arg292His, Arg292Glu, |
| | Arg292Ile, Arg292Asp, Arg292Gln, |
| | Arg292Asn, Arg292Met, Arg292Val, |
| | Arg292Leu, Arg292Phe, Arg292Tyr, |
| | Arg292Ala, Arg292His, Arg292Pro, |
| | Arg292Gly, Arg292Ser, Arg292Thr, |
| | Arg292Cys |
| Glu293 | Glu293Trp, Glu293His, Glu293Ile, |
| | Glu293Arg, Glu293Asp, Glu293Gln, |
| | Glu293Asn, Glu293Met, Glu293Val, |
| | Glu293Leu, Glu293Phe, Glu293Tyr, |
| | Glu293Ala, Glu293His, Glu293Pro, |
| | Glu293Gly, Glu293Ser, Glu293Thr, |
| | Glu293Cys |
| Glu294 | Glu294Trp, Glu294His, Glu294Ile, |
| | Glu294Arg, Glu294Asp, Glu294Gln, |
| | Glu294Asn, Glu294Met, Glu294Val, |
| | Glu294Leu, Glu294Phe, Glu294Tyr, |
| | Glu294Ala, Glu294His, Glu294Pro, |
| | Glu294Gly, Glu294Ser, Glu294Thr, |
| | Glu294Cys |
| Gln295 | Gln295Trp, Gln295His, Gln295Glu, |
| | Gln295Ile, Gln295Arg, Gln295Asp, |
| | Gln295Asn, Gln295Met, Gln295Val, |
| | Gln295Leu, Gln295Phe, Gln295Tyr, |
| | Gln295Ala, Gln295His, Gln295Pro, |
| | Gln295Gly, Gln295Ser, Gln295Thr, |
| | Gln295Cys |
| Tyr296 | Tyr296Trp, Tyr296His, Tyr296Glu, |
| | Tyr296Ile, Tyr296Arg, Tyr296Asp, |
| | Tyr296Gln, Tyr296Asn, Tyr296Met, |
| | Tyr296Val, Tyr296Leu, Tyr296Phe, |
| | Tyr296Ala, Tyr296His, Tyr296Pro, |
| | Tyr296Gly, Tyr296Ser, Tyr296Thr, |
| | Tyr296Cys |
| Asn297 | Asn297Trp, Asn297His, Asn297Glu, |
| | Asn297Ile, Asn297Arg, Asn297Asp, |
| | Asn297Gln, Asn297Met, Asn297Val, |
| | Asn297Leu, Asn297Phe, Asn297Tyr, |
| | Asn297Ala, Asn297His, Asn297Pro, |
| | Asn297Gly, Asn297Ser, Asn297Thr, |
| | Asn297Cys |
| Ser298 | Ser298Trp, Ser298His, Ser298Glu, |
| | Ser298Ile, Ser298Arg, Ser298Asp, |
| | Ser298Gln, Ser298Asn, Ser298Met, |
| | Ser298Val, Ser298Leu, Ser298Phe, |
| | Ser298Tyr, Ser298Ala, Ser298His, |
| | Ser298Pro, Ser298Gly, Ser298Thr, |
| | Ser298Cys |
| Thr299 | Thr299Trp, Thr299His, Thr299Glu, |
| | Thr299Ile, Thr299Arg, Thr299Asp, |
| | Thr299Gln, Thr299Asn, Thr299Met, |
| | Thr299Val, Thr299Leu, Thr299Phe, |
| | Thr299Tyr, Thr299Ala, Thr299His, |
| | Thr299Pro, Thr299Gly, Thr299Ser, |
| | Thr299Cys |
| Tyr300 | Tyr300Trp, Tyr300His, Tyr300Glu, |
| | Tyr300Ile, Tyr300Arg, Tyr300Asp, |
| | Tyr300Gln, Tyr300Asn, Tyr300Met, |
| | Tyr300Val, Tyr300Leu, Tyr300Phe, |
| | Tyr300Ala, Tyr300His, Tyr300Pro, |
| | Tyr300Gly, Tyr300Ser, Tyr300Thr, |
| | Tyr300Cys |
| Arg301 | Arg301Trp, Arg301His, Arg301Glu, |
| | Arg301Ile, Arg301Asp, Arg301Gln, |
| | Arg301Asn, Arg301Met, Arg301Val, |
| | Arg301Leu, Arg301Phe, Arg301Tyr, |
| | Arg301Ala, Arg301His, Arg301Pro, |
| | Arg301Gly, Arg301Ser, Arg301Thr, |
| | Arg301Cys |
| Val302 | Val302Trp, Val302His, Val302Glu, |
| | Val302Ile, Val302Arg, Val302Asp, |
| | Val302Gln, Val302Asn, Val302Met, |
| | Val302Leu, Val302Phe, Val302Tyr, |
| | Val302Ala, Val302His, Val302Pro, |
| | Val302Gly, Val302Ser, Val302Thr, |
| | Val302Cys |
| Val303 | Val303Trp, Val303His, Val303Glu, |
| | Val303Ile, Val303Arg, Val303Asp, |
| | Val303Gln, Val303Asn, Val303Met, |
| | Val303Leu, Val303Phe, Val303Tyr, |
| | Val303Ala, Val303His, Val303Pro, |
| | Val303Gly, Val303Ser, Val303Thr, |
| | Val303Cys |

In certain embodiments, the one or more amino acid modification(s) introduced into the CH2 domain of the parent Fc region comprises replacing the existing residue with a residue selected from the group consisting of: Trp, His, Tyr, Glu, Arg, Asp, Phe, Asn, and Gln.

In certain embodiments, more than one amino acid modification is introduced into the CH2 domain of the parent Fc region in order to generate a modified IgG Fc region with altered FcγR binding affinity or activity by combining any of the individual modifications as listed in Table 2, such that a modification at one position can be combined with one or more additional modifications located at different positions to produce two or more modifications of the parent Fc region.

In preferred embodiments, no more than one to about ten Fc region residues will be modified. The Fc regions herein comprising one or more amino acid modifications (e.g. substitutions) will preferably retain at least about 80%, and preferably at least about 90%, and most preferably at least about 95% of the parent Fc region sequence or of a native sequence human Fc region.

In certain embodiments, one or more amino acid modification(s) introduced into the CH2 domain of the parent Fc region results in significantly reduced binding of the modified Fc region to FcγRIIIa, e.g. those modifications listed in Table 3.

TABLE 3

| Position | Substitution |
|---|---|
| Ser239 | Ser239Arg |
| Phe241 | Phe241Arg |
| Phe243 | Phe243Arg |
| Val263 | Val263Trp, Val263His, Val263Glu, |
| | Val263Arg, Val263Asp, Val263Tyr |
| Val264 | Val264Trp, Val264His, Val264Glu, |
| | Val264Arg, Val264Asp |
| Asp265 | Asp265Trp, Asp265His, Asp265Glu, |
| | Asp265Arg, Asp265Tyr |
| Glu294 | Glu294Asp |
| Gln295 | Gln295Trp, Gln295Tyr, Gln295Arg |
| Tyr296 | Tyr296Arg, Tyr296Ser |
| Ser298 | Ser298Trp, Ser298His, Ser298Glu, |
| | Ser298Arg, Ser298Asp |
| Arg 301 | Arg 301His, Arg301Glu, Arg301Asp |

In a preferred embodiment, the one or more amino acid modification(s) introduced into the CH2 domain of the parent Fc region results in a modified IgG Fc region with only slightly reduced, unaltered, or increased affinity for FcγRIIIa, e.g. those modifications listed in Table 4.

TABLE 4

| Position | Substitution |
|---|---|
| Ser239 | Ser239Asp, Ser239Glu, Ser239Trp |
| Phe243 | Phe243His, Phe243Glu |
| Thr260 | Thr260His |
| His268 | His268Asp, His268Glu |

In certain embodiments, more than one amino acid modification is introduced into the CH2 domain of the parent Fc region in order to generate a modified IgG Fc region with altered FcγR binding affinity or activity by combining any of the individual modifications as listed in Table 4, such that a modification at one position can be combined with one or more additional modifications located at different positions to produce any of the two or more, three or more, or four modifications of the parent Fc region listed in Table 5.

TABLE 5

| Position | Substitution |
|---|---|
| Ser239/Phe243 | Ser239Asp/Phe243His, |
| | Ser239Glu/Phe243His, |
| | Ser239Trp/Phe243His, |
| | Ser239Asp/Phe243Glu, |
| | Ser239Glu/Phe243Glu, |
| | Ser239Trp/Phe243Glu |
| Ser239/Thr260 | Ser239Asp/Thr260His, |
| | Ser239Glu/Thr260His, |
| | Ser239Trp/Thr260His |
| Ser239/His268 | Ser239Asp/His268Asp, |
| | Ser239Glu/His268Asp, |
| | Ser239Trp/His268Asp, |
| | Ser239Asp/His268Glu, |
| | Ser239Glu/His268Glu, |
| | Ser239Trp/His268Glu |
| Phe243/Thr260 | Phe243His/Thr260His, |
| | Phe243Glu/Thr260His |
| Phe243/His268 | Phe243His/His268Asp, |
| | Phe243Glu/His268Asp, |
| | Phe243His/His268Glu, |
| | Phe243Glu/His268Glu |
| Thr260/His268 | Thr260His/His268Asp, |
| | Thr260His/His268Glu |
| Ser239/Phe243/Thr260 | Ser239Asp/Phe243His/Thr260His |
| | Ser239Glu/Phe243His/Thr260His, |
| | Ser239Trp/Phe243His/Thr260His, |
| | Ser239Asp/Phe243Glu/Thr260His, |
| | Ser239Glu/Phe243Glu/Thr260His, |
| | Ser239Trp/Phe243Glu/Thr260His |
| Ser239/Phe243/His268 | Ser239Asp/Phe243His/His268Asp, |
| | Ser239Glu/Phe243His/His268Asp, |
| | Ser239Trp/Phe243His/His268Asp, |
| | Ser239Asp/Phe243Glu/His268Asp, |
| | Ser239Glu/Phe243Glu/His268Asp, |
| | Ser239Trp/Phe243Glu/His268Asp, |
| | Ser239Asp/Phe243His/His268Glu, |
| | Ser239Glu/Phe243His/His268Glu, |
| | Ser239Trp/Phe243His/His268Glu, |
| | Ser239Asp/Phe243Glu/His268Glu, |
| | Ser239Glu/Phe243Glu/His268Glu, |
| | Ser239Trp/Phe243Glu/His268Glu |
| Ser239/Thr260/His268 | Ser239Asp/Thr260His/His268Asp, |
| | Ser239Glu/Thr260His/His268Asp, |
| | Ser239Trp/Thr260His/His268Asp, |
| | Ser239Asp/Thr260His/His268Glu, |
| | Ser239Glu/Thr260His/His268Glu, |
| | Ser239Trp/Thr260His/His268Glu |
| Phe 243/Thr260/ | Phe243His/Thr260His/His268Asp, |
| His268 | Phe243Glu/Thr260His/His268Asp, |
| | Phe243His/Thr260His/His268Glu, |
| | Phe243Glu/Thr260His/His268Glu |
| Ser239/Phe243/ | Ser239Asp/Phe243His/Thr260His/His268Asp |
| Thr260/His268 | Ser239Glu/Phe243His/Thr260His/His268Asp, |
| | Ser239Trp/Phe243His/Thr260His/His268Asp, |
| | Ser239Asp/Phe243Glu/Thr260His/His268Asp, |
| | Ser239Glu/Phe243Glu/Thr260His/His268Asp, |
| | Ser239Trp/Phe243Glu/Thr260His/His268Asp, |
| | Ser239Asp/Phe243His/Thr260His/His268Glu, |
| | Ser239Glu/Phe243His/Thr260His/His268Glu, |
| | Ser239Trp/Phe243His/Thr260His/His268Glu, |
| | Ser239Asp/Phe243Glu/Thr260His/His268Glu, |
| | Ser239Glu/Phe243Glu/Thr260His/His268Glu, |
| | Ser239Trp/Phe243Glu/Thr260His/His268Glu |

In a preferred embodiment, the more than one amino acid modification introduced into the CH2 domain of the parent Fc region involves any combination with Thr260His as listed in Table 5.

The polypeptides of the invention having modified Fc regions may be subjected to one or more further modifications, depending on the desired or intended use of the polypeptide. Such modifications may involve, for example, further alteration of the amino acid sequence (substitution, insertion and/or deletion of amino acid residues), fusion to heterologous polypeptide(s) and/or covalent modifications. Such further modifications may be made prior to, simultaneously with, or following, the amino acid modification(s) disclosed above which result in an alteration of Fc receptor binding and/or effector function.

Alternatively or additionally, it may be useful to combine amino acid modifications with one or more further amino acid modifications that alter Clq binding and/or complement dependent cytoxicity function of the Fc region. The starting polypeptide of particular interest in this regard is one that binds to Clq and displays complement dependent cytotoxicity (CDC). Amino acid substitutions described herein may serve to alter the ability of the starting polypeptide to bind to Clq and/or modify its complement dependent cytotoxicity function (e.g. to reduce and preferably abolish these effector functions). However, polypeptides comprising substitutions at one or more of the described positions with improved Clq binding and/or complement dependent cytotoxicity (CDC) function are contemplated herein. For example, the starting polypeptide may be unable to bind Clq and/or mediate CDC and may be modified according to the teachings herein such that it acquires these further effector functions. Moreover, polypeptides with pre-existing Clq binding activity, optionally further having the ability to mediate CDC may be modified such that one or both of these activities are enhanced. Amino acid modifications that alter Clq and/or modify its complement dependent cytotoxicity function are described, for example, in WO00/42072, which is hereby incorporated by reference.

As disclosed above, one can design an Fc region or portion thereof with altered effector function, e.g., by modifying Clq binding and/or FcR binding and thereby changing CDC activity and/or ADCC activity. For example, one can generate a modified Fc region with improved Clq binding and improved FcγRIII binding (e.g. having both improved ADCC activity and improved CDC activity). Alternatively, where one desires that effector function be reduced or ablated, one may engineer a modified Fc region with reduced CDC activity and/or reduced ADCC activity. In other embodiments, one may increase only one of these activities, and optionally also reduce the other activity, e.g. to generate a modified Fc region with improved ADCC activity but reduced CDC activity and vice versa.

Another type of amino acid substitution serves to alter the glycosylation pattern of the polypeptide. This may be achieved, for example, by deleting one or more carbohydrate moieties found in the polypeptide, and/or adding one or more glycosylation sites that are not present in the polypeptide. Glycosylation of polypeptides is typically either N-linked or O-linked. N-linked refers to the attachment of the carbohydrate moiety to the side chain of an asparagine residue. The peptide sequences asparagine-X-serine and asparagine-X-threonine, where X is any amino acid except proline, are the recognition sequences for enzymatic attachment of the carbohydrate moiety to the asparagine side chain. Thus, the presence of either of these peptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

In some embodiments, the present invention provides compositions comprising a modification of a parent polypeptide having an Fc region, wherein the modified Fc region comprises at least one surface residue amino acid modification (See, e.g., Deisenhofer, Biochemistry 20(9):2361-70 (1981), and WO00/42072, both of which are hereby incorporated by reference). In other embodiments, the present invention provides compositions comprising a modification of a parent polypeptide having an Fc region, wherein the modified Fc region comprises at least one non-surface residue amino acid modification. In further embodiments, the present invention comprises a variant of a parent polypeptide having an Fc region, wherein the variant comprises at least one surface amino acid modification and at least one non-surface amino acid modification.

Assays for Polypeptides Having Modified Fc Regions

The present invention further provides various assays for screening polypeptides of the present invention having modified Fc regions. Screening assays may be used to find or confirm useful modified Fc regions. For example, polypeptides with modified Fc regions may be screened to find variants with increased FcR binding, or effector function(s) such as ADCC, or CDC activity (e.g. increased or decreased ADCC or CDC activity). Also, modified polypeptides with amino acid modifications in non-surface residues may also be screened (e.g. a modified Fc region with a least one surface amino acid modification and one non-surface amino acid modification may be screened). Also, as described below, the assays of the present invention may be employed to find or confirm modified Fc regions that have beneficial therapeutic activity in a subject (e.g. such as a human with symptoms of an antibody or immunoadhesin responsive disease). A variant of assay types may be employed to evaluate any change in a polypeptide having a modified Fc region compared to the parent polypeptide (See, screening assays provided in WO00/42072, herein incorporated by reference). Further exemplary assays are described below.

In preferred embodiments, the polypeptides having modified Fc regions of the present invention are antigen binding molecules that essentially retain the ability to bind antigen (via an unmodified antigen binding region or modified antigen binding region) compared to the nonvariant (parent) polypeptide (e.g. the binding capability is preferably no worse than about 20 fold or no worse than about 5 fold of that of the nonvariant polypeptide). The binding capability of the polypeptide variant to antigen may be determined using techniques such as fluorescence activated cell sorting (FACS) analysis or radioimmunoprecipitation (RIA), for example. For more detailed information about the binding event, a biological interaction analysis may be performed using SPR.

Fc receptor (FcR) binding assays may be employed to evaluate the polypeptides with modified Fc regions of the present invention. For example, binding of Fc receptors such as FcγRI, FcγRIIa, FcγRIIb, FcγRIII, FcRn, etc., can be measured by titrating modified polypeptide and measuring bound modified polypeptide variant using an antibody which specifically binds to the polypeptide variant in a standard ELISA format. For example, an antigen binding molecule comprising a modified Fc region of the present invention may be screened in a standard ELISA assay to determine binding to an FcR. A solid surface may be coated with an antigen. Excess antigen may be washed, and the surface blocked. The modified polypeptide (antibody) is specific for this antigen, and therefore binds to the antigen-coated surface. Then an FcR conjugated to a label (e.g. biotin) may be added, and the surface washed. In the following step a molecule specific for the label on the FcR is added (e.g. avidin conjugated to an enzyme). Thereafter a substrate may be added in order to determine the amount of binding of the FcR to the polypeptide with the modified Fc region. The results of this assay can be compared to the ability of the parent polypeptide that lacks the modification to bind the same FcR. In preferred embodiments, the FcR is selected from FcγRIIA, FcγRIIB, and FcγRIIIA for IgG, as these receptors (e.g. expressed recombinantly) may be successfully employed to screen the modified Fc regions of the present invention. In fact, such binding assays with these preferred receptors unexpectedly allows the identification of useful modified Fc regions. It is unexpected that useful modified polypeptides (e.g. with greater FcR binding or effector function(s) such as ADCC or CDC) are identified in such a fashion. In other preferred embodiments, the components for carrying out an ELISA (e.g. with FcγRIIA, FcγRIIB, and FcγRIIIA for IgG) to screen variants are packaged in a kit (e.g. with instructions for use).

Useful effector cells for such assays include, but are not limited to, natural killer (NK) cells, macrophages, and other peripheral blood mononuclear cells (PBMC). Alternatively, or additionally, ADCC activity of the polypeptides having modified Fc regions of the present invention may be assessed in vivo, e.g., in a animal model such as that disclosed in Clynes et al. PNAS (USA) 95:652-656 (1998), herein incorporated by reference).

The ability of modified polypeptides to bind Clq and mediate complement dependent cytotoxicity (CDC) may be assessed. For example, to determine Clq binding, a Clq binding ELISA may be performed. An exemplary Clq binding assay is a follows. Assay plates may be coated overnight at 4° C. with modified polypeptide of the invention or parental polypeptide (control) in coating buffer. The plates may then be washed and blocked. Following washing, an aliquot of human Clq may be added to each well and incubated for 2 hrs at room temperature. Following a further wash, 100 μl of a sheep anti-complement Clq peroxidase-conjugated antibody may be added to each well and incubated for 1 hour at room temperature. The plate may again be washed with wash buffer and 100 μl of substrate buffer containing OPD (O-phenylenediamine dihydrochloride (Sigma)) may be added to each well. The oxidation reaction, observed by the appearance of a yellow color, may be allowed to proceed for an optimized time (2-60 minutes) and stopped by the addition of 100 μl of 4.5 N H₂SO₄. The absorbance may then be read at 492 nm and the background absorbance at 405 nm subtracted from this value.

The modified Fc regions of the present invention may also be screened for complement activation. To assess complement activation, a complement dependent cytotoxicity (CDC) assay may be performed (See, e.g. Gazzano-Santoro et al., J. Immunol. Methods, 202:163 (1996), herein incorporated by reference). For example, various concentrations of the modified polypeptide of the invention and human complement may be diluted with buffer. Cells which express the antigen to which the polypeptide variant binds may be diluted to a density of ˜1×10⁶ cells/ml. Mixtures of polypeptide variant, diluted human complement and cells expressing the antigen may be added to a flat bottom tissue culture 96 well plate and allowed to incubate for 2 hours at 37° C. and 5% CO₂ to facilitate complement mediated cell lysis. 50 μl of alamar blue (Accumed International) may then be added to each well and incubated overnight at 37° C. The absorbance may be measured using a 96-well fluorimeter with excitation at 530 nm and emission at 590 nm. The results may be expressed in relative fluorescence units (RFU). The sample concentrations may be computed from a standard curve and the percent activity as compared to nonvariant polypeptide may be reported for the polypeptide variant of interest.

In preferred embodiments, the modified polypeptide has a higher binding affinity for human Clq than the parent polypeptide. Such a variant may display, for example, about two-fold or more, and preferably about five-fold or more improvement in human Clq binding compared to the parent polypeptide (e.g. at the IC50 values for these two molecules). For example, human Clq binding may be about two-fold to about 500-fold, and preferably from about two-fold or from about five-fold to about 1000-fold improved compared to the parent polypeptide.

In other preferred embodiments, variants are found that exhibit 2-fold, 25-fold, 50-fold, 100-fold or 1000-fold reduction in Clq binding compared to a control (parental) antibody having a nonmodified IgG1 Fc region. In even more preferred embodiments, the modified Fc region polypeptide does not bind Clq (e.g., 10 μg/ml of the modified polypeptide displays about 100 fold or more reduction in Clq binding compared to 10 μg/ml of the control antibody).

In certain embodiments, the modified polypeptides of the present invention do no activate complement. For example, a modified polypeptide displays about 0-10% CDC activity in this assay compared to a control antibody having a nonmodified IgG1 Fc region. Preferably the variant does not appear to have any CDC activity (e.g. above background) in the above CDC assay. In other embodiments, the modified polypeptides of the present invention are found to have enhanced CDC compared to a parent polypeptide [e.g., displaying about two-fold to about 100-fold (or greater) improvement in CDC activity in vitro or in vivo when the IC50 values are compared].

The polypeptides having modified Fc regions of the present invention may also be screened in vivo. Any type of in vivo assay may be employed. A particular example of one type of assay is provided below. This exemplary assay allows for preclinical evaluation of modified Fc regions in vivo. A modified polypeptide to be tested may be incorporated into the Fc region of a particular antibody known to have some activity. For example, a modification may be incorporated into the Fc region of an anti-CD20 IgG by mutagenesis. This allows a parental IgG and Fc variant IgG to be compared directly with RITUXAN (known to promote tumor regression). The preclinical evaluation may be done in 2 phases (a pharmacokinetic and pharmacodynamic phase). The goal of the Phase I pharmacokinetic studies is to determine if there are differences in the clearance rate between an Fc variant IgG and the antibody with known in vivo activity (e.g. RITUXAN). Differences in clearance rate may cause differences in the steady-state level of IgG in serum. As such, if differences in steady-state concentrations are detected these should be normalized to enable accurate comparisons to be made. The goal of the Phase II pharmacodynamic studies is to determine the effect of the Fc mutations upon, in this case, tumor growth. Previous studies with RITUXAN used a single dose which completely inhibited tumor growth. Because this does not allow quantitative differences to be measured, a dose range should be employed.

Phase I pharmacokinetic comparison of a polypeptide having a modified Fc region of the present invention, the nonmodified (e.g., wild type) parental Fc, and RITUXAN may be performed in the following manner. First, 40 μg per animal may be injected intravenously and the plasma level of the IgG quantitated at 0, 0.25, 0.5, 1, 24, 48, 168, and 336 hrs. The data may be fitted, for example, using a pharmacokinetic program (WinNonLin) using a zero lag two compartment pharmacokinetic model to obtain the clearance rate. Clearance rate may be used to define steady state plasma level with the following equation: C=Dose/(Clearance rate×T), where T is the interval between doses and C is the plasma level at steady state. Pharmacokinetic experiments may be performed in non-tumor bearing mice with, for example, a minimum of 5 mice per time point.

An animal model may be employed for the next phase in the following manner. The right flank of CB 17-SCID mice may be implanted with 106 Raji cells subcutaneously. Intravenous bolus of the polypeptide with modified Fc region, the polypeptide with the parent (e.g., wild type) Fc, and RITUXAN may be commenced immediately after implantation and continued until the tumor size is greater than 2 cm in diameter. Tumor volume may be determined every Monday, Wednesday and Friday by measuring the length, width, and depth of the tumor using a caliper (tumor volume=W×L×D). A plot of tumor volume versus time will give the tumor growth rate for the pharmakodynamic calculation. A minimum of about 10 animals per group should be used.

Phase II pharmacodynamic comparison of the polypeptide with modified Fc region of the invention, the parental (e.g., wild type) Fc, and RITUXAN may be performed in the following manner. Based on published data, RITUXAN at 101g/g weekly completely inhibited tumor growth in vivo (Clynes et al., Nat. Med. 2000 Apr.; 6(4):443-6, 2000, herein incorporated by reference). Therefore, a weekly dose range of 10 μg/g, 5 μg/g, 1 μg/g, 0.5 μg/g, and 0 μg/g may be tested. The steady state plasma level at which tumor growth rate is inhibited by 50% may be graphically determined by the relationship between steady state plasma level and effectiveness. The steady state plasma level may be calculated as described above. If necessary, T may be adjusted accordingly for each modified Fc region polypeptide and the Fc wild type depending on their pharmacokinetic properties to achieve comparable steady state plasma level as RITUXAN. Statistical improved pharmakodynamic values of the modified polypeptide in comparison to the parental polypeptide (e.g. Fc wild type) and RITUXAN will generally indicate that the modified polypeptide confers improved activity in vivo.

In further embodiments, the modified Fc regions of the present invention are screened such that variants that are useful for therapeutic use in at least two species are identified. Such variants are referred to herein as “dual-species improved variants,” and are particularly useful for identifying variants that are therapeutic in humans, and also demonstrate (or are likely to demonstrate) efficacy in an animal model. In this regard, the present invention provides methods for identifying variants that have a strong chance of being approved for human clinical testing since animal model data will likely support any human testing applications made to governmental regulatory agencies (e.g. U.S. Food and Drug Administration).

In certain embodiments, dual-species improved modified polypeptides are identified by first performing an ADCC assay using human effector cells to find improved modified polypeptides, and then performing a second ADCC assay using mouse, rat, or non-human primate effector cells to identify a sub-set of the improved modified polypeptides that are dual-species improved modified polypeptides. In some embodiments, the present invention provides methods for identifying dual-species improved modified polypeptides, comprising: a) providing: i) target cells, ii) a composition comprising a candidate modified polypeptide of a parent polypeptide having at least a portion of an Fc region, wherein the candidate modified polypeptide comprises at least one amino acid modification in the Fc region, and wherein the candidate modified polypeptide mediates target cell cytotoxicity in the presence of a first species (e.g. human) of effector cells more effectively than the parent polypeptide, and iii) second species (e.g. mouse, rat, or non-human primate) effector cells, and b) incubating the composition with the target cells under conditions such that the candidate modified polypeptide binds the target cells thereby generating candidate modified polypeptide bound target cells, c) mixing the second species effector cells with the candidate modified polypeptide bound target cells, and d) measuring target cell cytotoxicity mediated by the candidate modified polypeptide. In certain embodiments, the method further comprises step e) determining if the candidate modified polypeptide mediates target cell cytotoxicity in the presence of the second species effector cells more effectively than the parent polypeptide. In some embodiments, the method further comprises step f) identifying a candidate modified polypeptide as a dual-species improved modified polypeptide that mediates target cell cytotoxicity in the presence of the second species effector cells more effectively than the parent polypeptide. In preferred embodiments, the dual-species modified polypeptides identified are then screened in vivo in one or more animal assays.

In certain embodiments, dual-species improved modified polypeptides are identified by performing any of the assays above using human components (e.g. human cells, human Fc receptors, etc.) to identify improved polypeptides having modified Fc regions, and then running the same assay (or a different assay) with non-human animal components (e.g. mouse cells, mouse Fc receptors, etc.). In this regard, a sub-set of modified polypeptides that perform well according to a given criteria in both human based assays and a second species based assays can be identified.

An exemplary process for identifying dual-species improved polypeptides having modified Fc regions of the invention is a follows. First, a nucleic acid sequence encoding at least a portion of an IgG Fc region is modified such that the amino acid sequence expressed has at least one amino acid change, thereby generating a modified Fc region. This expressed IgG variant is then captured via antigen on an assay plate. Next, the captured variant is screened for soluble human FcγRIII binding using ELISA. If the variant demonstrates improved or comparable (compared to a non-mutated parental Fc region) FcγRIII binding, then the variant is screened for human FcγRIII binding using ELISA. The relative specificity ratio for the variant may then be calculated. Next, an ADCC assay is performed with the variant using human PBMCs or a subset (NK cells or macrophages, for example). If enhanced ADCC activity is found, then the variant is screened in a second ADCC assay using mouse or rat PBMCs. Alternatively, or in addition, an assay can be performed with the variant for binding to cloned rodent receptors or cell lines. Finally, if the variant is found to be improved in the second assay, making it a dual-improved variant, then the variant is screened in vivo in mice or rats. Exemplary Polypeptides Comprising the Modified Fc Regions of the Invention

The variant Fc regions of the present invention may be part of larger molecules, preferably antigen binding molecules (ABMs). The larger molecules may be, for example, monoclonal antibodies, polyclonal antibodies, chimeric antibodies, humanized antibodies, bispecific antibodies, immunoadhesins, etc. As such, it is evident that there is a broad range of applications for the modified Fc regions of the present invention.

For all positions discussed in the present invention, numbering of an immunoglobulin heavy chain is according to the EU index (Kabat et al., 1991, Sequences of Proteins of Immunological Interest, 5th Ed., United States Public Health Service, National Institutes of Health, Bethesda). The “EU index as in Kabat” refers to the residue numbering of the human IgG1 EU antibody.

The antigen binding molecules comprising the modified Fc regions of the present invention may be optimized for a variety of properties. Properties that may be optimized include, but are not limited to, enhanced or reduced affinity for an FcγR. In a preferred embodiment, the modified Fc regions of the present invention are optimized to possess enhanced affinity for a human activating FcγR, preferably FcRI, FcγRIIa, FcγRIIc, FcγRIIIa, and FcγRIIIb, most preferably FcγRIIIa. In an alternately preferred embodiment, the modified Fc regions are optimized to possess reduced affinity for the human inhibitory receptor FcγRIIb. The ABMs of the invention provide antibodies and Fc fusions with enhanced therapeutic properties in humans, for example enhanced effector function and greater anti-cancer potency. In an alternate embodiment, the modified Fc regions of the present invention are optimized to have reduced or ablated affinity for a human FcγR, including but not limited to FcγRI, FcγRIIa, FcγRIIb, or FcγRIIc. These ABMs of the invention are anticipated to provide antibodies and Fc fusions with enhanced therapeutic properties in humans, for example reduced effector function and reduced toxicity. Preferred embodiments comprise optimization of Fc binding to a human FcγR; however, in alternate embodiments, the Fc variants of the present invention possess enhanced or reduced affinity for FcγRs from nonhuman organisms, including but not limited to mice, rats, rabbits, and monkeys. Fc variants that are optimized for binding to a nonhuman FcγR may find use in experimentation. For example, mouse models are available for a variety of diseases that enable testing of properties such as efficacy, toxicity, and pharmacokinetics for a given drug candidate. As is known in the art, cancer cells can be grafted or injected into mice to mimic a human cancer, a process referred to as xenografting. Testing of antibodies or Fc fusions that comprise modified Fc regions that are optimized for one or more mouse FcγRs may provide valuable information with regard to the efficacy of the antibody or Fc fusion, its mechanism of action, and the like.

The modified Fc regions of the present invention may be derived from parent Fc polypeptides that are themselves from a wide range of sources. The parent Fc polypeptide may be substantially encoded by one or more Fc genes from any organism, including but not limited to humans, mice, rats, rabbits, camels, llamas, dromedaries, monkeys, preferably mammals, and most preferably humans and mice. In a preferred embodiment, the parent Fc polypeptide comprises an antibody, referred to as the parent antibody. The parent antibody may be fully human, obtained for example using transgenic mice (Bruggemann et al., 1997, Curr Opin Biotechnol 8:455-458) or human antibody libraries coupled with selection methods (Griffiths et al., 1998, Curr Opin Biotechnol 9:102-108). The parent antibody need not be naturally occurring. For example, the parent antibody may be an engineered antibody, including but not limited to chimeric antibodies and humanized antibodies (Clark, 2000, Immunol Today 21:397-402). The parent antibody may be an engineered variant of an antibody that is substantially encoded by one or more natural antibody genes. In one embodiment, the parent antibody has been affinity matured, as is known in the art. Alternatively, the antibody has been modified in some other way, for example as described in U.S. Ser. No. 10/339,788, filed on Mar. 3, 2003.

The modified Fc regions of the present invention may be substantially encoded by immunoglobulin genes belonging to any of the antibody classes. In a preferred embodiment, the modified Fc regions of the present invention find use in antibodies or Fc fusions that comprise sequences belonging to the IgG class of antibodies, including IgG1, IgG2, IgG3, or IgG4. In an alternate embodiment, the modified Fc regions of the present invention find use in antibodies or Fc fusions that comprise sequences belonging to the IgA (including subclasses IgA1 and IgA2), IgD, IgE, IgG, or IgM classes of antibodies. The modified Fc regions of the present invention may comprise more than one protein chain. That is, the present invention may find use in an antibody or Fc fusion that is a monomer or an oligomer, including a homo- or hetero-oligomer.

The modified Fc regions of the present invention may be combined with other Fc modifications, including but not limited to modifications that alter effector function or interaction with one or more Fc ligands. Such combination may provide additive, synergistic, or novel properties in the ABMs of the invention. In one embodiment, the modified Fc regions of the present invention may be combined with other known Fc modifications (Duncan et al., 1988, Nature 332:563-564; Lund et al., 1991, J Immunol 147:2657-2662; Lund et al., 1992, Mol Immunol 29:53-59; Alegre et al., 1994, Transplantation 57:1537-1543; Hutchins et al., 1995, Proc Natl Acad Sci USA 92:11980-11984; Jefferis et al., 1995, Immunol Left 44:111-117; Lund et al., 1995, Faseb J9:115-119; Jefferis et al., 1996, Immunol Left 54:101-104; Lund et al., 1996, J Immunol 157:4963-4969; Armour et al., 1999, Eur J Immunol 29:2613-2624; Idusogie et al., 2000, J Immunol 164:4178-4184; Reddy et al., 2000, J Immunol 164:1925-1933; Xu et al., 2000, Cell Immunol 200:16-26; Idusogie et al., 2001, J Immunol 166:2571-2575; Shields et al., 2001, J Biol Chem 276:6591-6604; Jefferis et al., 2002, Immunol Left 82:57-65; Presta et al., 2002, Biochem Soc Trans 30:487-490; Hinton et al., 2004, J Biol Chem 279:6213-6216) (U.S. Pat. Nos. 5,624,821; 5,885,573; 6,194,551; PCT WO 00/42072; PCT WO 99/58572; 2004/0002587 A1). Thus, combinations of the modified Fc regions of the present invention with other Fc modifications, as well as undiscovered Fc modifications, are contemplated with the goal of generating novel ABMs (e.g., antibodies or Fc fusions) with optimized properties.

Virtually any antigen may be targeted by ABMs comprising the modified Fc regions of the invention, including but not limited to the following list of proteins, subunits, domains, motifs, and epitopes belonging to the following list of proteins: CD2; CD3, CD3E, CD4, CD11, CD11a, CD14, CD16, CD18, CD19, CD20, CD22, CD23, CD25, CD28, CD29, CD30, CD32, CD33 (p67 protein), CD38, CD40, CD40L, CD52, CD54, CD56, CD80, CD147, GD3, IL-1, IL-1R, IL-2, IL-2R, IL-4, IL-5, IL-6, IL-6R, IL-8, IL-12, IL-15, IL-18, IL-23, interferon α, interferon β, interferon γ; TNF-α, TNFβ2, TNFc, TNFαγ, TNF-RI, TNF-RII, FasL, CD27L, CD30L, 4-1BBL, TRAIL, RANKL, TWEAK, APRIL, BAFF, LIGHT, VEGI, OX40L, TRAIL Receptor-1, A1 Adenosine Receptor, Lymphotoxin Beta Receptor, TACI, BAFF-R, EPO; LFA-3, ICAM-1, ICAM-3, EpCAM, integrin α1, integrin β2, integrin α4/β7, integrin α2, integrin α3, integrin α4, integrin α5, integrin α6, integrin αv, αVβ₃ integrin, FGFR-3, Keratinocyte Growth Factor, VLA-1, VLA-4, L-selectin, anti-Id, E-selectin, HLA, HLA-DR, CTLA-4, T cell receptor, B7-1, B7-2, VNRintegrin, TGFβ1, TGFβ2, eotaxin1, BLyS (B-lymphocyte Stimulator), complement C5, IgE, factor VII, CD64, CBL, NCA 90, EGFR (ErbB-1), Her2/neu (ErbB-2), Her3 (ErbB-3), Her4 (ErbB-4), Tissue Factor, VEGF, VEGFR, endothelin receptor, VLA-4, Hapten NP-cap or NIP-cap, T cell receptor α/β, E-selectin, digoxin, placental alkaline phosphatase (PLAP) and testicular PLAP-like alkaline phosphatase, transferrin receptor, Carcinoembryonic antigen (CEA), CEACAM5, HMFG PEM, mucin MUC1, MUC18, Heparanase I, human cardiac myosin, tumor-associated glycoprotein-72 (TAG-72), tumor-associated antigen CA 125, Prostate specific membrane antigen (PSMA), High molecular weight melanoma-associated antigen (HMW-MAA), carcinoma-associated antigen, Gcoprotein IIb/IIIa (GPIIb/IIIa), tumor-associated antigen expressing Lewis Y related carbohydrate, human cytomegalovirus (HCMV) gH envelope glycoprotein, HIV gp120, HCMV, respiratory syncital virus RSV F, RSVF Fgp, VNRintegrin, IL-8, cytokeratin tumor-associated antigen, Hep B gp120, CMV, gpIIbIIIa, HIV IIIB gp120 V3 loop, respiratory syncytial virus (RSV) Fgp, Herpes simplex virus (HSV) gD glycoprotein, HSV gB glycoprotein, HCMV gB envelope glycoprotein, and Clostridium perfringens toxin.

One of ordinary skill in the art will appreciate that the aforementioned list of targets refers not only to specific proteins and biomolecules, but the biochemical pathway or pathways that comprise them. For example, reference to CTLA-4 as a target antigen implies that the ligands and receptors that make up the T cell co-stimulatory pathway, including CTLA-4, B7-1, B7-2, CD28, and any other undiscovered ligands or receptors that bind these proteins, are also targets. Thus, target as used herein refers not only to a specific biomolecule, but the set of proteins that interact with said target and the members of the biochemical pathway to which said target belongs. One skilled in the art will further appreciate that any of the aforementioned target antigens, the ligands or receptors that bind them, or other members of their corresponding biochemical pathway, may be operably linked to the Fc variants of the present invention in order to generate an Fc fusion. Thus for example, an Fc fusion that targets EGFR could be constructed by operably linking an Fc variant to EGF, TGFα, or any other ligand, discovered or undiscovered, that binds EGFR. Accordingly, a modified Fc region of the present invention could be operably linked to EGFR in order to generate an Fc fusion that binds EGF, TGFα, or any other ligand, discovered or undiscovered, that binds EGFR. Thus, virtually any polypeptide, whether a ligand, receptor, or some other protein or protein domain, including but not limited to the aforementioned targets and the proteins that compose their corresponding biochemical pathways, may be operably linked to the Fc variants of the present invention to develop an Fc fusion.

A number of antibodies and Fc fusions that are approved for use, in clinical trials, or in development may benefit from the modified Fc regions of the present invention. Said antibodies and Fc fusions are herein referred to as “clinical products and candidates.” Thus in a preferred embodiment, the Fc variants of the present invention may find use in a range of clinical products and candidates. For example, a number of antibodies that target CD20 may benefit from the modified Fc regions of the present invention. For example the modified Fc regions of the present invention may find use in an antibody that is substantially similar to rituximab (Rituxan®, IDEC/Genentech/Roche) (see for example U.S. Pat. No. 5,736,137), a chimeric anti-CD20 antibody approved to treat Non-Hodgkin's lymphoma; HuMax-CD20, an anti-CD20 currently being developed by Genmab; an anti-CD20 antibody described in U.S. Pat. No. 5,500,362; AME-133 (Applied Molecular Evolution); hA20 (Immunomedics, Inc.); and HumaLYM (Intracel). A number of antibodies that target members of the family of epidermal growth factor receptors, including EGFR (ErbB-1), Her2/neu (ErbB-2), Her3 (ErbB-3), Her4 (ErbB-4), may benefit from the Fc variants of the present invention. For example the Fc variants of the present invention may find use in an antibody that is substantially similar to trastuzumab (Herceptin®, Genentech) (see for example U.S. Pat. No. 5,677,171), a humanized anti-Her2/neu antibody approved to treat breast cancer; pertuzumab (rhuMab-2C4, Omnitarg.™.), currently being developed by Genentech; an anti-Her2 antibody described in U.S. Pat. No. 4,753,894; cetuximab (Erbitux®), Imclone) (U.S. Pat. No. 4,943,533; PCT WO 96/40210), a chimeric anti-EGFR antibody in clinical trials for a variety of cancers; ABX-EGF (U.S. Pat. No. 6,235,883), currently being developed by Abgenix/Immunex/Amgen; HuMax-EGFr (U.S. Ser. No. 10/172,317), currently being developed by Genmab; 425, EMD55900, EMD62000, and EMD72000 (Merck KGaA) (U.S. Pat. No. 5,558,864; Murthy et al. 1987, Arch Biochem Biophys. 252(2):549-60; Rodeck et al., 1987, J Cell Biochem. 35(4):315-20; Kettleborough et al., 1991, Protein Eng. 4(7):773-83); ICR62 (Institute of Cancer Research) (PCT WO 95/20045; Modjtahedi et al., 1993, J. Cell Biophys. 1993, 22(1-3):129-46; Modjtahedi et al., 1993, Br J Cancer. 1993, 67(2):247-53; Modjtahedi et al, 1996, Br J Cancer, 73(2):228-35; Modjtahedi et al, 2003, Int J Cancer, 105(2):273-80); TheraCIM hR3 (YM Biosciences, Canada and Centro de Immunologia Molecular, Cuba (U.S. Pat. Nos. 5,891,996; 6,506,883; Mateo et al, 1997, Immunotechnology, 3(1):71-81); mAb-806 (Ludwig Institute for Cancer Research, Memorial Sloan-Kettering) (Jungbluth et al. 2003, Proc Natl Acad Sci USA. 100(2):639-44); KSB-102 (KS Biomedix); MR1-1 (IVAX, National Cancer Institute) (PCT WO 0162931A2); and SC100 (Scancell) (PCT WO 01/88138). In another embodiment, the modified Fc regions of the present invention may find use in alemtuzumab (Campath®, Millenium), a humanized monoclonal antibody currently approved for treatment of B-cell chronic lymphocytic leukemia. The modified Fc regions may find use in a variety of antibodies or Fc fusions that are substantially similar to other clinical products and candidates, including but not limited to muromonab-CD3 (Orthoclone OKT3®)), an anti-CD3 antibody developed by Ortho Biotech/Johnson & Johnson, ibritumomab tiuxetan (Zevalin®), an anti-CD20 antibody developed by IDEC/Schering AG, gemtuzumab ozogamicin (Mylotarg®), an anti-CD33 (p67 protein) antibody developed by Celltech/Wyeth, alefacept (Amevive®), an anti-LFA-3 Fc fusion developed by Biogen), abciximab (ReoPro®)), developed by Centocor/Lilly, basiliximab (Simulect®)), developed by Novartis, palivizumab (Synagis®)), developed by MedImmune, infliximab (Remicade®)), an anti-TNFalpha antibody developed by Centocor, adalimumab (Humira®), an anti-TNFalpha antibody developed by Abbott, Humicade®, an anti-TNFalpha antibody developed by Celltech, etanercept (Enbrel®), an anti-TNFalpha Fc fusion developed by Immunex/Amgen, ABX-CBL, an anti-CD147 antibody being developed by Abgenix, ABX-IL8, an anti-IL8 antibody being developed by Abgenix, ABX-MA1, an anti-MUC18 antibody being developed by Abgenix, Pemtumomab (R1549, .sup.90Y-muHMFG1), an anti-MUC1 In development by Antisoma, Therex (R1550), an anti-MUC1 antibody being developed by Antisoma, AngioMab (AS1405), being developed by Antisoma, HuBC-1, being developed by Antisoma, Thioplatin (AS1407) being developed by Antisoma, Antegren® (natalizumab), an anti-alpha-4-beta-1 (VLA-4) and alpha-4-beta-7 antibody being developed by Biogen, VLA-1 mAb, an anti-VLA-1 integrin antibody being developed by Biogen, LTBR mAb, an anti-lymphotoxin beta receptor (LTBR) antibody being developed by Biogen, CAT-152, an anti-TGF.2 antibody being developed by Cambridge Antibody Technology, J695, an anti-IL-12 antibody being developed by Cambridge Antibody Technology and Abbott, CAT-192, an anti-TGF.beta. 1 antibody being developed by Cambridge Antibody Technology and Genzyme, CAT-213, an anti-Eotaxinl antibody being developed by Cambridge Antibody Technology, LymphoStat-B.™. an anti-Blys antibody being developed by Cambridge Antibody Technology and Human Genome Sciences Inc., TRAIL-R1mAb, an anti-TRAIL-R1 antibody being developed by Cambridge Antibody Technology and Human Genome Sciences, Inc., Avastin® (bevacizumab, rhuMAb-VEGF), an anti-VEGF antibody being developed by Genentech, an anti-HER receptor family antibody being developed by Genentech, Anti-Tissue Factor (ATF), an anti-Tissue Factor antibody being developed by Genentech, Xolair® (Omalizumab), an anti-IgE antibody being developed by Genentech, Raptiva®(Efalizumab), an anti-CD11a antibody being developed by Genentech and Xoma, MLN-02 Antibody (formerly LDP-02), being developed by Genentech and Millenium Pharmaceuticals, HuMax CD4, an anti-CD4 antibody being developed by Genmab, HuMax-IL 15, an anti-IL15 antibody being developed by Genmab and Amgen, HuMax-Inflam, being developed by Genmab and Medarex, HuMax-Cancer, an anti-Heparanase I antibody being developed by Genmab and Medarex and Oxford GcoSciences, HuMax-Lymphoma, being developed by Genmab and Amgen, HuMax-TAC, being developed by Genmab, IDEC-131, and anti-CD40L antibody being developed by IDEC Pharmaceuticals, IDEC-151 (Clenoliximab), an anti-CD4 antibody being developed by IDEC Pharmaceuticals, IDEC-114, an anti-CD80 antibody being developed by IDEC Pharmaceuticals, IDEC-152, an anti-CD23 being developed by IDEC Pharmaceuticals, anti-macrophage migration factor (MIF) antibodies being developed by IDEC Pharmaceuticals, BEC2, an anti-idiotypic antibody being developed by Imclone, IMC-1C11, an anti-KDR antibody being developed by Imclone, DC101, an anti-flk-1 antibody being developed by Imclone, anti-VE cadherin antibodies being developed by Imclone, CEA-Cide® (labetuzumab), an anti-carcinoembryonic antigen (CEA) antibody being developed by Immunomedics, LymphoCide® (Epratuzumab), an anti-CD22 antibody being developed by Immunomedics, AFP-Cide, being developed by Immunomedics, MyelomaCide, being developed by Immunomedics, LkoCide, being developed by Immunomedics, ProstaCide, being developed by Immunomedics, MDX-010, an anti-CTLA4 antibody being developed by Medarex, MDX-060, an anti-CD30 antibody being developed by Medarex, MDX-070 being developed by Medarex, MDX-018 being developed by Medarex, Osidem® (IDM-1), and anti-Her2 antibody being developed by Medarex and Immuno-Designed Molecules, HuMax®CD4, an anti-CD4 antibody being developed by Medarex and Genmab, HuMax-IL15, an anti-IL15 antibody being developed by Medarex and Genmab, CNTO 148, an anti-TNFα antibody being developed by Medarex and Centocor/J&J, CNTO 1275, an anti-cytokine antibody being developed by Centocor/J&J, MOR101 and MOR102, anti-intercellular adhesion molecule-1 (ICAM-1) (CD54) antibodies being developed by MorphoSys, MOR201, an anti-fibroblast growth factor receptor 3 (FGFR-3) antibody being developed by MorphoSys, Nuvion® (visilizumab), an anti-CD3 antibody being developed by Protein Design Labs, HuZAF®, an anti-gamma interferon antibody being developed by Protein Design Labs, Anti-.quadrature.5.quadrature.1 Integrin, being developed by Protein Design Labs, anti-IL-12, being developed by Protein Design Labs, ING-1, an anti-Ep-CAM antibody being developed by Xoma, and MLN01, an anti-Beta2 integrin antibody being developed by Xoma.

Application of the modified Fc regions to the aforementioned antibody and Fc fusion clinical products and candidates is not meant to be constrained to their precise composition. The modified Fc regions of the present invention may be incorporated into the aforementioned clinical candidates and products, or into antibodies and Fc fusions that are substantially similar to them. The modified Fc regions of the present invention may be incorporated into versions of the aforementioned clinical candidates and products that are humanized, affinity matured, engineered, or modified in some other way. Furthermore, the entire polypeptide of the aforementioned clinical products and candidates need not be used to construct a new antibody or Fc fusion that incorporates the modified Fc region of the present invention; for example only the variable region of a clinical product or candidate antibody, a substantially similar variable region, or a humanized, affinity matured, engineered, or modified version of the variable region may be used. In another embodiment, the modified Fc region of the present invention may find use in an antibody or Fc fusion that binds to the same epitope, antigen, ligand, or receptor as one of the aforementioned clinical products and candidates.

The modified Fc regions of the present invention may find use in a wide range of antibody and Fc fusion products. In one embodiment, the ABM of the present invention is a therapeutic, a diagnostic, or a research reagent, preferably a therapeutic.

Diseases and disorders capable of being treated or ameliorated by the ABM of the invention include, but are not limited to, autoimmune diseases, immunological diseases, infectious diseases, inflammatory diseases, neurological diseases, and oncological and neoplastic diseases including cancer. By “cancer” and “cancerous” herein refer to or describe the physiological condition in mammals that is typically characterized by unregulated cell growth. Examples of cancer include but are not limited to carcinoma, lymphoma, blastoma, sarcoma (including liposarcoma), neuroendocrine tumors, mesothelioma, schwanoma, meningioma, adenocarcinoma, melanoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophagael cancer, tumors of the biliary tract, as well as head and neck cancer. Furthermore, the Fc variants of the present invention may be used to treat conditions including but not limited to congestive heart failure (CHF), vasculitis, rosecea, acne, eczema, myocarditis and other conditions of the myocardium, systemic lupus erythematosus, diabetes, spondylopathies, synovial fibroblasts, and bone marrow stroma; bone loss; Paget's disease, osteoclastoma; multiple myeloma; breast cancer; disuse osteopenia; malnutrition, periodontal disease, Gaucher's disease, Langerhans' cell histiocytosis, spinal cord injury, acute septic arthritis, osteomalacia, Cushing's syndrome, monoostotic fibrous dysplasia, polyostotic fibrous dysplasia, periodontal reconstruction, and bone fractures; sarcoidosis; multiple myeloma; osteolytic bone cancers, breast cancer, lung cancer, kidney cancer and rectal cancer; bone metastasis, bone pain management, and humoral malignant hypercalcemia, ankylosing spondylitisa and other spondyloarthropathies; transplantation rejection, viral infections, hematologic neoplasisas and neoplastic-like conditions for example, Hodgkin's lymphoma; non-Hodgkin's lymphomas (Burkitt's lymphoma, small lymphocytic lymphoma/chronic lymphocytic leukemia, mycosis fungoides, mantle cell lymphoma, follicular lymphoma, diffuse large B-cell lymphoma, marginal zone lymphoma, hairy cell leukemia and lymphoplasmacytic leukemia), tumors of lymphocyte precursor cells, including B-cell acute lymphoblastic leukemia/lymphoma, and T-cell acute lymphoblastic leukemia/lymphoma, thymoma, tumors of the mature T and NK cells, including peripheral T-cell leukemias, adult T-cell leukemia/T-cell lymphomas and large granular lymphocytic leukemia, Langerhans cell histocytosis, myeloid neoplasias such as acute myelogenous leukemias, including AML with maturation, AML without differentiation, acute promyelocytic leukemia, acute myelomonocytic leukemia, and acute monocytic leukemias, myelodysplastic syndromes, and chronic myeloproliferative disorders, including chronic myelogenous leukemia, tumors of the central nervous system, e.g., brain tumors (glioma, neuroblastoma, astrocytoma, medulloblastoma, ependymoma, and retinoblastoma), solid tumors (nasopharyngeal cancer, basal cell carcinoma, pancreatic cancer, cancer of the bile duct, Kaposi's sarcoma, testicular cancer, uterine, vaginal or cervical cancers, ovarian cancer, primary liver cancer or endometrial cancer, and tumors of the vascular system (angiosarcoma and hemagiopericytoma), osteoporosis, hepatitis, HIV, AIDS, spondyloarthritis, rheumatoid arthritis, inflammatory bowel diseases (IBD), sepsis and septic shock, Crohn's Disease, psoriasis, schleraderma, graft versus host disease (GVHD), allogenic islet graft rejection, hematologic malignancies, such as multiple myeloma (MM), myelodysplastic syndrome (MDS) and acute myelogenous leukemia (AML), inflammation associated with tumors, peripheral nerve injury or demyelinating diseases.

In one embodiment, an ABM comprising a modified Fc region of the present invention is administered to a patient having a disease involving inappropriate expression of a protein. Within the scope of the present invention this is meant to include diseases and disorders characterized by aberrant proteins, due for example to alterations in the amount of a protein present, the presence of a mutant protein, or both. An overabundance may be due to any cause, including but not limited to overexpression at the molecular level, prolonged or accumulated appearance at the site of action, or increased activity of a protein relative to normal. Included within this definition are diseases and disorders characterized by a reduction of a protein. This reduction may be due to any cause, including but not limited to reduced expression at the molecular level, shortened or reduced appearance at the site of action, mutant forms of a protein, or decreased activity of a protein relative to normal. Such an overabundance or reduction of a protein can be measured relative to normal expression, appearance, or activity of a protein, and said measurement may play an important role in the development and/or clinical testing of the ABMs of the present invention.

Engineering Methods

The present invention provides engineering methods that may be used to generate Fc variants. A principal obstacle that has hindered previous attempts at Fc engineering is that only random attempts at modification have been possible, due in part to the inefficiency of engineering strategies and methods, and to the low-throughput nature of antibody production and screening. The present invention describes engineering methods that overcome these shortcomings. A variety of design strategies, computational screening methods, library generation methods, and experimental production and screening methods are contemplated. These strategies, approaches, techniques, and methods may be applied individually or in various combinations to engineer optimized Fc variants.

Design Strategies

One design strategy for engineering Fc variants is provided in which interaction of Fc with some Fc ligand is altered by engineering amino acid modifications at the interface between Fc and said Fc ligand. Fc ligands herein may include but are not limited to FcγRs, Clq, FcRn, protein A or G, and the like. By exploring energetically favorable substitutions at Fc positions that impact the binding interface, variants can be engineered that sample new interface conformations, some of which may improve binding to the Fc ligand, some of which may reduce Fc ligand binding, and some of which may have other favorable properties. Such new interface conformations could be the result of, for example, direct interaction with Fc ligand residues that form the interface, or indirect effects caused by the amino acid modifications such as perturbation of side chain or backbone conformations. Variable positions may be chosen as any positions that are believed to play an important role in determining the conformation of the interface. For example, variable positions may be chosen as the set of residues that are within a certain distance, for example 5 Angstroms, preferably between 1 and 10 Angstroms, of any residue that makes direct contact with the Fc ligand.

An additional design strategy for generating Fc variants is provided in which the conformation of the Fc carbohydrate at N297 is optimized. Optimization as used in this context is meant to include conformational and compositional changes in the N297 carbohydrate that result in a desired property, for example increased or reduced affinity for an FcγR. Such a strategy is supported by the observation that the carbohydrate structure and conformation dramatically affect Fc/FcγR and Fc/Clq binding (Umaña et al., 1999, Nat Biotechnol 17:176-180; Davies et al., 2001, Biotechnol Bioeng 74:288-294; Mimura et al., 2001, J Biol Chem 276:45539-45547.; Radaev et al., 2001, 276 J Biol Chem:16478-16483; Shields et al., 2002, J Biol Chem 277:26733-26740; Shinkawa et al., 2003, J Biol Chem 278:3466-3473). By exploring energetically favorable substitutions at positions that interact with carbohydrate, a quality diversity of variants can be engineered that sample new carbohydrate conformations, some of which may improve and some of which may reduce binding to one or more Fc ligands. While the majority of mutations near the Fc/carbohydrate interface appear to alter carbohydrate conformation, some mutations have been shown to alter the glycosylation composition (Lund et al., 1996, J Immunol 157:4963-4969; Jefferis et al., 2002, Immunol Lett 82:57-65).

Another design strategy for generating Fc variants is provided in which the angle between the Cγ2 and Cγ3 domains is optimized. Optimization as used in this context is meant to describe conformational changes in the Cγ2-Cγ3 domain angle that result in a desired property, for example increased or reduced affinity for an FcγR. This angle is an important determinant of Fc/FcγR affinity (Radaev et al., 2001, J Biol Chem 276:16478-16483), and a number of mutations distal to the Fc/FcγR interface affect binding potentially by modulating it (Shields et al., J Biol Chem 276:6591-6604 (2001)). By exploring energetically favorable substitutions positions that appear to play a key role in determining the Cγ2-Cγ3 angle and the flexibility of the domains relative to one another, a quality diversity of variants can be designed that sample new angles and levels of flexibility, some of which may be optimized for a desired Fc property.

Another design strategy for generating Fc variants is provided in which Fc is reengineered to eliminate the structural and functional dependence on glycosylation. This design strategy involves the optimization of Fc structure, stability, solubility, and/or Fc function (for example affinity of Fc for one or more Fc ligands) in the absence of the N297 carbohydrate. In one approach, positions that are exposed to solvent in the absence of glycosylation are engineered such that they are stable, structurally consistent with Fc structure, and have no tendency to aggregate. The Cγ2 is the only unpaired Ig domain in the antibody. Thus the N297 carbohydrate covers up the exposed hydrophobic patch that would normally be the interface for a protein-protein interaction with another Ig domain, maintaining the stability and structural integrity of Fc and keeping the Cγ2 domains from aggregating across the central axis. Approaches for optimizing aglycosylated Fc may involve but are not limited to designing amino acid modifications that enhance aglycoslated Fc stability and/or solubility by incorporating polar and/or charged residues that face inward towards the Cγ2-Cγ2 dimer axis, and by designing amino acid modifications that directly enhance the aglycosylated Fc/FcγR interface or the interface of aglycosylated Fc with some other Fc ligand.

An additional design strategy for engineering Fc variants is provided in which the conformation of the Cγ2 domain is optimized. Optimization as used in this context is meant to describe conformational changes in the Cγ2 domain angle that result in a desired property, for example increased or reduced affinity for an FcγR. By exploring energetically favorable substitutions at Cγ2 positions that impact the Cγ2 conformation, a quality diversity of variants can be engineered that sample new Cγ2 conformations, some of which may achieve the design goal. Such new Cγ2 conformations could be the result of, for example, alternate backbone conformations that are sampled by the variant. Variable positions may be chosen as any positions that are believed to play an important role in determining Cγ2 structure, stability, solubility, flexibility, function, and the like. For example, Cγ2 hydrophobic core residues, that is Cγ2 residues that are partially or fully sequestered from solvent, may be reengineered. Alternatively, noncore residues may be considered, or residues that are deemed important for determining backbone structure, stability, or flexibility.

An additional design strategy for Fc optimization is provided in which binding to an FcγR, complement, or some other Fc ligand is altered by modifications that modulate the electrostatic interaction between Fc and said Fc ligand. Such modifications may be thought of as optimization of the global electrostatic character of Fc, and include replacement of neutral amino acids with a charged amino acid, replacement of a charged amino acid with a neutral amino acid, or replacement of a charged amino acid with an amino acid of opposite charge (i.e. charge reversal). Such modifications may be used to effect changes in binding affinity between an Fc and one or more Fc ligands, for example Fc.gamma.Rs. In a preferred embodiment, positions at which electrostatic substitutions might affect binding are selected using one of a variety of well known methods for calculation of electrostatic potentials. In the simplest embodiment, Coulomb's law is used to generate electrostatic potentials as a function of the position in the protein. Additional embodiments include the use of Debye-Huckel scaling to account for ionic strength effects, and more sophisticated embodiments such as Poisson-Boltzmann calculations. Such electrostatic calculations may highlight positions and suggest specific amino acid modifications to achieve the design goal. In some cases, these substitutions may be anticipated to variably affect binding to different Fc ligands, for example to enhance binding to activating FcγRs while decreasing binding affinity to inhibitory FcγRs.

Chimeric mouse/human antibodies have been described. See, for example, Morrison, S. L. et al., PNAS 11:6851-6854 (1984); European Patent Publication No. 173494; Boulianna, G. L, et al., Nature 312:642 (1984); Neubeiger, M. S. et al., Nature 314:268 (1985); European Patent Publication No. 125023; Tan et al., J. Immunol. 135:8564 (1985); Sun, L. K et al., Hybridoma 5(1):517 (1986); Sahagan et al., J. Immunol. 137:1066-1074 (1986). See generally, Muron, Nature 312:597 (1984); Dickson, Genetic Engineering News 5(3) (1985); Marx, Science 229:455 (1985); and Morrison, Science 229:1202-1207 (1985).

In a particularly preferred embodiment, the chimeric ABM of the present invention is a humanized antibody. Methods for humanizing non-human antibodies are known in the art. For example, humanized ABMs of the present invention can be prepared according to the methods of U.S. Pat. No. 5,225,539 to Winter; U.S. Pat. No. 6,180,370 to Queen et al.; U.S. Pat. No. 6,632,927 to Adair et al.; U.S. Pat. Appl. Pub. No. 2003/0039649 to Foote; U.S. Pat. Appl. Pub. No. 2004/0044187 to Sato et al.; or U.S. Pat. Appl. Pub. No. 2005/0033028 to Leung et al., the entire contents of each of which are hereby incorporated by reference. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies. The subject humanized antibodies will generally comprise constant regions of human immunoglobulins, such as IgG1.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims et al., J. Immunol., 151:2296 (1993); Chothia et al., J. Mol. Biol., 196:901 (1987)). Another method of selecting the human framework sequence is to compare the sequence of each individual subregion of the full rodent framework (i.e., FR1, FR2, FR3, and FR4) or some combination of the individual subregions (e.g., FR1 and FR2) against a library of known human variable region sequences that correspond to that framework subregion (e.g., as determined by Kabat numbering), and choose the human sequence for each subregion or combination that is the closest to that of the rodent (Leung U.S. patent application Publication No. 2003/0040606A1, published Feb. 27, 2003) (the entire contents of which are hereby incorporated by reference). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)) (the entire contents of each of which are hereby incorporated by reference).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models can be generated using computer programs familiar to those skilled in the art (e.g. InsightII, accelrys inc (former MSI), or at http://swissmodel.expasy.org/ described by Schwede et al., Nucleic Acids Res. 2003 (13):3381-3385). Inspection of these models permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as maintained affinity for the target antigen(s), is achieved. In general, the hypervariable region residues are directly and most substantially involved in influencing antigen binding.

In one embodiment, the ABMs of the present invention comprise a modified human Fc region. In a specific embodiment, the human constant region is IgG1, as set forth in SEQ ID NOs 1 and 2, and set forth below:

Human IgG1 Constant Region Nucleotide Sequence
(SEQ ID NO:1)
ACCAAGGGCCCATCGGTCTTCCCCCTGGCACCCTCCTCCAAGAGCACCTC
TGGGGGCACAGCGGCCCTGGGCTGCCTGGTCAAGGACTACTTCCCCGAAC
CGGTGACGGTGTCGTGGAACTCAGGCGCCCTGACCAGCGGCGTGCACACC
TTCCCGGCTGTCCTACAGTCCTCAGGACTCTACTCCCTCAGCAGCGTGGT
GACCGTGCCCTCCAGCAGCTTGGGCACCCAGACCTACATCTGCAACGTGA
ATCACAAGCCCAGCAACACCAAGGTGGACAAGAAAGCAGAGCCCAAATCT
TGTGACAAAACTCACACATGCCCACCGTGCCCAGCACCTGAACTCCTGGG
GGGACCGTCAGTCTTCCTCTTCCCCCCAAAACCCAAGGACACCCTCATGA
TCTCCCGGACCCCTGAGGTCACATGCGTGGTGGTGGACGTGAGCCACGAA
GACCCTGAGGTCAAGTTCAACTGGTACGTGGACGGCGTGGAGGTGCATAA
TGCCAAGACAAAGCCGCGGGAGGAGCAGTACAACAGCACGTACCGTGTGG
TCAGCGTCCTCACCGTCCTGCACCAGGACTGGCTGAATGGCAAGGAGTAC
AAGTGCAAGGTCTCCAACAAAGCCCTCCCAGCCCCCATCGAGAAAACCAT
CTCCAAAGCCAAAGGGCAGCCCCGAGAACCACAGGTGTACACCCTGCCCC
CATCCCGGGATGAGCTGACCAAGAACCAGGTCAGCCTGACCTGCCTGGTC
AAAGGCTTCTATCCCAGCGACATCGCCGTGGAGTGGGAGAGCAATGGGCA
GCCGGAGAACAACTACAAGACCACGCCTCCCGTGCTGGACTCCGACGGCT
CCTTCTTCCTCTACAGCAAGCTCACCGTGGACAAGAGCAGGTGGCAGCAG
GGGAACGTCTTCTCATGCTCCGTGATGCATGAGGCTCTGCACAACCACTA
CACGCAGAAGAGCCTCTCCCTGTCTCCGGGTAAATGA

Human IgG1 Constant Region Amino Acid Sequence
(SEQ ID NO:2)
TKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKKAEPKS
CDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQ
GNVFSCSVMHEALHNHYTQKSLSLSPGK

However, variants and isoforms of the native human Fc region are also encompassed by the present invention. For example, variant Fc regions suitable for use in the present invention can be produced according to the methods taught in U.S. Pat. No. 6,737,056 to Presta (Fc region variants with altered effector function due to one or more amino acid modifications); or in U.S. Pat. Appl. Nos. 60/439,498; 60/456,041; 60/514,549; or WO 2004/063351 (variant Fc regions with increased binding affinity due to amino acid modification.); or in U.S. patent Ser. No. 10/672,280 or WO 2004/099249 (Fc variants with altered binding to FcγR due to amino acid modification), the contents of each of which are incorporated herein by reference in their entirety.

In another embodiment, the antigen binding molecules of the present invention are engineered to have enhanced binding affinity according to, for example, the methods disclosed in U.S. Pat. Appl. Pub. No. 2004/0132066 to Balint et al., the entire contents of which are hereby incorporated by reference.

In one embodiment, the antigen binding molecule of the present invention is conjugated to an additional moiety, such as a radiolabel or a toxin. Such conjugated ABMs can be produced by numerous methods that are well known in the art.

A variety of radionuclides are applicable to the present invention and those skilled in the art are credited with the ability to readily determine which radionuclide is most appropriate under a variety of circumstances. For example, ¹³¹iodine is a well known radionuclide used for targeted immunotherapy. However, the clinical usefulness of ¹³¹iodine can be limited by several factors including: eight-day physical half-life; dehalogenation of iodinated antibody both in the blood and at tumor sites; and emission characteristics (eg, large gamma component) which can be suboptimal for localized dose deposition in tumor. With the advent of superior chelating agents, the opportunity for attaching metal chelating groups to proteins has increased the opportunities to utilize other radionuclides such as ¹¹¹indium and ⁹⁰yttrium. ⁹⁰Yttrium provides several benefits for utilization in radioimmunotherapeutic applications: the 64 hour half-life of ⁹⁰yttrium is long enough to allow antibody accumulation by tumor and, unlike eg, ¹³¹iodine, ⁹⁰yttrium is a pure beta emitter of high energy with no accompanying gamma irradiation in its decay, with a range in tissue of 100 to 1000 cell diameters. Furthermore, the minimal amount of penetrating radiation allows for outpatient administration of ⁹⁰yttrium-labeled antibodies. Additionally, internalization of labeled antibody is not required for cell killing, and the local emission of ionizing radiation should be lethal for adjacent tumor cells lacking the target antigen.

With respect to radiolabeled antibodies, therapy therewith can also occur using a single therapy treatment or using multiple treatments. Because of the radionuclide component, it is preferred that prior to treatment, peripheral stem cells (“PSC”) or bone marrow (“BM”) be “harvested” for patients experiencing potentially fatal bone marrow toxicity resulting from radiation. BM and/or PSC are harvested using standard techniques, and then purged and frozen for possible reinfusion. Additionally, it is most preferred that prior to treatment a diagnostic dosimetry study using a diagnostic labeled antibody (eg, using ¹¹¹indium) be conducted on the patient, a purpose of which is to ensure that the therapeutically labeled antibody (eg, using ⁹⁰yttrium) will not become unnecessarily “concentrated” in any normal organ or tissue.

In a preferred embodiment, the present invention is directed to an isolated polynucleotide comprising a sequence that encodes a polypeptide of the invention. The invention is further directed to an isolated nucleic acid comprising a sequence at least 80%, 85%, 90%, 95%, 96%, 97%, 98% or 99% identical to a nucleotide sequence of the invention. In another embodiment, the invention is directed to an isolated nucleic acid comprising a sequence that encodes a polypeptide having an amino acid sequence at least 80%, 85%, 90%, 95%, 96%, 97%, 98% or 99% identical to an amino acid sequence of the invention. The invention also encompasses an isolated nucleic acid comprising a sequence that encodes a polypeptide of the invention having one or more conservative amino acid substitutions.

In another embodiment, the present invention is directed to an expression vector and/or a host cell which comprise one or more isolated polynucleotides of the present invention.

Generally, any type of cultured cell line can be used to express the ABM of the present invention. In a preferred embodiment, HEK293-EBNA cells, CHO cells, BHK cells, NS0 cells, SP2/0 cells, YO myeloma cells, P3X63 mouse myeloma cells, PER cells, PER.C6 cells or hybridoma cells, other mammalian cells, yeast cells, insect cells, or plant cells are used as the background cell line to generate the engineered host cells of the invention.

The therapeutic efficacy of the ABMs of the present invention can be enhanced by producing them in a host cell that further expresses a polynucleotide encoding a polypeptide having glycosyltransferase activity. In a preferred embodiment, the polypeptide is selected from the group consisting of: a polypeptide having β(1,4)-N-acetylglucosaminyltransferase III activity; a polypeptide having α-mannosidase II activity, and a polypeptide having β-(1,4)-galactosyltransferase activity. In one embodiment, the host cell expresses a polypeptide having β(1,4)-N-acetylglucosaminyltransferase III activity. In another embodiment, the host cell expresses a polypeptide having β(1,4)-N-acetylglucosaminyltransferase III activity as well as a polypeptide having α-mannosidase II activity. In yet another embodiment, the host cell expresses a polypeptide having β(1,4)-N-acetylglucosaminyltransferase III activity, a polypeptide having α-mannosidase II activity, and a polypeptide having β-(1,4)-galactosyltransferase activity. The polypeptide will be expressed in an amount sufficient to modify the oligosaccharides in the Fc region of the ABM. Alternatively, the host cell may be engineered to have reduced expression of a glycosyltransferase, such as α(1,6)-fucosyltransferase. In a preferred embodiment, the polypeptide having GnT-III activity is a fusion polypeptide comprising the Golgi localization domain of a Golgi resident polypeptide. In another preferred embodiment, the expression of the ABMs of the present invention in a host cell that expresses a polynucleotide encoding a polypeptide having GnT-III activity results in ABMs with increased Fc receptor binding affinity and increased effector function. Accordingly, in one embodiment, the present invention is directed to a host cell comprising (a) an isolated nucleic acid comprising a sequence encoding a polypeptide having GnT-III activity; and (b) an isolated polynucleotide encoding an ABM of the present invention, such as a chimeric, primatized or humanized antibody. In a preferred embodiment, the polypeptide having GnT-III activity is a fusion polypeptide comprising the catalytic domain of GnT-III and the Golgi localization domain is the localization domain of mannosidase II. Methods for generating such fusion polypeptides and using them to produce antibodies with increased effector functions are disclosed in U.S. Provisional Pat. Appl. No. 60/495,142 and U.S. Pat. Appl. Publ. No. 2004/0241817 A1, the entire contents of each of which are expressly incorporated herein by reference. In a particularly preferred embodiment, the chimeric antibody comprises a human Fc. In another preferred embodiment, the antibody is primatized or humanized.

In an alternative embodiment, the ABMs of the present invention can be enhanced by producing them in a host cell that has been engineered to have reduced, inhibited, or eliminated activity of at least one fucosyltransferase, such as α1,6-core fucosyltransferase.

In one embodiment, one or several polynucleotides encoding an ABM of the present invention may be expressed under the control of a constitutive promoter or, alternately, a regulated expression system. Suitable regulated expression systems include, but are not limited to, a tetracycline-regulated expression system, an ecdysone inducible expression system, a lac-switch expression system, a glucocorticoid-inducible expression system, a temperature-inducible promoter system, and a metallothionein metal-inducible expression system. If several different nucleic acids encoding an ABM of the present invention are comprised within the host cell system, some of them may be expressed under the control of a constitutive promoter, while others are expressed under the control of a regulated promoter. The maximal expression level is considered to be the highest possible level of stable polypeptide expression that does not have a significant adverse effect on cell growth rate, and will be determined using routine experimentation. Expression levels are determined by methods generally known in the art, including Western blot analysis using an antibody specific for the ABM or an antibody specific for a peptide tag fused to the ABM; and Northern blot analysis. In a further alternative, the polynucleotide may be operatively linked to a reporter gene; the expression levels of an ABM of the invention are determined by measuring a signal correlated with the expression level of the reporter gene. The reporter gene may be transcribed together with the nucleic acid(s) encoding said fusion polypeptide as a single mRNA molecule; their respective coding sequences may be linked either by an internal ribosome entry site (IRES) or by a cap-independent translation enhancer (CITE). The reporter gene may be translated together with at least one nucleic acid encoding a chimeric ABM such that a single polypeptide chain is formed. The nucleic acids encoding the ABMs of the present invention may be operatively linked to the reporter gene under the control of a single promoter, such that the nucleic acid encoding the fusion polypeptide and the reporter gene are transcribed into an RNA molecule which is alternatively spliced into two separate messenger RNA (m-RNA) molecules; one of the resulting mRNAs is translated into said reporter protein, and the other is translated into said fusion polypeptide.

Methods which are well known to those skilled in the art can be used to construct expression vectors containing the coding sequence of an ABM of the invention along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING A LABORATORY MANUAL, Cold Spring Harbor Laboratory, N.Y. (1989) and Ausubel et al., CURRENT PROTOCOLS IN MOLECULAR BIOLOGY, Greene Publishing Associates and Wiley Interscience, N.Y (1989).

A variety of host-expression vector systems may be utilized to express the coding sequence of the ABMs of the present invention. Preferably, mammalian cells are used as host cell systems transfected with recombinant plasmid DNA or cosmid DNA expression vectors containing the coding sequence of the protein of interest and the coding sequence of the fusion polypeptide. Most preferably, CHO cells, BHK cells, NS0 cells, SP2/0 cells, YO myeloma cells, P3X63 mouse myeloma cells, PER cells, PER.C6 cells or hybridoma cells, other mammalian cells, yeast cells, insect cells, or plant cells are used as host cell system. Some examples of expression systems and selection methods are described in the following references, and references therein: Borth et al., Biotechnol. Bioen. 71(4):266-73 (2000-2001), in Werner et al., Arzneimittelforschung/Drug Res. 48(8):870-80 (1998), in Andersen and Krummen, Curr. Op. Biotechnol. 13:117-123 (2002), in Chadd and Chamow, Curr. Op. Biotechnol. 12:188-194 (2001), and in Giddings, Curr. Op. Biotechnol. 12: 450-454 (2001). In alternate embodiments, other eukaryotic host cell systems may be used, including yeast cells transformed with recombinant yeast expression vectors containing the coding sequence of an ABM of the present invention, such as the expression systems taught in U.S. Pat. Appl. No. 60/344,169 and WO 03/056914 (methods for producing human-like glycoprotein in a non-human eukaryotic host cell) (the contents of each of which are incorporated by reference in their entirety); insect cell systems infected with recombinant virus expression vectors (e.g., baculovirus) containing the coding sequence of a chimeric ABM of the invention; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the coding sequence of the ABM of the invention, including, but not limited to, the expression systems taught in U.S. Pat. No. 6,815,184 (methods for expression and secretion of biologically active polypeptides from genetically engineered duckweed); WO 2004/057002 (production of glycosylated proteins in bryophyte plant cells by introduction of a glycosyl transferase gene) and WO 2004/024927 (methods of generating extracellular heterologous non-plant protein in moss protoplast); and U.S. Pat. Appl. Nos. 60/365,769, 60/368,047, and WO 2003/078614 (glycoprotein processing in transgenic plants comprising a functional mammalian GnTIII enzyme) (the contents of each of which are hereby incorporated by reference in their entirety); or animal cell systems infected with recombinant virus expression vectors (e.g., adenovirus, vaccinia virus) including cell lines engineered to contain multiple copies of the DNA encoding a chimeric ABM of the invention either stably amplified (CHO/dhfr) or unstably amplified in double-minute chromosomes (e.g., murine cell lines). In one embodiment, the vector comprising the polynucleotide(s) encoding the ABM of the invention is polycistronic. Also, in one embodiment, the ABM discussed above is an antibody or a fragment thereof. In a preferred embodiment, the ABM is a humanized antibody.

For the methods of this invention, stable expression is generally preferred to transient expression because it typically achieves more reproducible results and also is more amenable to large-scale production, although transient expression is also encompassed by the invention. Rather than using expression vectors which contain viral origins of replication, host cells can be transformed with the respective coding nucleic acids controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched media, and then are switched to a selective media. The selectable marker in the recombinant plasmid confers resistance to the selection and allows selection of cells which have stably integrated the plasmid into their chromosomes and grow to form foci which in turn can be cloned and expanded into cell lines.

A number of selection systems may be used, including, but not limited to, the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:2026 (1962)), and adenine phosphoribosyltransferase (Lowy et al., Cell 22:817 (1980)) genes, which can be employed in tk-, hgprt- or aprt-cells, respectively. Also, antimetabolite resistance can be used as the basis of selection for dhfr, which confers resistance to methotrexate (Wigler et al., Natl. Acad. Sci. USA 77:3567 (1989); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 (Colberre-Garapin et al., J. Mol. Biol. 150:1 (1981)); and hygro, which confers resistance to hygromycin (Santerre et al., Gene 30:147 (1984) genes. Additional selectable genes have been described, namely trpB, which allows cells to utilize indole in place of tryptophan; hisD, which allows cells to utilize histinol in place of histidine (Hartman & Mulligan, Proc. Natl. Acad. Sci. USA 85:8047 (1988)); the glutamine synthase system; and ODC (ornithine decarboxylase) which confers resistance to the ornithine decarboxylase inhibitor, 2-(difluoromethyl)-DL-ornithine, DFMO (McConlogue, in: Current Communications in Molecular Biology, Cold Spring Harbor Laboratory ed. (1987)).

The present invention is further directed to a method for modifying the glycosylation profile of the ABMs of the present invention that are produced by a host cell, comprising expressing in said host cell a nucleic acid encoding an ABM of the invention and a nucleic acid encoding a polypeptide with glycosyltransferase activity or a vector comprising such nucleic acids. In a preferred embodiment, the polypeptide is selected from the group consisting of: a polypeptide having β(1,4)-N-acetylglucosaminyltransferase III activity; a polypeptide having α-mannosidase II activity, and a polypeptide having β-(1,4)-galactosyltransferase activity. In one embodiment, the host cell expresses a polypeptide having β(1,4)-N-acetylglucosaminyltransferase III activity. In another embodiment, the host cell expresses a polypeptide having β(1,4)-N-acetylglucosaminyltransferase III activity as well as a polypeptide having α-mannosidase II activity. In yet another embodiment, the host cell expresses a polypeptide having β(1,4)-N-acetylglucosaminyltransferase III activity, a polypeptide having α-mannosidase II activity, and a polypeptide having β-(1,4)-galactosyltransferase. Preferably, the modified polypeptide is IgG or a fragment thereof comprising the Fc region. In a particularly preferred embodiment the ABM is a humanized antibody or a fragment thereof. Alternatively, or in addition, such host cells may be engineered to have reduced, inhibited, or eliminated activity of at least one fucosyltransferase. In another embodiment, the host cell is engineered to coexpress an ABM of the invention, GnT-III and mannosidase II (ManII).

The modified ABMs produced by the host cells of the invention exhibit increased Fc receptor binding affinity and/or increased effector function as a result of the modification. In a particularly preferred embodiment the ABM is a humanized antibody or a fragment thereof containing the Fc region. Preferably, the increased Fc receptor binding affinity is increased binding to a Fcγ activating receptor, such as the FcγRIIIa receptor. The increased effector function is preferably an increase in one or more of the following: increased antibody-dependent cellular cytotoxicity, increased antibody-dependent cellular phagocytosis (ADCP), increased cytokine secretion, increased immune-complex-mediated antigen uptake by antigen-presenting cells, increased Fc-mediated cellular cytotoxicity, increased binding to NK cells, increased binding to macrophages, increased binding to polymorphonuclear cells (PMNs), increased binding to monocytes, increased crosslinking of target-bound antibodies, increased direct signaling inducing apoptosis, increased dendritic cell maturation, or increased T cell priming.

The present invention is also directed to a method for producing an ABM of the present invention, having modified oligosaccharides in a host cell comprising (a) culturing a host cell engineered to express at least one nucleic acid encoding a polypeptide having glycosyltransferase activity under conditions which permit the production of an ABM according to the present invention, wherein said polypeptide having glycosyltransferase activity is expressed in an amount sufficient to modify the oligosaccharides in the Fc region of said ABM produced by said host cell; and (b) isolating said ABM. In a preferred embodiment, the polypeptide is selected from the group consisting of: a polypeptide having β(1,4)-N-acetylglucosaminyltransferase III activity; a polypeptide having α-mannosidase II activity, and a polypeptide having β-(1,4)-galactosyltransferase activity. In one embodiment, the host cell expresses a polypeptide having β(1,4)-N-acetylglucosaminyltransferase III activity. In another embodiment, the host cell expresses a polypeptide having β(1,4)-N-acetylglucosaminyltransferase III activity as well as a polypeptide having α-mannosidase II activity. In yet another embodiment, the host cell expresses a polypeptide having β(1,4)-N-acetylglucosaminyltransferase III activity, a polypeptide having α-mannosidase II activity, and a polypeptide having β-(1,4)-galactosyltransferase In a preferred embodiment, the polypeptide having GnT-III activity is a fusion polypeptide comprising the catalytic domain of GnT-III. In a particularly preferred embodiment, the fusion polypeptide further comprises the Golgi localization domain of a Golgi resident polypeptide.

Preferably, the Golgi localization domain is the localization domain of mannosidase II or GnT-I. Alternatively, the Golgi localization domain is selected from the group consisting of: the localization domain of mannosidase I, the localization domain of GnT-II, and the localization domain of a 1-6 core fucosyltransferase. The ABMs produced by the methods of the present invention have increased Fc receptor binding affinity and/or increased effector function. Preferably, the increased effector function is one or more of the following: increased Fc-mediated cellular cytotoxicity (including increased antibody-dependent cellular cytotoxicity), increased antibody-dependent-cellular phagocytosis (ADCP), increased cytokine secretion, increased immune-complex-mediated antigen uptake by antigen-presenting cells, increased binding to NK cells, increased binding to macrophages, increased binding to monocytes, increased binding to polymorphonuclear cells, increased direct signaling inducing apoptosis, increased crosslinking of target-bound antibodies, increased dendritic cell maturation, or increased T cell priming. The increased Fc receptor binding affinity is preferably increased binding to Fc activating receptors such as FcγRIIa. In a particularly preferred embodiment the ABM is a humanized antibody or a fragment thereof.

In another embodiment, the present invention is directed to a chimeric ABM having a modified Fc region and which has an increased proportion of bisected oligosaccharides in the Fc region of said polypeptide. It is contemplated that such an ABM encompasses antibodies and fragments thereof comprising the Fc region. In a preferred embodiment, the ABM is a humanized antibody. In one embodiment, the percentage of bisected oligosaccharides in the Fc region of the ABM is at least 50%, more preferably, at least 60%, at least 70%, at least 80%, or at least 90%, and most preferably at least 90-95% of the total oligosaccharides. In yet another embodiment, the ABM produced by the methods of the invention has an increased proportion of nonfucosylated oligosaccharides in the Fc region as a result of the modification of its oligosaccharides by the methods of the present invention. In one embodiment, the percentage of nonfucosylated oligosaccharides is at least 50%, preferably, at least 60% to 70%, most preferably at least 75%. The nonfucosylated oligosaccharides may be of the hybrid or complex type. In a particularly preferred embodiment, the ABM produced by the host cells and methods of the invention has an increased proportion of bisected, nonfucosylated oligosaccharides in the Fc region. The bisected, nonfucosylated oligosaccharides may be either hybrid or complex. Specifically, the methods of the present invention may be used to produce ABMs in which at least 15%, more preferably at least 20%, more preferably at least 25%, more preferably at least 30%, more preferably at least 35% of the oligosaccharides in the Fc region of the ABM are bisected, nonfucosylated. The methods of the present invention may also be used to produce polypeptides in which at least 15%, more preferably at least 20%, more preferably at least 25%, more preferably at least 30%, more preferably at least 35% of the oligosaccharides in the Fc region of the polypeptide are bisected hybrid nonfucosylated.

In another embodiment, the present invention is directed to a chimeric ABM having a modified Fc region and engineered to have increased effector function and/or increased Fc receptor binding affinity, produced by the methods of the invention. Preferably, the increased effector function is one or more of the following: increased Fc-mediated cellular cytotoxicity (including increased antibody-dependent cellular cytotoxicity), increased antibody-dependent cellular phagocytosis (ADCP), increased cytokine secretion, increased immune-complex-mediated antigen uptake by antigen-presenting cells, increased binding to NK cells, increased binding to macrophages, increased binding to monocytes, increased binding to polymorphonuclear cells, increased direct signaling inducing apoptosis, increased crosslinking of target-bound antibodies, increased dendritic cell maturation, or increased T cell priming. In a preferred embodiment, the increased Fc receptor binding affinity is increased binding to a Fc activating receptor, most preferably FcγRIIIa. In one embodiment, the ABM is an antibody, an antibody fragment containing the Fc region, or a fusion protein that includes a region equivalent to the Fc region of an immunoglobulin. In a particularly preferred embodiment, the ABM is a humanized antibody.

The present invention is further directed to pharmaceutical compositions comprising the ABMs of the present invention and a pharmaceutically acceptable carrier.

The present invention is further directed to the use of such pharmaceutical compositions in the method of treatment of cancer. Specifically, the present invention is directed to a method for the treatment or prophylaxis of cancer comprising administering a therapeutically effective amount of the pharmaceutical composition of the invention.

The present invention is further directed to the use of such pharmaceutical compositions in the method of treatment of a precancerous condition or lesion. Specifically, the present invention is directed to a method for the treatment or prophylaxis of a precancerous condition or lesion comprising administering a therapeutically effective amount of the pharmaceutical composition of the invention.

The present invention further provides methods for the generation and use of host cell systems for the production of glycoforms of the ABMs of the present invention, having increased Fc receptor binding affinity, preferably increased binding to Fc activating receptors, and/or having increased effector functions, including antibody-dependent cellular cytotoxicity. The glycoengineering methodology that can be used with the ABMs of the present invention has been described in greater detail in U.S. Pat. No. 6,602,684, U.S. Pat. Appl. Publ. No. 2004/0241817 A1, U.S. Pat. Appl. Publ. No. 2003/0175884 A1, Provisional U.S. Patent Application No. 60/441,307 and WO 2004/065540, the entire contents of each of which are incorporated herein by reference in its entirety. The ABMs of the present invention can alternatively be glycoengineered to have reduced fucose residues in the Fc region according to the techniques disclosed in U.S. Pat. Appl. Pub. No. 2003/0157108 (Genentech) or in EP 1 176 195 A1, WO 03/084570, WO 03/085119 and U.S. Pat. Appl. Pub. Nos. 2003/0115614, 2004/093621, 2004/110282, 2004/110704, 2004/132140 (all to Kyowa Hakko Kogyo Ltd.). The contents of each of these documents are hereby incorporated by reference in their entirety. Glycoengineered ABMs of the invention may also be produced in expression systems that produce modified glycoproteins, such as those taught in U.S. Pat. Appl. Pub. No. 60/344,169 and WO 03/056914 (GlycoFi, Inc.) or in WO 2004/057002 and WO 2004/024927 (Greenovation), the contents of each of which are hereby incorporated by reference in their entirety.

Generation of Cell Lines for the Production of Proteins with Altered Glycosylation Pattern

The present invention provides host cell expression systems for the generation of the ABMs of the present invention having modified Fc regions and modified Fc glycosylation patterns. In particular, the present invention provides host cell systems for the generation of glycoforms of the ABMs of the present invention having an improved therapeutic value. Therefore, the invention provides host cell expression systems selected or engineered to express a polypeptide having GnT-III activity. In one embodiment, the polypeptide having GnT-III activity is a fusion polypeptide comprising the Golgi localization domain of a heterologous Golgi resident polypeptide. Specifically, such host cell expression systems may be engineered to comprise a recombinant nucleic acid molecule encoding a polypeptide having GnT-III, operatively linked to a constitutive or regulated promoter system.

In one specific embodiment, the present invention provides a host cell that has been engineered to express at least one nucleic acid encoding a fusion polypeptide having GnT-III activity and comprising the Golgi localization domain of a heterologous Golgi resident polypeptide. In one aspect, the host cell is engineered with a nucleic acid molecule comprising at least one gene encoding a fusion polypeptide having GnT-III activity and comprising the Golgi localization domain of a heterologous Golgi resident polypeptide.

Generally, any type of cultured cell line, including the cell lines discussed above, can be used as a background to engineer the host cell lines of the present invention. In a preferred embodiment, CHO cells, BHK cells, NS0 cells, SP2/0 cells, YO myeloma cells, P3X63 mouse myeloma cells, PER cells, PER.C6 cells or hybridoma cells, other mammalian cells, yeast cells, insect cells, or plant cells are used as the background cell line to generate the engineered host cells of the invention.

The invention is contemplated to encompass any engineered host cells expressing a polypeptide having GnT-III activity, including a fusion polypeptide that comprises the Golgi localization domain of a heterologous Golgi resident polypeptide as defined herein.

One or several nucleic acids encoding a polypeptide having GnT-III activity may be expressed under the control of a constitutive promoter or, alternately, a regulated expression system. Such systems are well known in the art, and include the systems discussed above. If several different nucleic acids encoding fusion polypeptides having GnT-III activity and comprising the Golgi localization domain of a heterologous Golgi resident polypeptide are comprised within the host cell system, some of them may be expressed under the control of a constitutive promoter, while others are expressed under the control of a regulated promoter. Expression levels of the fusion polypeptides having GnT-III activity are determined by methods generally known in the art, including Western blot analysis, Northern blot analysis, reporter gene expression analysis or measurement of GnT-III activity. Alternatively, a lectin may be employed which binds to biosynthetic products of the GnT-III, for example, E4-PHA lectin. Alternatively, a functional assay which measures the increased Fc receptor binding or increased effector function mediated by antibodies produced by the cells engineered with the nucleic acid encoding a polypeptide with GnT-III activity may be used.

Identification Of Transfectants Or Transformants that Express the Protein having A Modified Glycosylation Pattern

The host cells which contain the coding sequence of a chimeric ABM and which express the biologically active gene products may be identified by at least four general approaches; (a) DNA-DNA or DNA-RNA hybridization; (b) the presence or absence of “marker” gene functions; (c) assessing the level of transcription as measured by the expression of the respective mRNA transcripts in the host cell; and (d) detection of the gene product as measured by immunoassay or by its biological activity.

In the first approach, the presence of the coding sequence of a chimeric ABM of the invention and the coding sequence of the polypeptide having GnT-III activity can be detected by DNA-DNA or DNA-RNA hybridization using probes comprising nucleotide sequences that are homologous to the respective coding sequences, respectively, or portions or derivatives thereof.

In the second approach, the recombinant expression vector/host system can be identified and selected based upon the presence or absence of certain “marker” gene functions (e.g., thymidine kinase activity, resistance to antibiotics, resistance to methotrexate, transformation phenotype, occlusion body formation in baculovirus, etc.). For example, if the coding sequence of the ABM of the invention, or a fragment thereof, and the coding sequence of the polypeptide having GnT-III activity are inserted within a marker gene sequence of the vector, recombinants containing the respective coding sequences can be identified by the absence of the marker gene function. Alternatively, a marker gene can be placed in tandem with the coding sequences under the control of the same or different promoter used to control the expression of the coding sequences. Expression of the marker in response to induction or selection indicates expression of the coding sequence of the ABM of the invention and the coding sequence of the polypeptide having GnT-III activity.

In the third approach, transcriptional activity for the coding region of the ABM of the invention, or a fragment thereof, and the coding sequence of the polypeptide having GnT-III activity can be assessed by hybridization assays. For example, RNA can be isolated and analyzed by Northern blot using a probe homologous to the coding sequences of the ABM of the invention, or a fragment thereof, and the coding sequence of the polypeptide having GnT-III activity or particular portions thereof. Alternatively, total nucleic acids of the host cell may be extracted and assayed for hybridization to such probes.

In the fourth approach, the expression of the protein products can be assessed immunologically, for example by Western blots, immunoassays such as radioimmuno-precipitation, enzyme-linked immunoassays and the like. The ultimate test of the success of the expression system, however, involves the detection of the biologically active gene products.

Generation and Use of ABMs Having Increased Effector Function Including Antibody Dependent Cellular Cytotoxicity

In preferred embodiments, the present invention provides glycoforms of chimeric ABMs having modified Fc regions and having increased effector function including antibody-dependent cellular cytotoxicity. Glycosylation engineering of antibodies has been previously described. See, e.g., U.S. Pat. No. 6,602,684, incorporated herein by reference in its entirety.

Clinical trials of unconjugated monoclonal antibodies (mAbs) for the treatment of some types of cancer have recently yielded encouraging results. Dillman, Cancer Biother. & Radiopharm. 12:223-25 (1997); Deo et al., Immunology Today 18:127 (1997). A chimeric, unconjugated IgG1 has been approved for low-grade or follicular B-cell non-Hodgkin's lymphoma. Dillman, Cancer Biother. & Radiopharm. 12:223-25 (1997), while another unconjugated mAb, a humanized IgG1 targeting solid breast tumors, has also been showing promising results in phase III clinical trials. Deo et al., Immunology Today 18:127 (1997). The antigens of these two mAbs are highly expressed in their respective tumor cells and the antibodies mediate potent tumor destruction by effector cells in vitro and in vivo. In contrast, many other unconjugated mAbs with fine tumor specificities cannot trigger effector functions of sufficient potency to be clinically useful. Frost et al., Cancer 80:317-33 (1997); Surfus et al., J Immunother. 19:184-91 (1996). For some of these weaker mAbs, adjunct cytokine therapy is currently being tested. Addition of cytokines can stimulate antibody-dependent cellular cytotoxicity (ADCC) by increasing the activity and number of circulating lymphocytes. Frost et al., Cancer 80:317-33 (1997); Surfus et al., J Immunother. 19:184-91 (1996). ADCC, a lytic attack on antibody-targeted cells, is triggered upon binding of leukocyte receptors to the constant region (Fc) of antibodies. Deo et al., Immunology Today 18:127 (1997).

A different, but complementary, approach to increase ADCC activity of unconjugated IgG1s is to engineer the Fc region of the antibody. Protein engineering studies have shown that FcγRs interact mainly with the hinge region of the IgG molecule. Lund et al., J. Immunol. 157:4963-69 (1996). However, FcγR binding also requires the presence of oligosaccharides covalently attached at the conserved Asn 297 in the CH2 region. Lund et al., J. Immunol. 157:4963-69 (1996); Wright and Morrison, Trends Biotech. 15:26-31 (1997), suggesting that either oligosaccharide and polypeptide both directly contribute to the interaction site or that the oligosaccharide is required to maintain an active CH2 polypeptide conformation. Modification of the oligosaccharide structure can therefore be explored as a means to increase the affinity of the interaction.

An IgG molecule carries two N-linked oligosaccharides in its Fc region, one on each heavy chain. As any glycoprotein, an antibody is produced as a population of glycoforms which share the same polypeptide backbone but have different oligosaccharides attached to the glycosylation sites. The oligosaccharides normally found in the Fc region of serum IgG are of complex bi-antennary type (Wormald et al., Biochemistry 36:130-38 (1997), with a low level of terminal sialic acid and bisecting N-acetylglucosamine (GlcNAc), and a variable degree of terminal galactosylation and core fucosylation. Some studies suggest that the minimal carbohydrate structure required for FcγR binding lies within the oligosaccharide core. Lund et al., J. Immunol. 157:4963-69 (1996)

The mouse- or hamster-derived cell lines used in industry and academia for production of unconjugated therapeutic mAbs normally attach the required oligosaccharide determinants to Fc sites. IgGs expressed in these cell lines, however, lack the bisecting GlcNAc found in low amounts in serum IgGs. Lifely et al., Glycobiology 318:813-22 (1995). In contrast, it was observed that a rat myeloma-produced, humanized IgG1 (CAMPATH-1H) carried a bisecting GlcNAc in some of its glycoforms. Lifely et al., Glycobiology 318:813-22 (1995). The rat cell-derived antibody reached a similar maximal in vitro ADCC activity as CAMPATH-1H antibodies produced in standard cell lines, but at significantly lower antibody concentrations.

The CAMPATH antigen is normally present at high levels on lymphoma cells, and this chimeric mAb has high ADCC activity in the absence of a bisecting GlcNAc. Lifely et al., Glycobiology 318:813-22 (1995). In the N-linked glycosylation pathway, a bisecting GlcNAc is added by GnT-III. Schachter, Biochem. Cell Biol. 64:163-81 (1986).

Previous studies used a single antibody-producing CHO cell line, that was previously engineered to express, in an externally-regulated fashion, different levels of a cloned GnT-III gene enzyme (Umana, P., et al., Nature Biotechnol. 17:176-180 (1999)). This approach established for the first time a correlation between expression of GnT-III and the ADCC activity of the modified antibody. Thus, the invention contemplates a recombinant, chimeric or humanized ABM (e.g., antibody) or a fragment thereof having a modified Fc region from one or more amino acid modifications and having altered glycosylation resulting from increased GnT-III activity. The increased GnT-III activity results in an increase in the percentage of bisected oligosaccharides, as well as a decrease in the percentage of fucose residues, in the Fc region of the ABM. This antibody, or fragment thereof, has increased Fc receptor binding affinity and increased effector function. In addition, the invention is directed to antibody fragments and fusion proteins comprising a region that is equivalent to the Fc region of immunoglobulins.

Therapeutic Applications of ABMs Produced According to the Methods of the Invention.

In the broadest sense, the ABMs of the present invention can be used to target cells in vivo or in vitro that express a desired antigen. The cells expressing the desired antigen can be targetted for diagnostic or therapeutic purposes. In one aspect, the ABMs of the present invention can be used to detect the presence of the antigen in a sample. In another aspect, the ABMs of the present invention can be used to bind antigen-expressing cells in vitro or in vivo for, e.g., identification or targeting. More particularly, the ABMs of the present invention can be used to block or inhibit antigen binding to an antigen ligand or, alternatively, target an antigen-expressing cell for destruction.

The ABMs of the present invention can be used alone to target and kill tumor cells in vivo. The ABMs can also be used in conjunction with an appropriate therapeutic agent to treat human carcinoma. For example, the ABMs can be used in combination with standard or conventional treatment methods such as chemotherapy, radiation therapy, or can be conjugated or linked to a therapeutic drug, or toxin, as well as to a lymphokine or a tumor inhibitory growth factor, for delivery of the therapeutic agent to the site of the carcinoma. The conjugates of the ABMs of this invention that are of prime importance are (1) immunotoxins (conjugates of the ABM and a cytotoxic moiety) and (2) labeled (e.g. radiolabeled, enzyme-labeled, or fluorochrome-labeled) ABMs in which the label provides a means for identifying immune complexes that include the labeled ABM. The ABMs can also be used to induce lysis through the natural complement process, and to interact with antibody dependent cytotoxic cells normally present.

The cytotoxic moiety of the immunotoxin may be a cytotoxic drug or an enzymatically active toxin of bacterial or plant origin, or an enzymatically active fragment (“A chain”) of such a toxin. Enzymatically active toxins and fragments thereof used are diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolacca americana proteins (PAPI, PAPII, and PAP-S), momordica charantia inhibitor, curcin, crotin, sapaonaria officinalis inhibitor, gelonin, mitogellin, restrictocin, phenomycin, and enomycin. In another embodiment, the ABMs are conjugated to small molecule anticancer drugs. Conjugates of the ABM and such cytotoxic moieties are made using a variety of bifunctional protein coupling agents. Examples of such reagents are SPDP, IT, bifunctional derivatives of imidoesters such a dimethyl adipimidate HCl, active esters such as disuccinimidyl suberate, aldehydes such as glutaraldehyde, bis-azido compounds such as bis (p-azidobenzoyl) hexanediamine, bis-diazonium derivatives such as bis-(p-diazoniumbenzoyl)-ethylenediamine, diisocyanates such as tolylene 2,6-diisocyanate, and bis-active fluorine compounds such as 1,5-difluoro-2,4-dinitrobenzene. The lysing portion of a toxin may be joined to the Fab fragment of the ABMs. Additional appropriate toxins are known in the art, as evidenced in e.g., published U.S. patent application No. 2002/0128448, incorporated herein by reference in its entirety.

In one embodiment, a chimeric, glycoengineered ABM of the invention, is conjugated to ricin A chain. Most advantageously, the ricin A chain is deglycosylated and produced through recombinant means. An advantageous method of making the ricin immunotoxin is described in Vitetta et al., Science 238:1098 (1987), hereby incorporated by reference.

When used to kill human cancer cells in vitro for diagnostic purposes, the conjugates will typically be added to the cell culture medium at a concentration of at least about 10 nM. The formulation and mode of administration for in vitro use are not critical. Aqueous formulations that are compatible with the culture or perfusion medium will normally be used. Cytotoxicity may be read by conventional techniques to determine the presence or degree of cancer.

As discussed above, a cytotoxic radiopharmaceutical for treating cancer may be made by conjugating a radioactive isotope (e.g., I, Y, Pr) to a chimeric, glycoengineered ABM having substantially the same binding specificity of the murine monoclonal antibody. The term “cytotoxic moiety” as used herein is intended to include such isotopes.

In another embodiment, liposomes are filled with a cytotoxic drug and the liposomes are coated with the ABMs of the present invention.

Techniques for conjugating such therapeutic agents to antibodies are well known (see, e.g., Amon et al., “Monoclonal Antibodies for Immunotargeting of Drugs in Cancer Therapy”, in Monoclonal Antibodies and Cancer Therapy, Reisfeld et al. (eds.), pp. 243 56 (Alan R. Liss, Inc. 1985); Hellstrom et al., “Antibodies For Drug Delivery”, in Controlled Drug Delivery (2nd Ed.), Robinson et al. (eds.), pp. 623 53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); and Thorpe et al., “The Preparation And Cytotoxic Properties Of Antibody Toxin Conjugates”, Immunol. Rev. 62:119 58 (1982)).

Still other therapeutic applications for the ABMs of the invention include conjugation or linkage, e.g., by recombinant DNA techniques, to an enzyme capable of converting a prodrug into a cytotoxic drug and the use of that antibody enzyme conjugate in combination with the prodrug to convert the prodrug to a cytotoxic agent at the tumor site (see, e.g., Senter et al., Proc. Natl. Acad. Sci. USA 85:4842-46 (1988); Senter et al., Cancer Research 49:5789-5792 (1989); and Senter, FASEB J 4:188 193 (1990)).

Still another therapeutic use for the ABMs of the invention involves use, either unconjugated, in the presence of complement, or as part of an antibody drug or antibody toxin conjugate, to remove tumor cells from the bone marrow of cancer patients. According to this approach, autologous bone marrow may be purged ex vivo by treatment with the antibody and the marrow infused back into the patient (see, e.g., Ramsay et al., J. Clin. Immunol., 8(2):81 88 (1988)).

Similarly, a fusion protein comprising at least the antigen binding region of an ABM of the invention joined to at least a functionally active portion of a second protein having anti tumor activity, e.g., a lymphokine or oncostatin, can be used to treat human carcinoma in vivo.

The present invention provides a method for selectively killing tumor cells expressing a target antigen. This method comprises reacting the immunoconjugate (e.g., the immunotoxin) of the invention with said tumor cells. These tumor cells may be from a human carcinoma.

Additionally, this invention provides a method of treating carcinomas (for example, human carcinomas) in vivo. This method comprises administering to a subject a pharmaceutically effective amount of a composition containing at least one of the immunoconjugates (e.g., the immunotoxin) of the invention.

In a further aspect, the invention is directed to an improved method for treating cell proliferation disorders wherein a tumor associated antigen is expressed, particularly wherein said tumor associated antigen is abnormally expressed (e.g. overexpressed), comprising administering a therapeutically effective amount of an ABM of the present invention to a human subject in need thereof.

Similarly, other cell proliferation disorders can also be treated by the ABMs of the present invention. Examples of such cell proliferation disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other cell proliferation disease, besides neoplasia, located in an organ system listed above.

In accordance with the practice of this invention, the subject may be a human, equine, porcine, bovine, murine, canine, feline, and avian subjects. Other warm blooded animals are also included in this invention.

The subject invention further provides methods for inhibiting the growth of human tumor cells, treating a tumor in a subject, and treating a proliferative type disease in a subject. These methods comprise administering to the subject an effective amount of an ABM composition of the invention.

It is apparent, therefore, that the present invention encompasses pharmaceutical compositions, combinations, and methods for the treatment or prophylaxis of cancer or for use in the treatment or prophylaxis of a precancerous condition or lesion. The invention includes pharmaceutical compositions for use in the treatment or prophylaxis of human malignancies such as melanomas and cancers of the bladder, brain, head and neck, pancreas, lung, breast, ovary, colon, prostate, and kidney. For example, the invention includes pharmaceutical compositions for use in the treatment or prophylaxis of cancers, such as human malignancies, or for use in the treatment or prophylaxis of a precancerous condition or lesion comprising a pharmaceutically effective amount of an antigen binding molecule of the present invention and a pharmaceutically acceptable carrier. The cancer may be, for example, lung cancer, non small cell lung (NSCL) cancer, bronchioalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. The precancerous condition or lesion includes, for example, the group consisting of oral leukoplakia, actinic keratosis (solar keratosis), precancerous polyps of the colon or rectum, gastric epithelial dysplasia, adenomatous dysplasia, hereditary nonpolyposis colon cancer syndrome (HNPCC), Barrett's esophagus, bladder dysplasia, and precancerous cervical conditions.

Preferably, said cancer is selected from the group consisting of breast cancer, bladder cancer, head & neck cancer, skin cancer, pancreatic cancer, lung cancer, ovarian cancer, colon cancer, prostate cancer, kidney cancer, and brain cancer.

The phrase “pharmaceutically acceptable” is employed herein to refer to those compounds, material, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Any conventional carrier material can be utilized. The carrier material can be an organic or inorganic one suitable for eteral, percutaneous or parenteral administration. Suitable carriers include water, gelatin, gum arabic, lactose, starch, magnesium stearate, talc, vegetable oils, polyalkylene-glycols, petroleum jelly and the like. Furthermore, the pharmaceutical preparations may contain other pharmaceutically active agents. Additional additives such as flavoring agents, stabilizers, emulifying agents, buffers and the like may be added in accordance with accepted practices of pharmaceutical compounding.

In yet another embodiment, the invention relates to an ABM according to the present invention for use as a medicament, in particular for use in the treatment or prophylaxis of cancer or for use in the treatment or prophylaxis of a precancerous condition or lesion. The cancer may be, for example, lung cancer, non small cell lung (NSCL) cancer, bronchioalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. The precancerous condition or lesion includes, for example, the group consisting of oral leukoplakia, actinic keratosis (solar keratosis), precancerous polyps of the colon or rectum, gastric epithelial dysplasia, adenomatous dysplasia, hereditary nonpolyposis colon cancer syndrome (HNPCC), Barrett's esophagus, bladder dysplasia, and precancerous cervical conditions.

Preferably, said cancer is selected from the group consisting of breast cancer, bladder cancer, head & neck cancer, skin cancer, pancreatic cancer, lung cancer, ovarian cancer, colon cancer, prostate cancer, kidney cancer, and brain cancer.

Yet another embodiment is the use of the ABM according to the present invention for the manufacture of a medicament for the treatment or prophylaxis of cancer. Cancer is as defined above.

Preferably, said cancer is selected from the group consisting of breast cancer, bladder cancer, head & neck cancer, skin cancer, pancreatic cancer, lung cancer, ovarian cancer, colon cancer, prostate cancer, kidney cancer, and brain cancer.

Also preferably, said antigen binding molecule is used in a therapeutically effective amount from about 1.0 mg/kg to about 15 mg/kg.

Also more preferably, said antigen binding molecule is used in a therapeutically effective amount from about 1.5 mg/kg to about 12 mg/kg.

Also more preferably, said antigen binding molecule is used in a therapeutically effective amount from about 1.5 mg/kg to about 4.5 mg/kg.

Also more preferably, said antigen binding molecule is used in a therapeutically effective amount from about 4.5 mg/kg to about 12 mg/kg.

Most preferably, said antigen binding molecule is used in a therapeutically effective amount of about 1.5 mg/kg.

Also most preferably, said antigen binding molecule is used in a therapeutically effective amount of about 4.5 mg/kg.

Also most preferably, said antigen binding molecule is used in a therapeutically effective amount of about 12 mg/kg.

The ABM compositions of the invention can be administered using conventional modes of administration including, but not limited to, intravenous, intraperitoneal, oral, intralymphatic or administration directly into the tumor. Intravenous administration is preferred.

In one aspect of the invention, therapeutic formulations containing the ABMs of the invention are prepared for storage by mixing an antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The ABMs of the present invention may be administered to a subject to treat a disease or disorder characterized by abnormal target antigen activity, such as a tumor, either alone or in combination therapy with, for example, a chemotherapeutic agent and/or radiation therapy. Suitable chemotherapeutic agents include cisplatin, doxorubicin, topotecan, paclitaxel, vinblastine, carboplatin, and etoposide.

Furthermore, the ABMs of the present invention can be used as a substitute for IVIG therapy. Although first introduced for the treatment of hypogammaglobulinemia, IVIG has since been shown to have broad therapeutic applications in the treatment of infectious and inflammatory diseases. Dwyer, J. M., New England J. Med. 326:107 (1992). The polyclonal specificities found in these preparations have been demonstrated to be responsible for some of the biological effects of IVIG. For example, IVIG has been used as prophylaxis against infectious agents and in the treatment of necrotizing dermatitis. Viard, I. et al., Science 282:490 (1998). Independent of these antigen specific effects, IVIG has well-recognized anti-inflammatory activities, generally attributed to the immunoglobulin G (IgG) Fc domains. These activities, first applied for the treatment of immune thrombocytopenia (ITP) (Imbach, P. et al., Lancet 1228 (1981); Blanchette, V. et al., Lancet 344:703 (1994)) have been extended to the treatment of a variety of immune mediated inflammatory disorders including autoimmune cytopenias, Guillain-Barre syndrome, myasthenia gravis, anti-Factor VIII autoimmune disease, dermatomyositis, vasculitis, and uveitis. (van der Meche, F. G. et al., New Engl. J. Med. 326:1123 (1992); Gajdos, P. et al., Lancet 406 (1984); Sultan, Y. et al., Lancet 765 (1984); Dalakas, M. C. et al., N.ew Engl. J. Med. 329:1993 (1993); Jayne, R. et al., Lancet 337:1137 (1991); LeHoang, P. et al., Ocul. Immunol. Inflamm. 8:49 (2000)). A variety of explanations have been put forward to account for these activities, including Fc receptor blockade, attenuation of complement-mediated tissue damage, neutralization of autoantibodies by antibodies to idiotype, neutralization of superantigens, modulation of cytokine production, and down-regulation of B cell responses. (Ballow, M., J. Allergy Clin. Immunol. 100:151 (1997); Debre, M. et al., Lancet 342:945 (1993); Soubrane, C. et al., Blood 81:15 (1993); Clarkson, S. B. et al., N. Engl. J. Med. 314:1236 (1986).

Lyophilized formulations adapted for subcutaneous administration are described in WO97/04801. Such lyophilized formulations may be reconstituted with a suitable diluent to a high protein concentration and the reconstituted formulation may be administered subcutaneously to the mammal to be treated herein.

The formulation herein may also contain more than one active compound as necessary for the particular indication being treated, preferably those with complementary activities that do not adversely affect each other. For example, it may be desirable to further provide a cytotoxic agent, chemotherapeutic agent, cytokine or immunosuppressive agent (e.g. one which acts on T cells, such as cyclosporin or an antibody that binds T cells, e.g., one which binds LFA-1). The effective amount of such other agents depends on the amount of antagonist present in the formulation, the type of disease or disorder or treatment, and other factors discussed above. These are generally used in the same dosages and with administration routes as used hereinbefore or about from 1 to 99% of the heretofore employed dosages.

The active ingredients may also be entrapped in microcapsules prepared, for examples, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antagonist, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

The compositions of the invention may be in a variety of dosage forms which include, but are not limited to, liquid solutions or suspension, tablets, pills, powders, suppositories, polymeric microcapsules or microvesicles, liposomes, and injectable or infusible solutions. The preferred form depends upon the mode of administration and the therapeutic application.

The compositions of the invention also preferably include conventional pharmaceutically acceptable carriers and adjuvants known in the art such as human serum albumin, ion exchangers, alumina, lecithin, buffer substances such as phosphates, glycine, sorbic acid, potassium sorbate, and salts or electrolytes such as protamine sulfate.

The most effective mode of administration and dosage regimen for the pharmaceutical compositions of this invention depends upon the severity and course of the disease, the patient's health and response to treatment and the judgment of the treating physician. Accordingly, the dosages of the compositions should be titrated to the individual patient. Nevertheless, an effective dose of the compositions of this invention will generally be in the range of from about 0:01 to about 2000 mg/kg.

The antigen binding molecules described herein may be in a variety of dosage forms which include, but are not limited to, liquid solutions or suspensions, tablets, pills, powders, suppositories, polymeric microcapsules or microvesicles, liposomes, and injectable or infusible solutions. The preferred form depends upon the mode of administration and the therapeutic application.

The composition comprising an ABM of the present invention will be formulated, dosed, and administered in a fashion consistent with good medical practice. Factors for consideration in this context include the particular disease or disorder being treated, the particular mammal being treated, the clinic condition of the individual patient, the cause of the disease or disorder, the site of delivery of the agent, the method of administration, the scheduling of administration, and other factors known to medical practitioners. The therapeutically effective amount of the antagonist to be administered will be governed by such considerations.

As a general proposition, the therapeutically effective amount of the antibody administered parenterally per dose will be in the range of about 0.1 to 20 mg/kg of patient body weight per day, with the typical initial range of antagonist used being in the range of about 2 to 10 mg/kg.

In a preferred embodiment, the ABM is an antibody, preferably a humanized antibody. Suitable dosages for such an unconjugated antibody are, for example, in the range from about 20 mg/m² to about 1000 mg/m². For example, one may administer to the patient one or more doses of substantially less than 375 mg/m² of the antibody, e.g., where the dose is in the range from about 20 mg/m² to about 250 mg/m², for example from about 50 mg/m² to about 200 mg/m².

Moreover, one may administer one or more initial dose(s) of the antibody followed by one or more subsequent dose(s), wherein the mg/m² dose of the antibody in the subsequent dose(s) exceeds the mg/m² dose of the antibody in the initial dose(s). For example, the initial dose may be in the range from about 20 mg/m² to about 250 mg/m² (e.g., from about 50 mg/m² to about 200 mg/m²) and the subsequent dose may be in the range from about 250 mg/m² to about 1000 mg/m².

As noted above, however, these suggested amounts of ABM are subject to a great deal of therapeutic discretion. The key factor in selecting an appropriate dose and scheduling is the result obtained, as indicated above. For example, relatively higher doses may be needed initially for the treatment of ongoing and acute diseases. To obtain the most efficacious results, depending on the disease or disorder, the antagonist is administered as close to the first sign, diagnosis, appearance, or occurrence of the disease or disorder as possible or during remissions of the disease or disorder.

In the case of ABMs of the invention used to treat tumors, optimum therapeutic results are generally achieved with a dose that is sufficient to completely saturate the antigen of interest on the target cells. The dose necessary to achieve saturation will depend on the number of antigen molecules expressed per tumor cell (which can vary significantly between different tumor types). Serum concentrations as low as 30 nM may be effective in treating some tumors, while concentrations above 100 nM may be necessary to achieve optimum therapeutic effect with other tumors. The dose necessary to achieve saturation for a given tumor can be readily determined in vitro by radioimmunoassay or immunoprecipitation.

In general, for combination therapy with radiation, one suitable therapeutic regimen involves eight weekly infusions of an ABM of the invention at a loading dose of 100-500 mg/m² followed by maintenance doses at 100-250 mg/m² and radiation in the amount of 70.0 Gy at a dose of 2.0 Gy daily. For combination therapy with chemotherapy, one suitable therapeutic regimen involves administering an ABM of the invention as loading/maintenance doses weekly of 100/100 mg/m², 400/250 mg/m², or 500/250 mg/m² in combination with cisplatin at a dose of 100 mg/m² every three weeks. Alternatively, gemcitabine or irinotecan can be used in place of cisplatin.

The ABM of the present invention is administered by any suitable means, including parenteral, subcutaneous, intraperitoneal, intrapulmonary, and intranasal, and, if desired for local immunosuppressive treatment, intralesional administration. Parenteral infusions include intramuscular, intravenous, intraarterial, intraperitoneal, or subcutaneous administration. In addition, the antagonist may suitably be administered by pulse infusion, e.g., with declining doses of the antagonist. Preferably the dosing is given by injections, most preferably intravenous or subcutaneous injections, depending in part on whether the administration is brief or chronic.

One may administer other compounds, such as cytotoxic agents, chemotherapeutic agents, immunosuppressive agents and/or cytokines with the antagonists herein. The combined administration includes coadministration, using separate formulations or a single pharmaceutical formulation, and consecutive administration in either order, wherein preferably there is a time period while both (or all) active agents simultaneously exert their biological activities.

It would be clear that the dose of the composition of the invention required to achieve cures may be further reduced with schedule optimization.

In accordance with the practice of the invention, the pharmaceutical carrier may be a lipid carrier. The lipid carrier may be a phospholipid. Further, the lipid carrier may be a fatty acid. Also, the lipid carrier may be a detergent. As used herein, a detergent is any substance that alters the surface tension of a liquid, generally lowering it.

In one example of the invention, the detergent may be a nonionic detergent. Examples of nonionic detergents include, but are not limited to, polysorbate 80 (also known as Tween 80 or (polyoxyethylenesorbitan monooleate), Brij, and Triton (for example Triton WR 1339 and Triton A 20).

Alternatively, the detergent may be an ionic detergent. An example of an ionic detergent includes, but is not limited to, alkyltrimethylammonium bromide.

Additionally, in accordance with the invention, the lipid carrier may be a liposome. As used in this application, a “liposome” is any membrane bound vesicle which contains any molecules of the invention or combinations thereof.

Articles of Manufacture

In another embodiment of the invention, an article of manufacture containing materials useful for the treatment of the disorders described above is provided. The article of manufacture comprises a container and a label or package insert on or associated with the container. Suitable containers include, for example, bottles, vials, syringes, etc. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). At least one active-agent in the composition is an ABM of the invention. The label or package insert indicates that the composition is used for treating the condition of choice, such as a non-malignant disease or disorder, for example a benign hyperproliferative disease or disorder. Moreover, the article of manufacture may comprise (a) a first container with a composition contained therein, wherein the composition comprises a first ABM which binds a target antigen and inhibits growth of cells which overexpress that antigen; and (b) a second container with a composition contained therein, wherein the composition comprises a second antibody which binds the antigen and blocks ligand activation of an antigen receptor. The article of manufacture in this embodiment of the invention may further comprises a package insert indicating that the first and second antibody compositions can be used to treat a non-malignant disease or disorder from the list of such diseases or disorders in the definition section above. Moreover, the package insert may instruct the user of the composition (comprising an antibody which binds a target antigen and blocks ligand activation of a target antigen receptor) to combine therapy with the antibody and any of the adjunct therapies described in the preceding section (e.g. a chemotherapeutic agent, an antigen-targeted drug, an anti-angiogenic agent, an immunosuppressive agent, tyrosine kinase inhibitor, an anti-hormonal compound, a cardioprotectant and/or a cytokine). Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The examples below explain the invention in more detail. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become apparent to those skilled in the art from the foregoing description and accompanying drawings. Such modifications are intended to fall within the scope of the appended claims.

All patents, applications, and publications cited in this application are hereby incorporated by reference in their entirety.

### EXAMPLES

Unless otherwise specified, references to the numbering of specific amino acid residue positions in the following Examples are according to the Kabat numbering system. Except where otherwise noted, the materials and methods used to make the antigen binding molecules in these working examples are in accordance with those set forth in the examples of U.S. patent application Ser. No. 10/981,738, which is hereby incorporated by reference in its entirety.

### Example 1

### Materials and Methods

Cell Lines, Expression Vectors and Antibodies

HEK293-EBNA cells were a kind gift of Rene Fischer (ETH Zürich). Additional cell lines used in this study were Jurkat cells (human lymphoblastic T cell, ATCC number TIB-152) or FcγRIIIa[Val-158]—as well as FcγRIIIa[Val-158/Gln-162]—expressing Jurkat cell lines, created as previously described (Ferrara, C. et al., Biotechnol. Bioeng. 93(5):851-861 (2006)). The cells were cultivated according to the instructions of the supplier. The DNAs encoding the shFcγRIIIa[Val-158] and shFcγRIIIa[Phe-158] were generated by PCR (Ferrara, C. et al., J. Biol. Chem. 281(8):5032-5036 (2006)) and fused to a hexahistidine tag resulting in the mature protein ending after residue 191 (NH₂-MRTEDL . . . GYQG(H₆)—COOH, numbering is based on the mature protein) as described (Shields, R. L. et al., J. Biol. Chem. 276(9):6591-6604 (2001)). The Asn-162 of shFcγRIIIa[Val-158] was exchanged for Gln by PCR. All expression vectors contained the replication origin OriP from the Epstein Barr virus for expression in HEK293-EBNA cells. GE and native anti-CD20 antibodies were produced in HEK-293 EBNA cells and characterized by standard methods. Neutral oligosaccharide profiles for the antibodies were analysed by mass spectrometry (Autoflex, Bruker Daltonics GmbH, Faellanden/Switzerland) in positive ion mode (Papac, D. I. et al., Glycobiol. 8(5):445-454 (1998)).

Production and Purification of Recombinant shFcγRIIIa Receptors

The shFcγRIIIa variants were produced by transient expression in HEK-293-EBNA cells (Jordan, M. et al., Nucl. Acids. Res. 24:596-601 (1996)) and purified by taking advantage of the hexahistidine tag using a HiTrap Chelating HP (Amersham Biosciences, Otelfingen/Switzerland) and a size exclusion chromatography step with HSP-EB buffer (0.01 M HEPES pH 7.4, 0.15 M NaCl, 3 mM EDTA, 0.005% Tween20). Human sFcγRIIb and mouse (m) sFcγRIIb were produced and purified as described (Sondermann, P. & Jacob., U., Biol. Chem. 380(6):717-721 (1999)). The concentration of all used proteins was determined as described (Gill, S. C. & von Hippel, P. H., Anal. Biochem. 182(2):319-326 (1989)).

Surface Plasmon Resonance (SPR)

SPR experiments were performed on a Biacore3000 with HBS-EP as running buffer (Biacore, Freiburg/Germany). Direct coupling of around 1,000 resonance units (RU) of human IgG glycovariants was performed on a CM5 chip using the standard amine coupling kit (Biacore, Freiburg/Germany). Different concentrations of soluble FcγRs were passed with a flowrate of 10 μl/min through the flow cells. Bulk refractive index differences were corrected for by subtracting the response obtained on flowing over a BSA-coupled surface. The steady state response was used to derive the dissociation constant K_{D} by non-linear curve fitting of the Langmuir binding isotherm. Kinetic constants were derived using the BIAevaluation program curve-fitting facility (v3.0, Biacore, Freiburg/Germany), to fit rate equations for 1:1 Langmuir binding by numerical integration.

Binding of IgG to FcγRIIIa-Expressing Cells

Experiments were conducted as previously described (Ferrara, C. et al., Biotechnol. Bioeng. 93(5):851-861 (2006)) Briefly, hFcγRIIIa-expressing Jurkat cells were incubated with IgG variants in PBS, 0.1% BSA. After one or two washes with PBS, 0.1% BSA, antibody binding was detected by incubating with 1:200 FITC-conjugated F(ab′)₂ goat anti-human, F(ab′)₂ specific IgG (Jackson ImmunoResearch, West Grove, Pa./USA) (Shields, R. L. et al., J. Biol. Chem. 276(9):6591-6604 (2001)). The fluorescence intensity referring to the bound antibody variants was determined on a FACS Calibur (BD Biosciences, Allschwil/Switzerland).

Modeling

Modeling was performed on the basis of the crystal structure of FcγRIII in complex with the Fc fragment derived from native IgG (PDB code 1e4k). For this purpose the coordinates of the carbohydrate moiety attached at Asn-297 of the Fc were duplicated and one of the glycans adjusted manually as rigid body to Asn-162 of FcγRIII with the pentasaccharide core directing to the position where the FUC residue of the Fc Asn-297 oligosaccharide is present. The model was not energy minimized and only created to visualize the proposed binding mode.

### Results

### Biochemical Characterization of Soluble FcγRIIIa Receptors and Antibody Glycovariants

ShFcγRIIIa[Val-158], shFcγRIIIa[Phe-158] and shFcγRIIIa[Val-158/Gln-162] were expressed in HEK293-EBNA cells and purified to homogeneity. The purified shFcγRIIIa-[Val-158] and -[Phe-158] migrate as a broad band when subjected to reducing SDS-PAGE with an apparent molecular weight of 40-50 kDa, which is slightly lower for the mutant shFcγRIIa[Val-158/Gln-162] (data not shown). This can be explained by the elimination of the carbohydrates linked to Asn-162. Upon enzymatic N-deglycosylation all three receptor variants migrate identically in the apparent molecular weight range of 25 to 30 kDa, and feature three bands as previously observed for membrane bound hFcγRIII (Edberg, J. C. & Kimberly, R. P., J. Immunol. 158(8):3849-3857 (1997), Ravetch, J. V. & Perussia, B., J. Exp. Med. 170(2):481-497 (1989)). This heterogeneous pattern may result from the presence of O-linked carbohydrates.

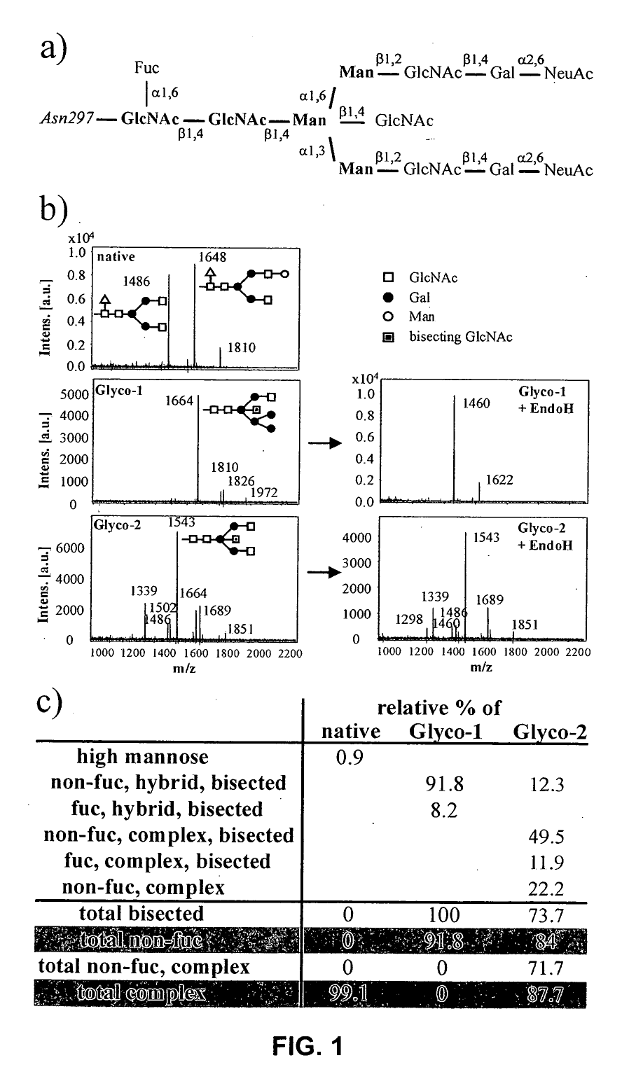

The native antibody glycosylation pattern is characterized by biantennary, fucosylated complex oligosaccharides (FIG. 1b, c), heterogeneous with respect to terminal galactose content. GE antibodies were produced in a cell line overexpressing N-acetylglucosaminyltransferase III (GnT-III), an enzyme catalysing the addition of a bisecting GlcNAc (FIG. 1a) to the β-mannose of the core. Two different GE antibody variants were generated, Glyco-1 was produced by overexpression of GnT-III alone and Glyco-2 by co-expression of GnT-III and recombinant Man-II (Ferrara, C. et al., Biotechnol. Bioeng. 93(5):851-861 (2006), FIG. 1b). Both Glyco-1 and Glyco-2 are characterized by high proportions of bisected, non-fucosylated oligosaccharides (88% hybrid type and 90% complex type, respectively, FIG. 1c). We have previously shown that both forms give similar increases in affinity for FcγRIIIa and increased ADCC relative to native antibodies but a differ in their reactivity in CDC assays (Ferrara, C. et al., Biotechnol. Bioeng. 93(5):851-861 (2006)). IgG-oligosaccharide modifications lead to antibodies with increased affinity for shFcγRIIIa

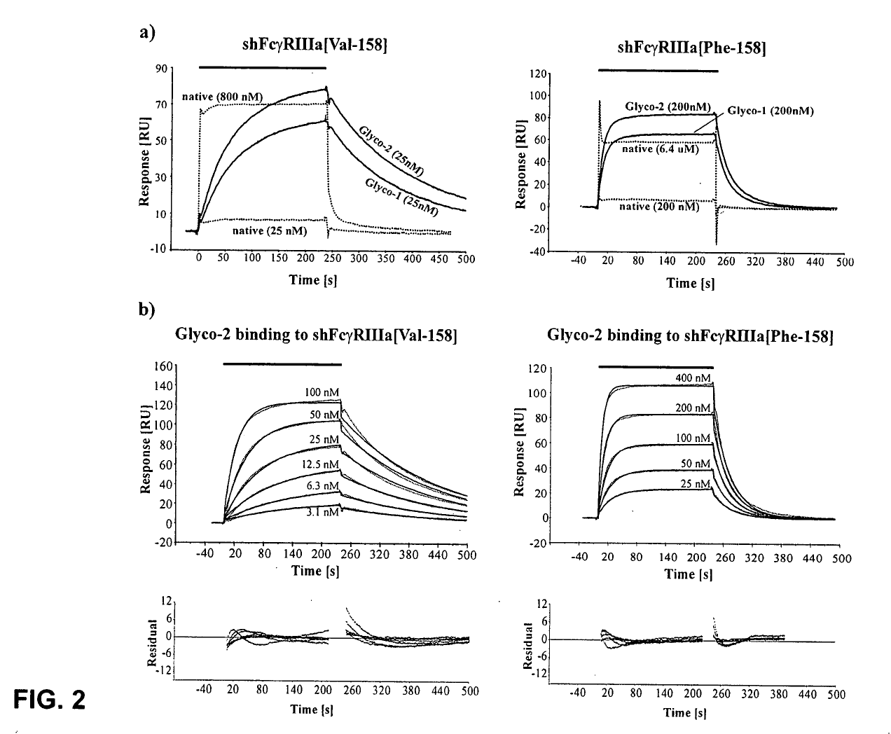

The interactions of antibody glycovariants with shFcγRIIIa variants ([Val-158], [Phe-158] and [Val-158/Gln-162]), shFcγRIIb and smFcγRIIb were analysed by SPR. Binding of shFcγRIIIa[Val-158] to the GE antibodies was up to 50 fold stronger than to the native antibody (K_{D(Glyco-2)} 0.015 μM v's K_{D(native)} 0.75 μM, Table 6). Importantly, the “low affinity” polymorphic form of the receptor, shFcγRIIIa[Phe-158] also bound to the GE antibodies with significantly higher affinity than to the native antibody (K_{D(Glyco-1)} 0.27 μM (18 fold), K_{D(Glyco-2)} 0.18 μM (27 fold), K_{D(native)} 5 μM (Table 6)). Dissociation of both receptor variants from native IgG was too fast to enable a direct determination of kinetic constants for these interactions. Although it was not possible to obtain kinetic parameters for binding of the receptors to native Ab, overlaying the experimental data clearly shows that a major effect of glycoengineering the antibodies is decreased dissociation of the receptors (FIG. 2a). To estimate dissociation rates from native IgG the experimental data was overlayed with curves simulating different dissociation rate constants (not shown). This indicated that the entire increase in affinity upon glyco-engineering could be accounted for by decreased k_{off}. The association rate constants (kₒₙ) of the two polymorphic forms of shFcγRIIIa for GE antibodies were similar but the dissociation rate of sFcγRIIIa[Phe-158] was significantly faster and largely accounts for the lower affinity of this receptor (Table 6).

The affinity of the antibodies towards human and murine FcγRIIb was also measured Both GE and native IgGs bound the human inhibitory receptor shFcγRIIb with similar affinities in the range of K_{D}=1.55-2.40 μM (Table 6). For the murine version of this receptor, the affinity towards human IgG1 was also unaltered by glyco-engineering but surprisingly was 3.4- to 5.5-times that of the human FcγRIIb receptor (Table 6). The dissociation constant (K_{D}) for the interaction of the native antibody with sh/mFcγRIIb could only be determined by steady state analysis (Table 6) because the equilibrium was reached too fast for a kinetic evaluation (FIG. 2a).

TABLE 6
Summary of affinity constants determined by equilibrium and kinetic analysis

| IgG1 | Fcγ receptor | kₒₙ (×10⁵ M⁻¹s⁻¹) | k_{off} (×10⁻³ s⁻¹) | K_{D}-kinetic (μM) | K_{D}-steady state (μM) |
|---|---|---|---|---|---|
| native | shFcγRIIIa[Val-158] | nd* | nd* | nd* | 0.75 ± 0.04 |
| Glyco-1 | shFcγRIIIa[Val-158] | 2.4 ± 0.01 | 5.8 ± 0.01 | 0.024 ± <0.001 | nd |
| Glyco-2 | shFcγRIIIa[Val-158] | 3.2 ± 0.01 | 5.1 ± 0.01 | 0.016 ± <0.001 | 0.015 |
| native | shFcγRIIIa[Phe-158] | nd* | nd* | nd* | 5 ± 0.3 |
| Glyco-1 | shFcγRIIIa[Phe-158] | 1.6 ± 0.09 | 32 ± 0.1 | 0.20 ± 0.001 | 0.27 ± 0.01 |
| Glyco-2 | shFcγRIIIa[Phe-158] | 2.3 ± 0.01 | 29 ± 0.1 | 0.13 ± 0.001 | 0.18 ± 0.01 |
| native | shFcγRIIIa[Val-158/Gln-162] | 5.9 ± 0.05 | 90 ± 0.4 | 0.16 ± 0.001 | 0.24 ± 0.01 |
| Glyco-1 | shFcγRIIIa[Val-158/Gln-162] | 4.7 ± 0.02 | 89 ± 0.5 | 0.19 ± 0.001 | 0.30 ± 0.01 |
| Glyco-2 | shFcγRIIIa[Val-158/Gln-162] | 8.1 ± 0.06 | 72 ± 0.3 | 0.09 ± 0.001 | 0.20 ± 0.01 |
| native | shFcγRIIb | nd* | nd* | nd* | 2.4 ± 0.11 |
| Glyco-1 | shFcγRIIb | nd* | nd* | nd* | 2.4 ± 0.05 |
| Glyco-2 | shFcγRIIb | nd* | nd* | nd* | 1.6 ± 0.05 |
| native | smFcγRIIb | nd* | nd* | nd* | 0.44 ± 0.01 |
| Glyco-1 | smFcγRIIb | nd* | nd* | nd* | 0.69 ± 0.01 |
| Glyco-2 | smFcγRIIb | nd* | nd* | nd* | 0.46 ± 0.01 |

Errors are calculated for the curve fitting and for the deviation of two experiments (more detail is needed, I anm not sure how this was done)
nd = not determined
*kinetic too fast for exact determination
h, human and
m, mouse

FcγRIIIa-Glycosylation Regulates Binding to Antibody Glycovariants

A mutant form of hFcγRIIIa that is not glycosylated at Asn162 (shFcγRIIIa[Val-158/Gln-162]) was used to analyze the influence of a potential carbohydrate-mediated interaction between oligosaccharide at this position in the receptor and IgG. Interestingly, upon removal of Asn162, native IgG showed a threefold increase (K_{D}=0.24 μM c.f. 0.75 μM) in affinity for the receptor, whereas GE antibodies showed an over 13-fold decrease in affinity (Table 6). For binding to GE antibodies, removal of the receptor glycosylation site resulted in an almost twofold increase in kₒₙ, but an over 14-fold increase in k_{off} (Table 6). Steady state and kinetically determined K_{D}s differed by 1.6 to 2.2 fold for binding of shFcγRIIIa[Val-158/Gln-162]. This discrepency most likely results from a high error in fitting the very fast dissociation observed.

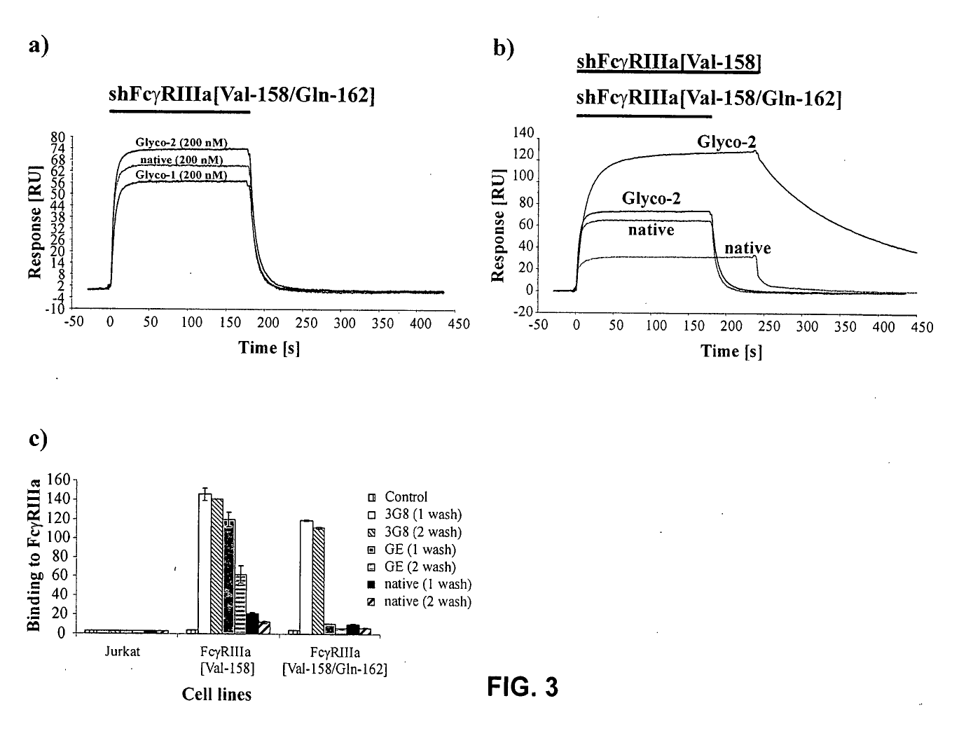

The SPR-based results were corroborated in a cellular system using Jurkat cells expressing FcγRIIIa. Jurkat cells (human T cell line) represent a natural environment for FcγRIIIa expression (Edberg, J. C. & Kimberly, R. P., J. Immunol. 158(8):3849-3857 (1997)). The anti-FcγRIII mAb 3G8, which does not discriminate between FcγRIIIa[Val-158] and FcγRIIIa[Val-158/Gln-162] (Drescher, B. et al., Immunology 110(3):335-340 (2003)), was used to monitor FcγRIII expression in these cell lines. In this experiment GE antibodies bound FcγRIIIa[Val-158] better than the native antibody (FIG. 3c). Binding to FcγRIIIa[Val-158/Gln-162] was however almost undetectable for all IgG variants, including native IgG (FIG. 3c). The very fast dissociation rate constants found in the SPR experiment for binding of FcγRIIIa[Val-158/Gln-162] to all three IgG variants could explain this negligible binding in the cellular assay.

### Discussion

Kinetic Analysis of the FcγRIIIa/IgG Interaction

Overall our measured K_{D}s agree with those previously published by Okazaki et al. (Okazaki, A. et al., J. Mol. Biol. 336(5):1239-1249 (2004)). These authors concluded that the affinity increase of the non-fucosylated (GE) antibody is predominantly caused by an increase in kₒₙ. In contrast, although we could not quantify kₒₙ and k_{off} for binding to native IgG due to the high velocity of the reaction, a qualitative analysis of these binding events compared with those involving GE antibodies, clearly shows significantly faster dissociation of the receptor variants from native IgG (FIG. 2a). It can therefore be concluded that either new interactions between the binding partners are formed or the present ones are improved.

The glycosylation of FcγRIIIa at Asn162 Modulates Binding to Antibodies

FcγRIIIa of mammalian origin is a highly glycosylated protein with five N-linked glycosylation sites. As hypothesised from the crystal structure of the FcγRIII/IgG1-Fc complex (Sondermann, P. et al., Nature 406:267-273 (2000) (hereby incorporated by reference in its entirety), elimination of glycosylation at Asn162 results in an enhanced affinity for native IgG1 (Drescher, B. et al., Immunology 110(3):335-340 (2003)) probably by the elimination of a steric clash of the hFcγRIIIa[Asn162] carbohydrate moiety with the Fc. Removal of carbohydrate at the other four N-glycosylation sites does not effect affinity for native IgG (Drescher, B. et al., Immunology 110(3):335-340 (2003)).

A mutant version of the high affinity receptor which is unglycosylated at position 162 (shFcγRIIIa[Val-158/Gln-162]) was constructed to further investigate the importance of glycosylation of IgG and FcγRIIIa to their interaction. As expected, we found an increase in affinity for the interaction between native antibody and the Asn162-glycosylation deficient FcγRIIIa[Val-158/Gln-162] (3-fold, Table 6). However GE antibodies bound more than ten times weaker to the mutant receptor than to native, glycosylated receptor shFcγRIIIa[Val-158] (Table 6) indicating that the oligosaccharide attached to Asn162 of hFcγRIIIa favors the interaction between IgG1 and this Fc receptor. This data was corroborated in a cellular assay system, where GE antibodies bound significantly better to FcγRIIIa[Val-158]-expressing cells than to FcγRIIIa[Val-158/Gln-162]-expressing cells (FIG. 3c). In another set of experiments the present inventors demonstrated that the binding behaviour of antibodies with a considerably reduced fucose content and lacking the bisecting GlcNAc (Fuc-) generated by the expression in YO myeloma cells (Lifely, M. R. et al., Glycobiol. 5(8):813-822 (1995)) is very similar to that of the GE antibodies (i.e. affinity for the deglycosylated receptor is lower than for the native receptor), The absence of the fucose residue in the GE and Fuc-antibodies therefore appears to be mainly responsible for the enhanced affinity of the glycosylated form of the receptor for these antibodies (See, e.g., Shinkawa, T. et al., J. Biol. Chem. 278(5):3466-73 (2003); Shields, R. L. et al., J. Biol. Chem. 277(30):26733-26740 (2002)).

In summary, the improved interaction of GE antibodies with FcγRIIa is modulated by the carbohydrate moieties of both binding partners. From crystal structures of IgG-Fc fragments, it is known that the interaction of carbohydrates with the protein is mainly stabilized by hydrophobic, preferably aromatic residues (Huber, R. et al., Nature 264(5585):415-420 (1976)). Particularly relevant to our results is the intense contact between the Fc's Tyr296 and the Fc's fucose. GE antibodies do not contain this fucose and we hypothesise that upon complex formation with FcγRIIIa the receptor carbohydrate attached at Asn162 forms close, favorable contacts with GE Fc, thereby accounting for the high affinity of this interaction.

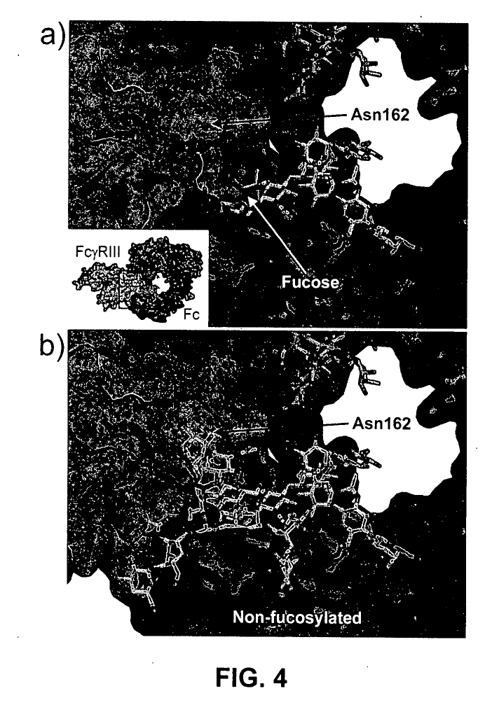

A model of the proposed interaction demonstrates that three mannose residues of the pentasaccharide core of the oligosaccharide linked to Asn162 of FcγRIIIa could reach the IgG Fc Tyr296 (FIG. 4). Such a binding mode would favor the interaction of the FcγRIIIa carbohydrate with the Fc's Tyr-296, which is also accompanied by a much closer contact of the carbohydrate to the protein moiety of the IgG. This model can be used to identify amino acid substitutions on the Fc surface which further strengthen the contact with the FcγRIII carbohydrate. In a recent study, Okazaki et al. suggested that non-fucosylated antibodies bind FcγRIIIa with increased affinity as a result of a newly formed bond between Tyr-296 of the Fc and Lys-128 of the FcγRIIIa (Huber, R. et al., Nature 264(5585):415-420 (1976)). However, it has now been found that the increased affinity of non-fucosylated antibodies depends on glycosylation of the receptor. Such an effect of receptor glycosylation indicates that a Fc-Tyr296/Lys128-FcγRIIIa bond is insignificant to the affinity between GE antibodies and FcγRIIIa.

FcγRIII a and b forms are the only forms of the human FcγR that possess N-glycosylation sites within the binding region to IgG. We therefore conclude that affinity for IgG will be influenced by receptor glycosylation only for these two FcγRs. Comparison of the amino acid sequences of FcγRIII from other species indicates that the N-glycosylation site Asn162 is shared by FcγRIII from macaca, cat, cow and pig, whereas it is lacking in the known rat and mouse FcγRIII. Recently mouse and rat genes were identified (CD16-2 and protein data bank number NP_{—}997486, respectively) with high homology to the human FcγRIII and which encode proteins containing the Asn162 glycosylation site were identified (Huber, R. et al., Nature 264(5585):415-420 (1976)), but functional expression of the proteins has yet to be demonstrated.

The presence of an Asn162-FcγRIIIa glycosylation site likely enables the immune system to tune the affinity towards FcγRIII either by differential FcγRIII glycosylation (Edberg, J. C. & Kimberly, R. P., J. Immunol. 158(8):3849-3857 (1997)) or by modulation of the fucose content of IgG.

The Immunological Balance Between Activating and Inhibitory FcγRs

It has been proposed that an improvement of the ratio between activating and inhibitory signals will enhance the efficacy of therapeutic antibodies (Clynes, R. A. et al., Nat. Med. 6(4):443-446 (2000); Stefanescu, R. N. et al., J. Clin. Immunol. 24(4):315-326 (July 2004)). In the current study the inhibitory shFcγRIIb receptor was found to have a similar affinity for native and GE antibodies, whereas both activating receptor variants bound with higher affinity to the GE antibodies than to the native antibody (Table 6). This indicates that the oligosaccharide modifications of GE antibodies exclusively increase the affinity for the activating receptors and indicates that these GE antibodies will show enhanced therapeutic efficacy.

The inhibitory receptors sFcγRIIbs from mouse and human are not glycosylated at Asn162. The lack of discrimination for GE antibodies displayed by both these receptors is consistent with glycosylation of FcγRs at Asn62 being essential for increased binding to non-fucosylated IgGs.

The finding that murine FcγRII has significantly higher affinity than human FcγRIIb for the antibodies may be important for the correct interpretation of in vivo experiments using mouse models. Enhanced binding to the inhibitory receptor in a mouse model may result in a different threshold of the immune response than that in humans.

### Conclusion

These studies demonstrate the importance of the carbohydrate moieties of both FcγRIIIa and IgG for their interaction. The data provides further insight into the complex formation and identifies the important distinct interaction between the glycans of FcγRIIIa and the Fc of non-fucosylated IgG glycoforms on the molecular level. This finding offers the basis for the design of new antibody variants that make further productive interactions with the carbohydrate of FcγRIIIa, which has important implications for therapies with monoclonal antibodies.

### Example 2

Generation of Antibody Mutants

Antibody mutants were generated using standard molecular biology methods (e.g. mutagenic PCR, see Dulau L, et al. Nucleic Acids Res. 11; 17(7):2873 (1989)), using a humanized IgG1 as template with a specificity for CD20 or EGFR. The resulting antibody mutant encoding DNA was subsequently cloned into an OriP containing plasmid and used for the transient transfection of HEK293-EBNA cells (Invitrogen, Switzerland) as previously described (Jordan, M., et al., Nucleic Acids Res. 24, 596-601 (1996)). Glycoengineered antibodies were produced by co-transfection of the cells with two plasmids coding for antibody and chimeric GnT-III, at a ratio of 4:1, respectively, while for unmodified antibody the plasmids coding for the carbohydrate-modifying enzymes were omitted. The supernatant was harvested five days after transfection. For some of the experiments the antibody was purified from the supernatant using two sequential chromatographic steps as described (Umaña, P., et al., Nat. Biotechnol. 17, 176-180 (1999)), followed by size exclusion chromatography. The peak fractions containing the monomeric antibody were pooled and concentrated.

Quantitation of the Antibody in Culture Supernatant

Direct quantitation of the antibody present in the supernatant of the transfected EBNA cells was performed using Protein A chromatography. For that purpose 100 μl of the supernatant was applied to a column filled with Protein A immobilized to a resin. The bound antibody was eluted using a buffer of pH 3 after the removal of unbound proteins with a washing step. The absorbance at a wavelength of 280 nm caused by the eluting antibody was integrated and used for its quantitation in combination with antibody standards of known concentration.

Carbohydrate Analysis

HPLC fractions containing the antibody or purified antibodies were buffer exchanged to 2 mM TRIS pH 7.0 and concentrated to 20 μl. Oligosaccharides were enzymatically released from the antibodies by N-Glycosidase digestion (PNGaseF, EC 3.5.1.52, QA-Bio, San Mateo, Calif., USA) at 0.05 mU/μg protein in 2 mM Tris, pH 7 for 3 hours at 37° C. A fraction of the PNGaseF-treated sample was subsequently digested with Endoglycosidase H (EndoH, EC 3.2.1.96, Roche, Basel/Switzerland) at 0.8 mU/μg protein to distinguish between complex and hybrid carbohydrates and incubated for 3 hours at 37° C. The released oligosaccharides were adjusted to 150 mM acetic acid prior to purification through a cation exchange resin (AG50W-X8 resin, hydrogen form, 100-200 mesh, BioRad, Reinach/Switzerland) packed into a micro-bio-spin chromatography column (BioRad, Reinach/Switzerland) as described (Papac, D. I., Briggs, J. B., Chin, E. T., and Jones, A. J. (1998) Glycobiology 8, 445-454).

1 μl of sample was mixed in an Eppendorff tube with 1 μl of the freshly prepared matrix, which is prepared by dissolving 4 mg 2,5-dihydroxybenzoic acid and 0.2 mg 5-methoxysalicylic acid in 1 ml ethanol/10 mM aqueous sodium chloride 1:1 (v/v). Then, 1 μl of this mixture was transferred to the target plate. The samples were allowed to dry before measurement using an Autoflex MALDI/TOF (Bruker Daltonics, Faellanden/Switzerland) operating in positive ion mode.

FcγRIIIa Binding Assay

Jurkat (DSMZ-number ACC-282) or CHO cells (ECACC-number 94060607) were transfected with a plasmid encoding hFcγRIIIa in combination with the γ-chain and incubated with known concentrations of IgG mutants in PBS and 0.1% BSA for 30 min at 4° C. After several washes antibody binding was detected by incubation for 30 min at 4° C. with 1:200 FITC-conjugated F(ab′)₂ goat anti-human F(ab′)₂ specific IgG (Jackson ImmunoResearch, West Grove, Pa., USA). The fluorescence intensity of 10000 cells corresponding to the bound antibody variants was determined on a FACS Calibur (BD Biosciences, Allschwil, Switzerland).

In a similar manner a cell line was generated expressing hFcγRIIIa which is unglycosylated at position Asn162 by exchanging this residue for a glutamine (FcγRIIIa-Q162). The binding assay was performed as described above using this cell line.

Using these methods, IgG mutants can be identified that show an increased binding to hFcγRIIIa when non-fucosylated compared to the unmodified (fucosylated) mutant antibody. Furthermore, such identified IgG mutants have preferably an increased affinity to FcγRIIIa but not unglycosylated FcγRIIIa-Q162.

FcγRIIb Binding Assay

CHO cells (ECACCnumber 94060607) were transfected with a plasmid encoding hFcγRIIb leading to its surface expression. In case the tested antibody mutants are directed against EGFR, Raji cells can be used as well for this assay. The cells were incubated with known concentrations of IgG mutants in PBS and 0.1% BSA for 30 min at 4° C. After several washes antibody binding was detected by incubating for 30 min at 4° C. with 1:200 FITC-conjugated F(ab′)₂ goat anti-human F(ab′)₂ specific IgG (Jackson ImmunoResearch, West Grove, Pa., USA). The fluorescence intensity of 10000 cells corresponding to the bound antibody variants was determined on a FACS Calibur (BD Biosciences, Allschwil, Switzerland).

Using the methods described above, IgG mutants can be identified that show preferably an unaltered binding to hFcγRIIb compared to the unmodified antibody. In another preferred embodiment of this invention molecules that do preferably bind to FcγRIII compared to the inhibitory receptor FcγRIIb are claimed. This consequently also includes mutants that show an intermediate binding to FcγRIII (i.e. between the wildtype antibody and the glycoengineered antibody) but almost no binding to FcγRIIb. Such claimed antibody mutants have a “specificity ratio” above 1. By “specificity ratio” is meant specificity to human FcγRIII receptor as the ratio of binding affinity to another human Fcγ receptor.

ADCC Assay

EGFR positive A431 cells (ATCC-number CRL-1555) or CD20-positive Raji cells (ATCC-number CCL-86) were incubated with purified antibody mutants or culture supernatants containing them (Invitrogen AG, Basel, Switzerland) for 10 min serially diluted with AIM-V medium (Invitrogen, Switzerland). Freshly prepared peripheral blood mononuclear cells (PBMC) from a donor heterozygous for FcγRIIIa-Val/Phe158 and lacking FcγRIIc expression were added to the wells at an effector to target ratio of 25:1. Alternatively, NK-92 cells (DSMZ-number ACC-488) transfected with hFcγRIIIa and the γ-chain were used instead of PBMCs. After four hours of incubation at 37° C., 100 μl of the cell-free supernatant were transferred to a new plate for the detection of LDH released by the lysed cells using the Cytotoxicity Detection Kit (Roche, Basel, Switzerland) according to the protocol of the manufacturer.

Modelling

Modelling was performed on basis of the crystal structure of FcγRIII in complex with the Fc fragment derived from native IgG (PDB code 1e4k). For that purpose the coordinates of the carbohydrate moiety attached at Asn-297 of the Fc were duplicated and one of the glycans adjusted manually as rigid body to Asn-162 of FcγRIII with the pentasaccharide core directing to the position where the FUC residue is present. The model was not minimized and only created to visualize the proposed binding mode.

### Example 3

### Materials and Methods

Expression of Antibody Mutants in Hek293 EBNA Cells

The antibody mutants were generated by site-directed mutagenesis and the resulting DNA was cloned into an OriP containing plasmid and used for the transient transfection of HEK293-EBNA cells (Invitrogen, Switzerland) as previously described (Jordan, M., et al., Nucleic Acids Res. 24:596-601 (1996)). Several glycoforms of these antibodies were prepared by cotransfection of the antibody-encoding plasmid either with chimeric GnT-III (G1, characterized by mainly hybrid non-fucosylated bisected carbohydrates), or with chimeric GnT-III and ManII (G2, characterized by high proportions of complex non-fucosylated bisected carbohydrates). For unmodified antibody, the plasmids coding for the carbohydrate-modifying enzymes were omitted. The supernatants were harvested five days after transfection.

Quantitation and Purification of the Antibody in Culture Supernatant for Carbohydrate Analysis and Surface Plasmon Resonance.

Direct quantitation of the antibody present in the supernatant of the transfected EBNA cells was performed using Protein A chromatography. For that purpose, 100 μl of the supernatant was applied to a column filled with Protein A immobilized on a resin. The bound antibody was eluted using a buffer of pH 3 after the removal of unbound proteins with a washing step. The absorbance at a wavelength of 280 nm caused by the eluting antibody was integrated and used for its quantitation in combination with antibody standards of known concentration. The eluted sample was used for carbohydrate analysis.

For surface plasmon resonance application, 5 ml of supernatant were incubated end-over-end with 20 μl of ProteinA Sepharose beads (rmp Protein A Sepharose Fast Flow, Amersham Biosciences, Otelfingen, Switzerland) overnight at room temperature. The sample was transferred to an empty microspin column (BioRad, Reinach, Switzerland) and centrifuged at 1000×g for 1 min. The retained beads were washed once with 10 mM Tris, 50 mM glycine, 100 mM sodium chloride, pH 8.0. Elution was performed by incubation with 120 μl of 10 mM Tris, 50 mM glycine, 100 mM sodium chloride, pH 3.0 for 5 min followed by centrifugation at 1000×g for 2 min in an Eppendorf tube containing 6 μl 2 M Tris, pH8.0 for neutralization.

Carbohydrate Analysis

The purified antibodies were buffer exchanged to 2 mM TRIS pH 7.0 and concentrated to 20 μl. Oligosaccharides were enzymatically released from the antibodies by N-Glycosidase digestion (PNGaseF, EC 3.5.1.52, QA-Bio, San Mateo, Calif., USA) at 0.05 mU/μg protein in 2 mM Tris, pH 7 for 3 hours at 37° C. The released oligosaccharides were adjusted to 150 mM acetic acid prior to purification through a cation exchange resin (AG50W-X8 resin, hydrogen form, 100-200 mesh, BioRad, Reinach, Switzerland) packed into a micro-bio-spin chromatography column (BioRad, Reinach, Switzerland) as described (Papac, D. I., et al., Glycobiology 8, 445-454 (1998)).

1 μl of sample was mixed in an Eppendorff tube with 1 μl of the freshly prepared matrix, which is prepared by dissolving 4 mg 2,5-dihydroxybenzoic acid and 0.2 mg 5-methoxysalicylic acid in 1 ml ethanol/10 mM aqueous sodium chloride 1:1 (v/v). Then, 1 μl of this mixture was transferred to the target plate. The samples were allowed to dry before measurement using an Autoflex MALDI/TOF (Bruker Daltonics, Faellanden, Switzerland) operating in positive ion mode.

Size Exclusion Chromatography

For SPR studies the protein A-enriched sample (100 μl) was purified by size exclusion chromatography with an Agilent 1100 system with autosampler and MAD unit using a Tricorn Superdex 200 10/300 GL column (Amersham Biosciences, Otelfingen, Switzerland) and HSP-EB buffer (0.01 M HEPES pH 7.4, 0.15 M NaCl, 3 mM EDTA, 0.005% Tween20) as running buffer. The absorbance at a wavelength of 280 nm caused by the eluting antibody was integrated and used for its quantitation in combination with antibody standards of known concentration.

Expression of Soluble shFcγRIIIa-His₆ and shFcγRIIb-His₆

ShFcγRIIIa-His₆ and shFcγRIIb-His₆ were produced by transient expression in HEK293-EBNA cells (Jordan, M. et al., Nucl. Acids. Res. 24:596-601 (1996)) and purified to homogeneity by taking advantage of the hexahistidine tag using a HiTrap Chelating HP (Amersham Biosciences, Otelfingen, Switzerland) and a size exclusion chromatography step with HSP-EB buffer (0.01 M HEPES pH 7.4, 0.15 M NaCl, 3 mM EDTA, 0.005% Tween20). The concentration of the proteins was determined as described (Gill, S. C. & von Hippel, P. H., Anal. Biochem. 182(2):319-326 (1989)).

Surface Plasmon Resonance

SPR experiments were performed on a Biacore1000 with HBS-EP as running buffer (Biacore, Freiburg, Germany). Direct coupling of around 200-500 resonance units (RU) of human Fcγ Receptors was performed on a CM5 chip using the standard amine coupling kit (Biacore, Freiburg, Germany). A set of concentrations of IgG mutants were passed with a flowrate of 30 μl/min through the flow cells. Bulk refractive index differences were corrected for by subtracting the response obtained on flowing over a reference surface without protein immobilized. The steady state response was used to derive the dissociation constant K_{D} by non-linear curve fitting of the Langmuir binding isotherm. Kinetic constants were derived using the BIAevaluation program curve-fitting facility, to fit rate equations for 1:1 Langmuir binding by numerical integration.

Results

The antibodies were diluted in HBS-EP and passed over surfaces with immobilized receptors. Using the described method it is now possible to identify amino acid mutants that can not be identified when using a nonglycoengineered version. For example the antibody mutants S239W and F243E show a decreased affinity to FcγRIIIa when not glycoengineered (non-GE) but have almost an identical K_{D} compared to that of the control antibody when also glycoengineered (GE).

According to the described principle successful mutants should feature either one of the following characteristics:

A. The GE IgG-mutant has an increased affinity to FcγRIIIa compared to the GE IgG lacking the amino acid modification.

B. The GE IgG mutant has an increased affinity to FcγRIIIa, mediated by the carbohydrate moiety of FcγRIIIa. These mutants can be identified by binding to FcγRIIIa lacking glycosylation at position 162 (FcγRIIIa-Q162).

C. The mutants have either an increased kₒₙ or a reduced k_{off} compared to the GE control antibody.

According to the above-described characteristics, the following three groups have been defined:

TABLE 7
Table 7 - The three groups have been divided according to the affinities

| | |
|---|---|
| shFcγRIIIa | shFcγRIIIa-Q162 |

| Group | K_{D} non-GE | K_{D} GE | K_{D} non-GE | K_{D} GE |
|---|---|---|---|---|
| 1 | > | ≧ | > | ≧ |
| 2 | < | < | < | < |
| 3 | ≧ | ≦ | > | = |

(increased (>), decreased (<), or unchanged (=) K_{D}) for the IgG mutants (as non-GE or GE glycoforms) to shFcγRIIIa and shFcγRIIIa-Q162 compared to the control antibody in the respective glycoform.

The following IgG mutants were selected:

TABLE 8
Table 8 - Amino acid substitutions of the selected mutants

| Mutant | Substitution |
|---|---|
| 43 | S239D |
| 98 | S239E |
| 20 | S239W |
| 85 | F243H |
| 22 | F243E |
| 88 | T260H |
| 9 | H268D |
| 30 | H268E |

TABLE 9
Table 9 - Dissociation constants of the interactions between IgG mutants
and shFcγRIIIa or shFcγRIIIa-Q162. Interactions between immobilized
shFcγRIIIa-H6 and IgG mutants were determined by kinetic analysis while
interactions between immobilized shFcγRIIIa-Q162-H6 and
IgG mutants were determined by steady state analysis.

| | | | shFcγRIIIa-H6 | | | | |
|---|---|---|---|---|---|---|---|
| | | | kₒₙ 1E5 | k_{off} 1E-3 | K_{D} | shFcγRIIIa_Q162_H6 K_{D} | |
| n^{o} | Mutant | glycoform | (1/Ms) | (1/s) | (nM) | (nM) | Group |
| | control | non-GE | / | / | 245.60 | 214.40 | — |
| | | G2 | 6.77 | 11.43 | 16.89 | 168.80 | |
| | | G1 | 5.17 | 13.86 | 26.78 | 306.20 | |
| 9 | H268D | non-GE | / | / | 123.80 | 87.34 | 2 |
| | | G2 | 5.45 | 4.448 | 8.17 | 85.96 | |
| | | G1 | 3.50 | 7.255 | 20.74 | 169.50 | |
| 20 | S239W | non-GE | / | / | 688.20 | 372.00 | 1 |
| | | G2 | 1.12 | 2.863 | 25.54 | 371.40 | |
| | | G1 | 1.07 | 6.232 | 58.44 | nb | |
| 22 | F243E | non-GE | / | / | 886.90 | 428.30 | 1 |
| | | G2 | 1.22 | 2.868 | 23.52 | 340.10 | |
| | | G1 | 2.66 | 9.605 | 36.06 | nb | |
| 30 | H268E | non-GE | / | / | 188.80 | 152.50 | 2 |
| | | G2 | 5.75 | 6.25 | 10.87 | 138.80 | |
| | | G1 | 3.42 | 7.679 | 22.44 | 161.30 | |
| 43 | S239D | non-GE | / | / | 85.66 | 122.00 | 2 |
| | | G2 | 3.32 | 2.33 | 7.01 | 128.20 | |
| | | G1 | 2.19 | 2.456 | 11.23 | 121.30 | |
| 85 | F243H | non-GE | / | / | 542.1 | 382 | 1 |
| | | G2 | 5.01 | 8.689 | 17.33 | 168.50 | |
| 88 | T260H | non-GE | / | / | 276.50 | 289.60 | 3 |
| | | G2 | 15.12 | 19.54 | 12.93 | 160.40 | |
| 98 | S239E | non-GE | / | / | 155.30 | 169.80 | 2 |
| | | G2 | 3.57 | 2.79 | 7.83 | 107.40 | |

non-GE = nonglycoengineered;
G1 = glycoform prepared with GnT-III;
G2 = glycoform prepared with GnT-III and ManII.

TABLE 10
Table 10 - Comparison with control antibody of the interactions
obtained with selected IgG mutants. IgG mutants glycoforms were
compared to their respective glycoform of the original
antibody and were labeled as binding with increased (+),
unchanged (=) or reduced (−) K_{D} or k_{off}.

| | | | K_{D} | K_{D} | k_{off} |
|---|---|---|---|---|---|
| | | | RIIIa-H6 | RIIIa-Q162-H6 | RIIIa-H6 |
| 9 | H268D | non-GE | − | − | nd* |
| | | G2 | − | − | − |
| | | G1 | − | − | − |
| 20 | S239W | non-GE | + | + | nd* |
| | | G2 | + | + | − |
| | | G1 | + | + | − |
| 22 | F243W | non-GE | + | + | nd* |
| | | G2 | + | + | − |
| | | G1 | + | + | − |
| 30 | H268E | non-GE | − | − | nd* |
| | | G2 | − | − | − |
| | | G1 | − | − | − |
| 43 | S239D | non-GE | − | − | nd* |
| | | G2 | − | − | − |
| | | G1 | − | − | − |
| 85 | F243H | non-GE | + | + | nd* |
| | | G2 | = | = | − |
| 88 | T260H | non-GE | = | + | nd* |
| | | G2 | − | = | + |
| 98 | S239E | non-GE | − | − | nd* |
| | | G2 | − | − | − |

*= off-rates too fast for determination;
K_{D} was determined by steady state experiments.

TABLE 11
Table 11 - Oligosaccharide pattern (rel. %) of antibody mutants compared to the
control IgG.

| | | | 43 | | 20 | | 85 | | 22 | | 88 | | 9 | | 30 | |
|---|---|---|---|---|---|---|---|---|---|---|---|---|---|---|---|---|
| | control | | S239D | | S239W | | F243H | | F243E | | T260H | | H268D | | H268E | |
| n^{o} mutant | non- | | non- | | non- | | non- | | non- | | non- | | non- | | non- | |
| glycoform | GE | G2 | GE | G2 | GE | G2 | GE | G2 | GE | G2 | GE | G2 | GE | G2 | GE | G2 |
| complex | 100 | 93.1 | 100 | 91 | 100 | 87 | 100 | 94 | 100 | 91 | 100 | 93 | 100 | 93 | 100 | 92 |
| non- | 0 | 60.5 | 0 | 63 | 0 | 56 | 0 | 55 | 0 | 52 | 0 | 63 | 0 | 57 | 0 | 62 |
| fucosylated | | | | | | | | | | | | | | | | |
| bisected | 0 | 72.7 | 0 | 75 | 0 | 69 | 0 | 84 | 0 | 73 | 0 | 76 | 0 | 78 | 0 | 77 |

### Discussion

The selected IgG mutants were divided in three groups as described in Table 7.

Group 1—S239W, F243E, F243H

These antibody mutants have in their glycoengineered form very similar K_{D} values for their interaction with shFcγRIIIa-H6 as compared to the control glycoengineered antibody, but feature a decreased dissociation rate constant (4-fold decreased k_{off}). The affinity to shFcγRIIIa lacking glycosylation at position Q162 is decreased for these mutants in both glycoengineered and nonglycoengineered glycoforms as compared to the affinities displayed by the respective glycoforms for the control antibody. This indicates that the improved k_{off} results from the carbohydrate moiety and not from the amino acid mutation.

Group 2—H268D, H268E, S239D, S239E

These antibody mutants have a decreased K_{D} in both glycoengineered and nonglycoengineered glycoforms for shFcγRIIIa-H6 compared to the control antibody in the respective glycoforms. For the glycoengineered form, this is the result of a decreased dissociation rate constant (4- to 2-fold decreased k_{off}). On the contrary to the mutants of group 1, these antibodies also have, in both glycoengineered and nonglycoengineered glycoforms, increased affinities for shFcγRIIIa lacking glycosylation at position Q162, as compared to affinities displayed by the respective glycoforms of the control antibody, indicating the influence of the amino acid mutation in the improved affinity.

Group 3—T260H

The glycoengineered form of this mutant has a decreased K_{D} for sFcγRIIIa as compared to the glycoengineered control antibody, which is the result of an almost 3-fold increased kₒₙ for the glycoengineered mutant. The nonglycoengineered glycoform of this mutant has a similar affinity for sFcγRIIIa as compared to the nonglycoengineered control antibody. Binding to the shFcγRIIIa lacking glycosylation at position Q162 is slightly decreased for the nonglycoengineered glycoform of this mutant as compared to the nonglycoengineered control antibody, while binding for the glycoengineered mutant is similar to that of the glycoengineered control antibody.

The carbohydrate profiles of most selected mutants were analysed and indicate very similar oligosaccharide patterns compared to the control antibody.

### CONCLUSION

IgG mutants were identified that show an increased binding to hFcγRIIIa when non-fucosylated compared to the unmodified (fucosylated) antibody. Furthermore, some identified IgG mutants can be identified that have preferably an increased affinity to FcγRIIIa but not for FcγRIIIa-Q162 (which lacks glycosylation at position 162). Moreover, the described method allows the selection of IgG mutants with distinct characteristics, such as decreased k_{off} or increased kₒₙ.

1. A glycoengineered antigen binding molecule comprising a Fc region, wherein said Fc region has an altered oligosaccharide structure as a result of said glycoengineering and has at least one amino acid modification, and wherein said antigen binding molecule exhibits increased binding to, or increased specificity for, a human FcγRIII receptor compared to the antigen binding molecule that lacks said modification.

2. A glycoengineered antigen binding molecule according to claim 1, wherein said antigen binding molecule does not exhibit increased binding to a human FcγRII receptor.

3-4. (canceled)

5. A glycoengineered antigen binding molecule according to claim 1, wherein said FcγRIII receptor is glycosylated.

6. (canceled)

7. A glycoengineered antigen binding molecule according to claim 1, wherein said FcγRIII receptor is FcγRIIIa.

8. A glycoengineered antigen binding molecule according to claim 1, wherein said FcγRIII receptor is FcγRIIIb.

9. A glycoengineered antigen binding molecule according to claim 7, wherein said FcγRIIIa receptor has a valine residue at position 158.

10. A glycoengineered antigen binding molecule according to claim 7, wherein said FcγRIIIa receptor has a phenylalanine residue at position 158.

11. A glycoengineered antigen binding molecule according to claim 5, wherein said modification does not substantially increase binding to a nonglycosylated FcγRIII receptor compared to the antigen binding molecule lacking said modification.

12. A glycoengineered antigen binding molecule according to claim 1, wherein said Fc region comprises a substitution at one or more of amino acids 239, 241, 243, 260, 262, 263, 264, 265, 268, 290, 292, 293, 294, 295, 296, 297, 298, 299, 300, 301, 302, or 303.

13. (canceled)

14. A glycoengineered antigen binding molecule according to claim 1, wherein said Fc region comprises a substitution at one or more of amino acids 239, 243, 260, or 268.

15-16. (canceled)

17. A glycoengineered antigen binding molecule according to claim 14, wherein said substitution at one or more amino acids is selected from the group consisting of: Ser239Asp, Ser239Glu, Ser239Trp, Phe243His, Phe243Glu, Thr260His, His268Asp, or His268Glu.

18. A glycoengineered antigen binding molecule according to claim 17, wherein said substitution at more than one amino acid is selected from the substitutions listed in Table 5.

19. A glycoengineered antigen binding molecule according to claim 12, wherein said substitution is selected from a substitution listed in Table 2.

20. (canceled)

21. A glycoengineered antigen binding molecule according to claim 1, wherein said Fc region is a human IgG Fc region.

22. A glycoengineered antigen binding molecule according to claim 1, wherein said antigen binding molecule is an antibody or an antibody fragment comprising an Fc region.

23-24. (canceled)

25. A glycoengineered antigen binding molecule according to claim 1, wherein said antigen binding molecule exhibits increased effector function.

26. A glycoengineered antigen binding molecule according to claim 25, wherein said increased effector function is increased antibody-dependent cellular cytotoxicity or increased complement dependent cytotoxicity.

27. A glycoengineered antigen binding molecule according to claim 1, wherein said altered oligosaccharide structure comprises a decreased number of fucose residues as compared to the nonglycoengineered antigen binding molecule.

28-36. (canceled)

37. A glycoengineered antigen binding molecule according to claim 1, wherein said altered oligosaccharide structure comprises an increase in the ratio of GlcNAc residues to fucose residues as compared to the nonglycoengineered antigen binding molecule.

38. A glycoengineered antigen binding molecule according to claim 1, wherein said antigen binding molecule selectively binds an antigen selected from the group consisting of: the human CD20 antigen, the human EGFR antigen, the human MCSP antigen, the human MUC-1 antigen, the human CEA antigen, the human HER2 antigen, and the human TAG-72 antigen.

39-76. (canceled)

77. A polynucleotide encoding a polypeptide comprising an antibody Fc region or a fragment of an antibody Fc region, wherein said Fc region or fragment thereof has at least one amino acid modification, and wherein said polypeptide exhibits increased binding to a glycosylated human FcγRIII receptor compared to the same polypeptide that lacks said modification.

78. A polynucleotide according to claim 77, wherein said polypeptide is an antibody heavy chain.

79. A polynucleotide according to claim 77, wherein said polypeptide is a fusion protein.

80. A polypeptide encoded by the polynucleotide according to claim 77.

81. A polypeptide according to claim 80, wherein said polypeptide is an antibody heavy chain.

82. A polypeptide according to claim 80, wherein said polypeptide is a fusion protein.

83. An antigen binding molecule comprising a polypeptide according to claim 80.

84. A vector comprising the polynucleotide of claim 77.

85. A glycoengineered host cell comprising the vector of claim 84.

86. A method for producing a glycoengineered antigen binding molecule comprising a Fc region, wherein said Fc region has an altered oligosaccharide structure as a result of said glycoengineering and has at least one amino acid modification, and wherein said antigen binding molecule exhibits increased binding to, or increased specificity for, a human FcγRIII receptor compared to the antigen binding molecule that lacks said modification, said method comprising:
(a) culturing the glycoengineered host cell of claim 85 under conditions permitting the expression of said polynucleotide; and
(b) recovering said glycoengineered antigen binding molecule from the culture medium.

87-101. (canceled)

102. A pharmaceutical composition comprising the antigen binding molecule of claim 1 and a pharmaceutically acceptable carrier.

103. A method for the treatment or prophylaxis of cancer comprising administering a therapeutically effective amount of the pharmaceutical composition of claim 102 to a patient in need thereof.

104. The method according to claim 103, wherein said cancer is selected from the group consisting of breast cancer, bladder cancer, head and neck cancer, skin cancer, pancreatic cancer, lung cancer, ovarian cancer, colon cancer, prostate cancer, kidney cancer, and brain cancer.

105. A method for the treatment or prophylaxis of a precancerous condition or lesion comprising administering a therapeutically effective amount of the pharmaceutical composition of claim 102 to a patient in need thereof.

106. The method according to claim 105, wherein said precancerous condition or lesion is selected from the group consisting of oral leukoplakia, actinic keratosis (solar keratosis), precancerous polyps of the colon or rectum, gastric epithelial dysplasia, adenomatous dysplasia, hereditary nonpolyposis colon cancer syndrome (HNPCC), Barrett's esophagus, bladder dysplasia, and precancerous cervical conditions.

107. An antigen binding molecule according to claim 1 for use in the treatment or prophylaxis of cancer.

108. The antigen binding molecule according to claim 107, wherein said cancer is selected from the group consisting of breast cancer, bladder cancer, head and neck cancer, skin cancer, pancreatic cancer, lung cancer, ovarian cancer, colon cancer, prostate cancer, kidney cancer, and brain cancer.

109. An antigen binding molecule according to claim 1 for use in the treatment or prophylaxis of a precancerous condition or lesion.

110. The antigen binding molecule according to claim 109, wherein said precancerous condition or lesion is selected from the group consisting of oral leukoplakia, actinic keratosis (solar keratosis), precancerous polyps of the colon or rectum, gastric epithelial dysplasia, adenomatous dysplasia, hereditary nonpolyposis colon cancer syndrome (HNPCC), Barrett's esophagus, bladder dysplasia, and precancerous cervical conditions.

111. (canceled)
